(19) United States
(12) Patent Application Publication
(10) Pub. No.: US 2024/0084344 A1
(43) Pub. Date: Mar. 14, 2024

(54) PROCESSES FOR THE PRODUCTION OF TRYPTAMINES

(71) Applicant: Compass Pathfinder Limited, Altrincham (GB)

(72) Inventor: Ryan PROTZKO, Berkeley, CA (US)

(73) Assignee: Compass Pathfinder Limited, Altrincham (GB)

(21) Appl. No.: 18/301,790

(22) Filed: Apr. 17, 2023

Related U.S. Application Data
(63) Continuation of application No. 17/012,737, filed on Sep. 4, 2020, now Pat. No. 11,661,617.
Application No. 17/012,737 is a continuation of application No. PCT/US2019/021489, filed on Mar. 8, 2019.
(60) Provisional application No. 62/640,443, filed on Mar. 8, 2018.

(51) Int. Cl. C12P 17/10; A61K 31/4045; C07D 209/16; C12N 9/02; C12N 9/12; C12N 9/88

(52) U.S. Cl. CPC C12P 17/10; A61K 31/4045; C07D 209/16; C12N 9/0042; C12N 9/0071; C12N 9/0083; C12N 9/1205; C12N 9/88; C12Y 106/02004; C12Y 114/16004; C12Y 114/99; C12Y 207/01; C12Y 401/01

(57) ABSTRACT

Disclosed herein are prokaryotic and eukaryotic microbes, including E. coli and S. cerevisiae, genetically altered to biosynthesize tryptamine and tryptamine derivatives. The microbes of the disclosure may be engineered to contain plasmids and stable gene integrations containing sufficient genetic information for conversion of an anthranilate or an indole to a tryptamine. The fermentative production of substituted tryptamines in a whole-cell biocatalyst may be useful for cost effective production of these compounds for therapeutic use.

### CROSS-REFERENCE

This application is a continuation application of International Application No. PCT/US2019/021489, filed Mar. 8, 2019 which claims the benefit of U.S. Provisional Application No. 62/640,443, filed Mar. 8, 2018, which application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Sep. 2, 2020, is named 54033-701 301 SL.txt and is 201,159 bytes in size.

### BACKGROUND

Tryptamine is a monoamine alkaloid. It contains an indole ring structure, and is structurally similar to the amino acid tryptophan, from which the name derives. Tryptamine is found in trace amounts in the brains of mammals and is hypothesized to play a role as a neuromodulator or a neurotransmitter. Tryptamine is the common functional group in a set of compounds, termed collectively, substituted tryptamines. This set includes many biologically active compounds, including neurotransmitters and psychotropic drugs.

### SUMMARY

In one aspect, a microbial cell that produces a tryptamine is provided, the microbial cell containing therein one or more heterologous nucleic acid sequences encoding one or more enzymes involved in a biosynthesis pathway that converts an anthranilate to a tryptamine.

In another aspect, a microbial cell that produces a tryptamine is provided, the microbial cell containing therein one or more heterologous nucleic acid sequences encoding one or more enzymes involved in a biosynthesis pathway that converts an indole to a tryptamine.

In another aspect, a microbial cell that produces a tryptamine is provided, the microbial cell containing therein one or more heterologous nucleic acid sequences encoding one or more enzymes involved in a biosynthesis pathway that converts tryptophan to a tryptamine.

In some cases, the anthranilate is a substituted anthranilate. In some cases, the anthranilate is:

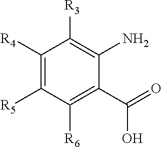

where:
each R is independently a hydrogen, a halogen, —OH, C₁-C₅ alkyl, C₁-C₅ alkoxy, NO₂, NH, COOH, CN, sulfur, SO₃, SO₄, or PO₄.

In some cases, the indole is a substituted indole. In some cases, the indole is:

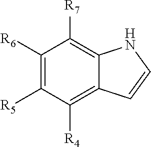

where:
each R is independently a halogen, —OH, C₁-C₅ alkyl, C₁-C₅ alkoxy, NO₂, NH, COOH, CN, sulfur, SO₃, SO₄, or PO₄.

In some cases, the tryptamine is a substituted tryptamine. In some cases, the tryptamine is:

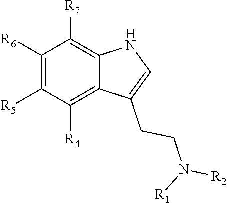

where:
each R is independently a hydrogen, a halogen, —OH, C₁-C₅ alkyl, C₁-C₅ alkoxy, NO₂, NH, COOH, CN, sulfur, SO₃, SO₄, or PO₄.

In some cases, the one or more enzymes comprise one or more of: trpD, trpB, trpC, and trpA. In some cases, the one or more heterologous nucleic acid sequences comprises a multicistronic operon encoding at least two of trpD, trpB, trpC, and trpA. In some cases, the multicistronic operon has a nucleic acid sequence having at least 80% sequence identity to any one of SEQ ID NOs: 1-4. In some cases, the trpD comprises an amino acid sequence having at least 80% sequence identity to any one of SEQ ID NOs: 5-7. In some cases, the trpC comprises an amino acid sequence having at least 80% sequence identity to any one of SEQ ID NOs: 8 and 9. In some cases, the trpB comprises an amino acid sequence having at least 80% sequence identity to any one of SEQ ID NOs: 10 and 11. In some cases, the trpA comprises an amino acid sequence having at least 80% sequence identity to any one of SEQ ID NOs: 12 and 13. In some cases, the one or more enzymes comprise a decarboxylase. In some cases, the decarboxylase is a tryptophan decarboxylase. In some cases, the tryptophan decarboxylase comprises an amino acid sequence having at least 50%, at least 60%, at least 70%, at least 80%, at least 90%, or at least 95% sequence identity to any one of SEQ ID NOs: 14-20. In some cases, the one or more enzymes comprise a transferase. In some cases, the transferase is selected from the group consisting of: tryptamine N-methyltransferase, tryptamine benzoyl transferase, serotonin N-acetyltransferase, dopamine N-acetyltransferase, arylalkylamine N-acetyltransferase, and tryptamine hydroxycinnamoyltransferase. In some cases, the transferase comprises an amino acid sequence having at least 50% sequence identity to any one of SEQ ID NOs: 21-31 or 46. In some cases, the one or more enzymes comprise tryptamine 4-hydroxylase. In some cases, the tryptamine 4-hydroxylase comprises an amino acid sequence having at least 50%, at least 60%, at least 70%, at least 80%, at least 90%, or at least 95% sequence identity to any one of SEQ ID NOs: 32-35. In some cases, the one or more enzymes comprises a P450 reductase. In some cases, the P450 reductase comprises an amino acid sequence having at least 50%, at least 60%, at least 70%, at least 80, at least 90%, or at least 95% sequence identity to any one of SEQ ID NOs: 36-40. In some cases, the one or more enzymes comprises a kinase. In some cases, the kinase comprises an amino acid sequence having at least 50%, at least 60%, at least 70%, at least 80%, at least 90%, or at least 95% sequence identity to any one of SEQ ID NOs: 41-44. In some cases, the anthranilate is biosynthetically produced by the microbial cell. In some cases, the anthranilate is fed to the engineered microbial cell. In some cases, the anthranilate is 5-bromoanthranilate, 6-hydroxyanthranilate, 5-hydroxyanthranilate, 6-chloroanthranilate, or 5-chloroanthranilate. In some cases, the indole is biosynthetically produced by the microbial cell. In some cases, the indole is fed to the engineered microbial cell. In some cases, the indole is selected from the group consisting of: 5-hydroxyindole, 4-hydroxyindole, 7-hydroxyindole, and 4-chloroindole, 5-bromoindole, or 4-fluoroindole. In some cases, the microbial cell secretes the tryptamine in culture broth. In some cases, the tryptamine is selected from any tryptamine described in FIG. 4, FIG. 6, or FIG. 8. In some cases, the tryptamine is selected from the group consisting of: tryptamine, 5-hydroxytryptamine, 5-hydroxymethyltryptamine, 5-hydroxy-N,N-dimethyltryptamine, 5-phosphoryloxymethyltryptamine, 5-phosphoryloxy-N,N-dimethyltryptamine, 4-hydroxytryptamine, 4-hydroxy-N,N-dimethyltryptamine, 4-phosphoryloxytryptamine, 4-phosphoryloxy-N,N-tryptamine, 7-hydroxytryptamine, 7-phosphoryloxymethyltryptamine, 7-phosphoryloxy-N,N-dimethyltryptamine, 4-chloro-tryptamine, 4-chloro-N,N-dimethyltryptamine, 5-bromotryptamine, 5-bromo-methyltryptamine, 5-bromo-N-methyltryptamine, 5-bromo-N,N-dimethyltryptamine, N-acetyl-tryptamine, 4-hydroxy-N-acetyl-tryptamine. In some cases, the microbial cell is a eukaryotic cell. In some cases, the microbial cell is a yeast cell. In some cases, the yeast cell is of the species Saccharomyces cerevisiae. In some cases, the yeast cell does not express one or more of aromatic aminotransferase I (aro8) and phenylpyruvate decarboxylase (aro10). In some cases, the yeast cell overexpresses one or more of phosphoribosylanthranilate isomerase (TRP1), anthranilate synthase (TRP2), indole-3-glycerolphosphate synthase (TRP3), anthranilate phosphoribosyl transferase (TRP4), and tryptophan synthase (TRPS). In some cases, the yeast cell overexpresses a mutant of one or more of phosphoribosylanthranilate isomerase (TRP1), anthranilate synthase (TRP2), indole-3-glycerolphosphate synthase (TRP3), anthranilate phosphoribosyl transferase (TRP4), and tryptophan synthase (TRPS). In some cases, the yeast cell has two or more copies of the one or more heterologous nucleic acid sequences and they act synergistically. In some cases, the microbial cell is a prokaryote. In some cases, the microbial cell is a bacterial cell. In some cases, the bacterial cell is of the species Escherichia coli or Corynebacterium glutamicum. In some cases, the bacterial cell does not express one or more of tryptophanase (tna), tryptophan repressor element (trpR), or anthranilate synthase (trpE) genes. In some cases, at least one copy of the one or more heterologous nucleic acid sequences is stably integrated into the genome of the microbial cell. In some cases, two or more copies of the one or more heterologous nucleic acid sequences are stably integrated into the genome of the microbial cell. In some cases, the two or more copies of the one or more heterologous nucleic acid sequences are from a same sequence. In some cases, the two or more copies of the one or more heterologous nucleic acid sequences are from a distinct sequence.

In another aspect, a method for synthesizing a tryptamine is provided, the method comprising: culturing a microbial cell according to any of the preceding in a presence of anthranilate, thereby synthesizing the tryptamine. In some cases, the method further comprises feeding the anthranilate to the microbial cell. In some cases, the anthranilate is produced biosynthetically by the microbial cell. In some cases, the anthranilate is a substituted anthranilate.

In another aspect, a method for synthesizing a tryptamine is provided, the method comprising: culturing a microbial cell according to any of the preceding in a presence of indole, thereby synthesizing the tryptamine. In some cases, the method further comprises feeding the indole to the microbial cell. In some cases, the indole is produced biosynthetically by the microbial cell. In some cases, the indole is a substituted indole.

In another aspect, a method for synthesizing a tryptamine is provided, the method comprising: culturing a microbial cell of any of the preceding in a presence of tryptophan, thereby synthesizing the tryptamine. In some cases, the method further comprises feeding the tryptophan to the microbial cell. In some cases, the tryptophan is produced biosynthetically by the microbial cell.

In some cases, any method of the preceding further comprises purifying the tryptamine from the culture.

In another aspect, a microbial cell is provided containing therein one or more heterologous nucleic acid sequences encoding one or more enzymes involved in a biosynthesis pathway to convert a tryptamine to a tryptamine derivative. In some cases, the one or more enzymes comprise a tryptamine 4-hydroxylase. In some cases, tryptamine 4-hydroxylase comprises an amino acid sequence having at least 50%, at least 60%, at least 70%, at least 80%, at least 90%, or at least 95% sequence identity to any one of SEQ ID NOs:32-35. In some cases, the one or more enzymes comprise a tryptamine 5-hydroxylase. In some cases, the tryptamine 5-hydroxylase comprises an amino acid sequence having at least 50%, at least 60%, at least 70%, at least 80%, at least 90%, or at least 95% sequence identity to SEQ ID NO:47. In some cases, the one or more enzymes comprise a 4-hydroxytryptamine kinase. In some cases, the 4-hydroxytryptamine kinase comprises has an amino acid sequence having at least 50%, at least 60%, at least 70%, at least 80%, at least 90%, or at least 95% sequence identity according to any one of SEQ ID NOs:41-44. In some cases, the tryptamine is a substituted tryptamine. In some cases, the tryptamine is selected from the group consisting of: 5-methoxy-N,N-dimethyl-tryptamine, N,N-diisopropyl-tryptamine, N-methyl-N-isopropyltryptamine, N,N-dimethyltryptamine, N,N-tetramethylenetryptamine, N,N-dipropyltryptamine, 4-hydroxy-N,N-dimethyltryptamine, tryptamine, 4-hydroxytryptamine, 5-hydroxytryptamine, ibogamine, 4-hydroxyibogamine, and 5-hydroxyibogamine. In some cases, the tryptamine derivative is any tryptamine derivative described in FIG. 16. In some cases, the tryptamine derivative is selected from the group consisting of: 5-hydroxy-N,N-diisopropyl-tryptamine, 5-hydroxy-N-methyl-N-isopropyltryptamine, 5-hydroxy-N,N-dimethyltryptamine, 5-hydroxy-N,N-tetramethylenetryptamine, 5-hydroxy-N,N-dipropyltryptamine, 4,5-methoxy-N,N-dimethyl-tryptamine, 4-hydroxy-N,N-diisopropyl-tryptamine, 4-hydroxy-N-methyl-N-isopropyltryptamine, 4-hydroxy-N,N-dimethyltryptamine, 4-hydroxy-N,N-tetramethylenetryptamine, 4-hydroxy-N,N-dipropyltryptamine, 4-phosphoryloxy-N,N-dipropyltryptamine, 4-hydroxytryptamine, 5-hydroxytryptamine, 4-methoxytryptamine, 5-methoxytryptamine, 4-phosphoryloxytryptamine, 5-phosphoryloxytryptamine, 4-hydroxyibogamine, 5-hydroxyibogamine, 4-phosphoryloxyibogamine, and 5-phosphoryloxyibogamine.

In another aspect, a method of synthesizing a tryptamine derivate from a tryptamine is provided, the method comprising: culturing a microbial cell according to any of the preceding in a presence of a tryptamine, thereby synthesizing the tryptamine derivative. In some cases, the method further comprises purifying the tryptamine derivative from the culture.

In yet another aspect, a vector is provided comprising one or more heterologous nucleic acid sequences encoding one or more enzymes comprising an amino acid sequence having at least 50%, at least 60%, at least 70%, at least 80%, at least 90%, or at least 95% sequence identity to any one of SEQ ID NOs: 5-49.

In yet another aspect, a microbial cell is provided containing therein one or more heterologous nucleic acid sequences encoding an enzyme from a tryptamine synthesis pathway or a functional fragment thereof comprising an amino acid sequence having at least 50%, at least 60%, at least 70%, at least 80%, at least 90%, or at least 95% sequence identity to any one of SEQ ID NOs: 5-49.

In another aspect, a method is provided for screening for the levels of 4-hydroxytryptamine within any microbial cell of the preceding, the method comprising: detecting a color or a fluorescence product of the 4-hydroxytryptamine within the microbial cell. In some cases, the 4-hydroxytryptamine is oxidized within the microbial cell, thereby producing an oxidized 4-hydroxytryptamine. In some cases, the oxidized 4-hydroxytryptamine is directly proportional to a level of 4-hydroxytryptamine synthesized within the microbial cell. In some cases, an oxidation of the oxidized 4-hydroxytryptamine is catalyzed by iron sulphate. In some cases, an oxidation of the oxidized 4-hydroxytryptamine is catalyzed by an enzyme expressed by the microbial cell. In some cases, the enzyme comprises an amino acid sequence having at least 50%, at least 60%, at least 70%, at least 80%, at least 90%, or at least 95% sequence identity to SEQ ID NO: 45.

In another aspect, a method of converting an anthranilate to a tryptamine is provided, the method comprising incubating the anthranilate in a presence of one or more enzymes involved in a biosynthesis pathway that converts an anthranilate to a tryptamine.

In yet another aspect, a method of converting an indole to a tryptamine is provided, the method comprising incubating the indole in a presence of one or more enzymes involved in a biosynthesis pathway that converts an indole to a tryptamine.

In yet another aspect, a method of converting tryptophan to a tryptamine is provided, the method comprising incubating the tryptophan in a presence of one or more enzymes involved in a biosynthesis pathway that converts tryptophan to a tryptamine.

In yet another aspect, a method of converting a tryptamine to a derivatized tryptamine is provided, the method comprising incubating the tryptamine in a presence of one or more enzymes involved in a biosynthetic pathway that converts tryptamine to a derivatized tryptamine.

In some cases, a method of the preceding is performed in the absence of a biological cell. In some cases, a method of the preceding is performed under in vitro conditions. In some cases, a method of the preceding is performed under cell-free conditions. In some cases, a method of the preceding is performed in a cell lysate.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Some novel features of the invention are set forth in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a non-limiting example of a plasmid map suitable for overexpression of the bacterial tryptophan operon in bacteria in accordance with embodiments of the disclosure. Plasmid is medium copy (p15a) and contains ampicillin resistance. TrpD, trpC, trpB and trpA are expressed in a multicistronic operon (e.g., SEQ ID NO: 1).

FIG. 2 depicts a non-limiting example of a plasmid map suitable for overexpression of psilocybin synthase (e.g., SEQ ID NO: 21), tryptophan decarboxylase (e.g., SEQ ID NO: 14) and 4-hydroxytryptamine kinase (e.g., SEQ ID NO: 41) in bacteria in accordance with embodiments of the disclosure.

FIG. 3 depicts a non-limiting example of a plasmid map suitable for the production of N-methyl derivatives in bacteria by overexpression of tryptophan decarboxylase (e.g., SEQ ID NO: 19) and ethanolamine methyltransferase (e.g., SEQ ID NO: 25) in accordance with embodiments of the disclosure.

FIG. 4 depicts non-limiting examples of substituted tryptamines produced by engineered bacterial cells fed with substituted anthranilate or indole compounds in accordance with embodiments of the disclosure.

FIG. 5 depicts a non-limiting example of a plasmid map suitable for the production of N-methyl tryptamine derivatives in yeast by overexpression of tryptophan decarboxylase (e.g., SEQ ID NO: 19), 4-hydroxytryptamine kinase (e.g., SEQ ID NO: 41), psilocybin synthase (e.g., SEQ ID NO: 21) and tryptamine 4-hydroxylase (e.g., SEQ ID NO: 33) in accordance with embodiments of the disclosure.

FIG. 6 depicts a non-limiting example of a biosynthetic pathway converting anthranilic acid present in yeast metabolism to tryptamines by enzymes encoded in pRJP1608 in accordance with embodiments of the disclosure.

FIG. 7 depicts a non-limiting example of a plasmid map for the production of N-acetyl tryptamine derivatives in yeast by overexpression of tryptophan decarboxylase (e.g., SEQ ID NO: 19), tryptamine 4-hydroxylase (e.g., SEQ ID NO: 33) and N-acetyltransferase (e.g., SEQ ID NO: 28) in accordance with embodiments of the disclosure.

FIG. 8 depicts a non-limiting example of a biosynthetic pathway converting anthranilic acid present in yeast metabolism to tryptamines by enzymes encoded in pRJP1618 is accordance with embodiments of the disclosure.

FIG. 9 depicts a non-limiting example of tandem mass spectrometry (MS/MS) of 4-hydroxy-N,N-dimethyl-tryptamine derived from engineered yeast cells in accordance with embodiments of the disclosure.

FIG. 10 depicts a non-limiting example of tandem mass spectrometry (MS/MS) of psilocybin derived from engineered yeast cells in accordance with embodiments of the disclosure.

FIG. 11 depicts a non-limiting example of tandem mass spectrometry (MS/MS) of N-acetyltryptamine derived from engineered yeast cells in accordance with embodiments of the disclosure.

FIG. 12 depicts a non-limiting example of tandem mass spectrometry (MS/MS) of 4-hydroxy-N-acetyltryptamine derived from engineered yeast cells in accordance with embodiments of the disclosure.

FIG. 13 depicts a non-limiting example of a plasmid map for the overexpression tryptamine 4-hydroxylase (e.g., SEQ ID NO: 33) in yeast in accordance with embodiments of the disclosure.

FIG. 14 depicts a non-limiting example of a plasmid map for the overexpression of tryptamine 5-hydroxylase (e.g., SEQ ID NO: 47) in yeast in accordance with embodiments of the disclosure.

FIG. 15 depicts a non-limiting example of a plasmid map for the overexpression of 4-hydroxytryptamine kinase (e.g., SEQ ID NO: 41) in yeast in accordance with embodiments of the disclosure.

FIG. 16 depicts a non-limiting example of the production of substituted tryptamines from fed tryptamines using engineered yeast in accordance with embodiments of the disclosure.

FIG. 17 depicts a non-limiting example of a colorimetric screening assay as an indicator of hydroxylase activity in yeast in accordance with embodiments of the disclosure.

FIG. 18A depicts a non-limiting example of a biosynthetic pathway converting tryptamine to tryptamine derivatives.

FIG. 18B depicts a non-limiting example of a biosynthetic pathway converting ibogamine to derivatives of ibogamine.

### DETAILED DESCRIPTION

The present disclosure relates to microorganisms containing heterologous DNA useful in the production of tryptamines with 4-, 5-, 6-, or 7-indole substitutions, and/or R1 or R2 amine substitutions. Furthermore, this disclosure relates to processes for optimizing production, executing production, and recovering such substituted tryptamines. The disclosure provided herein provides processes for the production of various compounds, such as tryptamines. The disclosure further provides prokaryotic and eukaryotic microbes, including bacteria (e.g., Escherichia coli) and yeast (e.g., Saccharomyces cerevisiae), that may be genetically altered to contain heterologous sequences that encode biological molecules that can provide a biosynthetic pathway for the synthesis of tryptamine and/or substituted tryptamines in vivo. In some aspects, the disclosure provides microbes that may be engineered to contain plasmids and stable gene integrations containing sufficient genetic information for conversion of anthranilate or substituted anthranilates, and/or indole or substituted indoles, to a respective tryptamine or substituted tryptamine. The fermentative production of substituted tryptamines in a whole-cell biocatalyst may be useful for cost effective production of these compounds for therapeutic use.

Tryptamines are naturally occurring monoamine alkaloids derived from tryptophan, from which the name is derived. Analogs within the tryptamine family contain substitutions at the indole ring structure and the amine group. This family of compounds contains psychotropically active members, including N,-N-dimethyltryptophan (DMT), 5-methoxy-N,N-dimethyltryptamine (5-MeO-DMT), 4-hydroxy dimethyl-tryptophan (psilocin) and its 4-O-phosphate ester, psilocybin (Hofmann et al. 1959). Psilocin may act as a partial agonist on 5HT1a, 5HT2a, and 5HT2c receptors (Hasler et al. 2004). Several basidiomycete fungi of the genus Psilocybe and other genera produce substituted tryptamines biosynthetically, including psilocybin, psilocin, norpsilocin, baeocystin, norbaeocystin and aeruginascin (Lenz, Wick, and Hoffmeister 2017). The compound N,N-dimethyltryptamine is ubiquitous in nature and is produced by many plants and animals (Carbonaro and Gatch 2016). Substituted tryptamines can also be synthetically derived, including the tryptans (e.g., Zolmitriptan and Sumatriptan), which are chemically synthesized and used as medications used to treat migraines and cluster headaches (Derry, Derry, and Moore 2014).

Tryptamines, such as psilocin, can cause profound changes in perception and mood in human subjects. Administration of high-dose psilocybin has been found to reliably induce mystical experiences leading to significant and enduring improvements in quality of life (Griffiths et al. 2006). Psilocybin administration has been concluded to be safe and well tolerated on 9 patients with severe, refractory obsessive-compulsive disorder and may be associated with “robust acute reductions” in core symptoms (Moreno et al. 2006).

Due to their complex structure, tryptamines and their respective substituted analogs are difficult to obtain commercially at economically feasible prices, if at all in large scale. Several organic chemistry methods exist for production of substituted tryptamines, including psilocybin. Dr. Albert Hoffmann originally published on the organics synthesis of psilocybin in 1958 (Hofmann et al. 1958). However, a dangerous reagent was used to phosphorylate the phosphate at the −4 position of the indole ring and later improvements were made for the synthesis (Hofmann, A. & Troxler, F. 1963. Esters of Indoles (U.S. Pat. No. 3,075,992), Basel, Switzerland: Sandoz Ltd.). This production method was adopted by Dr. David E Nichols for early clinical trials, but at a high cost for production (Nichols 2014).

Extraction of tryptamines from basidiomycete fungal tissue naturally producing the compounds is not suitable for large scale up production. The reported concentrations of psilocybin in mushrooms Psilocybe cubensis are less than 1% of the dry cell weight (J. Gartz 1994), causing a challenge for extraction and purification. Furthermore, the cultivation of such fungal tissue requires month-long time scales and would cause supply challenges (Jochen Gartz, Allen, and Merlin 1994). Furthermore, use of natural tissue precludes the ability to produce novel and unnatural tryptamine compounds with therapeutic properties.

The instant disclosure provides methods and materials to produce substituted tryptamines in high yield from inexpensive media components. The methods of the disclosure provide for production of tryptamine derivatives not naturally found in nature or tryptamine derivatives that are not accessible by synthetic chemistry. In some instances, the disclosed tryptamine derivatives may have favorable pharmacological effects (e.g., half-life, indications, etc). Additional advantages of the methods described herein include the use of a single biocatalyst for production of several substituted tryptamine analogues and a whole cell catalyst that is robust in fermentation and can regenerate itself for ease of use during production runs.

Accordingly, the objective of the present invention is to provide novel processes for the biosynthetic production of 4-, 5-, 6- or 7- indole substituted and/or R1 or R2 amine substituted tryptamines.

In some cases of the present disclosure, 4-, 5-, 6- or 7- indole substituted and/or R1 or R2 amine substituted tryptamines may be biosynthetically produced from corresponding substituted anthranilates and indoles by engineered microbial cells. Substituted anthranilates and indoles are widely available, vast in variety, and inexpensive compared to their respective substituted tryptamines.

In other aspects of the disclosure, a method of converting an anthranilate to a tryptamine is provided, the method comprising incubating the anthranilate in the presence of one or more enzymes involved in a biosynthesis pathway that converts an anthranilate to a tryptamine. In other aspects of the disclosure, a method of converting an indole to a tryptamine is provided, the method comprising incubating the indole in the presence of one or more enzymes involved in a biosynthesis pathway that converts an indole to a tryptamine. In other aspects of the disclosure, a method of converting tryptophan to a tryptamine is provided, the method comprising incubating the tryptophan in the presence of one or more enzymes involved in a biosynthesis pathway that converts tryptophan to a tryptamine. In other aspects of the disclosure, a method of converting a tryptamine to a derivatized tryptamine is provided, the method comprising incubating the tryptamine in the presence of one or more enzymes involved in a biosynthetic pathway that converts tryptamine to a derivatized tryptamine. In some cases, the methods may be performed within a biological cell (e.g., by an engineered microbial cell as described herein). In other cases, the methods may be performed in the absence of a biological cell. In some cases, the methods may be performed under in vitro conditions. In some cases, the methods may be performed under cell-free conditions. In some cases, the methods may be performed in a cell lysate.

### Synthesis of a Substituted Tryptamine from a Substituted Anthranilate in Engineered Microbial Cells

In an aspect of the disclosure, the processes described herein provide for the production of 4-, 5-, 6- or 7- indole substituted tryptamines with R1 or R2 substitutions at the amine. In some cases, anthranilate or an anthranilate substituted at 3- 4-, 5-, or 6- can be used to make 4-, 5-, 6- or 7- indole substituted tryptamines with R1 or R2 substitutions. In some cases, the process may be carried out in a whole-cell microbial fermentation. In some cases, an engineered microbial cell may be cultured in the presence of anthranilate or a substituted anthranilate (e.g., anthranilate or substituted anthranilate may be fed to or otherwise incubated with the microbial cell). In other cases, the anthranilate or substituted anthranilate may be produced biosynthetically by the microbial cell. For example, a microbial cell may produce anthranilate or a substituted anthranilate naturally (e.g., as part of central carbon metabolism). In other cases, the microbial cell may be engineered to produce anthranilate or a substituted anthranilate (e.g., by overexpressing enzymes for the production of substituted anthranilates).

Scheme 1 below depicts a non-limiting example of synthesis of a substituted tryptamine from anthranilate or a substituted anthranilate in an engineered microbial cell.

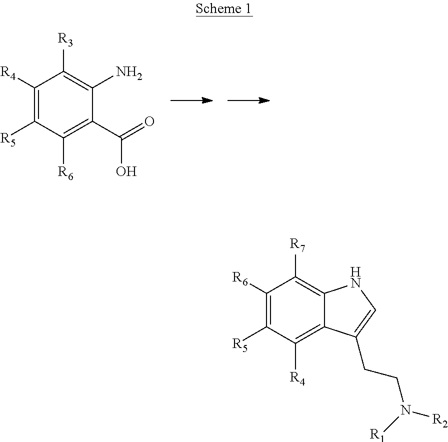

In some aspects, the disclosure provides a method for the production of substituted tryptamines by cultivating engineered microbes in the presence of anthranilate or a substituted anthranilate,

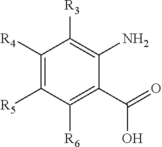

where —R is, but is not limited to, a halogen (—Br, —F, —Cl, —I, etc), —OH, C1-C5 alkyl, C1-C5 alkoxy, NO₂, NH, COOH, CN, sulfur, SO₃, SO₄, or PO₄. The resulting substituted tryptamine,

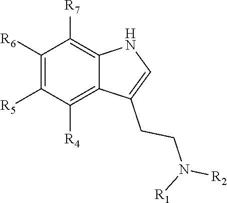

may be recovered from the culture broth. In some cases, the resulting tryptamine may be used in further downstream chemistry, taking advantage of chemical leaving groups or protecting groups incorporated into the tryptamine scaffold during the fermentative biosynthetic process.

In another aspect of the disclosure, indole or indole substituted at 4-, 5-, 6-, or 7- can be used to make 4-, 5-, 6-, or 7- indole substituted tryptamines with R1 or R2 substitutions. In some cases, the process may be carried out in a whole-cell microbial fermentation. In some cases, an engineered microbial cell may be cultured in the presence of indole or a substituted indole (e.g., indole or substituted indole may be fed to or otherwise incubated with the microbial cell). In other cases, the indole or substituted indole may be produced biosynthetically by the microbial cell. For example, a microbial cell may produce indole or a substituted indole naturally. In other cases, the microbial cell may be engineered to produce indole or a substituted indole (e.g., by overexpressing enzymes for the production of substituted indoles).

### Synthesis of a Substituted Tryptamine From Indole or a Substituted Indole in Engineered Microbial Cells

Scheme 2 depicts a non-limiting example of synthesis of a substituted tryptamine from indole or a substituted indole in an engineered microbial cell.

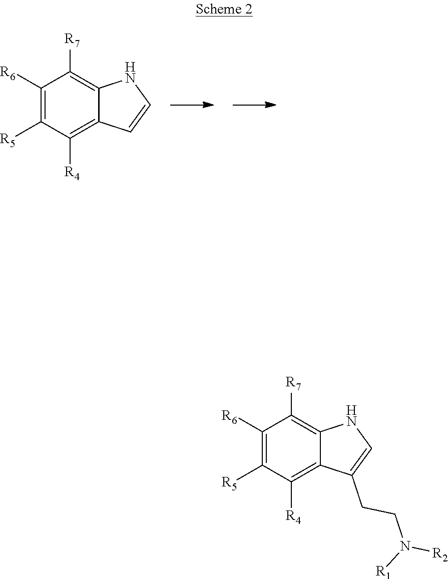

In some aspects, the disclosure provides a method for the production of substituted tryptamines by cultivating engineered microbes in the presence of indole or a substituted indole,

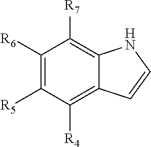

where —R is, but is not limited to, a halogen (—Br, —F, —Cl, —I, etc), —OH, C1-C5 alkyl, C1-C5 alkoxy, NO₂, NH, COOH, CN, sulfur, SO₃, SO₄, or PO₄. The resulting substituted tryptamine,

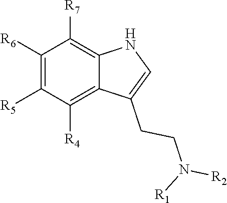

may be recovered from the culture broth. In some cases, the resulting tryptamine may be used in further downstream chemistry, taking advantage of chemical leaving groups or protecting groups incorporated into the tryptamine scaffold during the fermentative biosynthetic process.

### Synthesis of a Substituted Tryptamine From Anthranilate or a Substituted Anthranilate in Engineered Microbial Cells

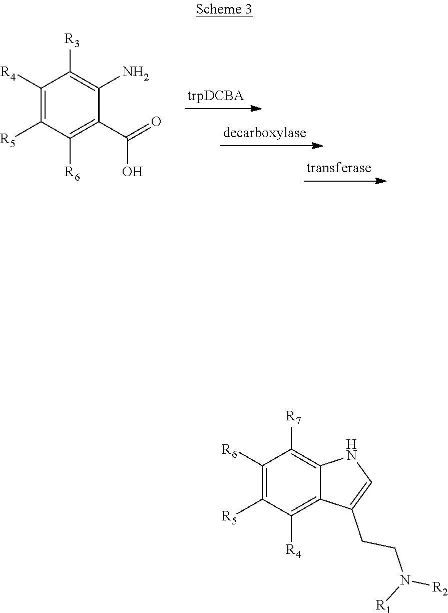

In some aspects, the processes described herein may involve the use of engineered microbes for the production of substituted tryptamines from anthranilate or substituted anthranilate. Scheme 3 depicts a non-limiting example of production of a substituted tryptamine from a substituted anthranilate in an engineered microbial cell. In some cases, the engineered microbial cell may be a bacterial cell. In some cases, the bacteria may be Escherichia coli or Corynebacterium glutamicum. In some cases, the bacteria may comprise modified host genetics, including knockout of tna (tryptophanase), trpR (tryptophan repressor element), and trpE (anthranilate synthase). In some cases, the engineered microbial cell may be a yeast cell. In some cases, the yeast cell may be of the species Saccharomyces cerevisiae. In some cases, the microbial cell may be further modified to express or overexpress one or more genes. In some cases, the microbial cell may be engineered to contain extra DNA copies by plasmid or genomic integration of an endogenous or heterologous trpDCBA operon. In some cases, the trpDCBA operon may comprise any one of SEQ ID NOs: 1-4. In some cases, the trpDCBA operon may comprise a nucleic acid sequence having at least 40%, at least 50%, at least 60%, at least 70%, at least 80%, at least 90%, or at least 95% sequence identity to any one of SEQ ID NOs: 1-4. In some cases, the engineered microbial cell may produce one or more enzymes having an amino acid sequence according to any one of SEQ ID NOs: 5-13, or an amino acid sequence having at least 40%, at least 50%, at least 60%, at least 70%, at least 80%, at least 90%, or at least 95% sequence identity to any one of SEQ ID NOs: 5-13. In some cases, the engineered microbial cell may produce one or more of trpD, trpB, trpC, or trpA, wherein the enzyme has been modified or mutated to exhibit higher levels of activity.

In some cases, the microbial cell may be further engineered to express or overexpress one or more additional genes. In some aspects, such microbial cell may further express a tryptamine decarboxylase (see Scheme 3, “decarboxylase”). In some cases, the tryptamine decarboxylase may be expressed by genomic integration of DNA or expression of a plasmid in the microbial cell. Tryptamine decarboxylases may be pyridoxal phosphate (PLP)-independent or may be PLP-dependent. In some cases, a tryptamine decarboxylase may comprise any one of the amino acid sequences according to SEQ ID NOs: 14-20 (see Table 2), or an amino acid sequence having at least 40%, at least 50%, at least 60%, at least 70%, at least 80%, at least 90%, or at least 95% sequence identity to an amino acid sequence of any one of SEQ ID NOs: 14-20. In some cases, an engineered microbial cell may express a tryptamine decarboxylase that has been modified or mutated to exhibit higher activity levels.

In some cases, the R1 and R2 amino positions of the tryptamine or substituted tryptamines derived from fermentation can be modified by a transferase to yield, by non-limiting example, N-methyl, N,N-dimethyl, N-acetyl, or N-hydroxycinnamoyl functional groups. Thus, in some cases, an engineered microbial cell may further express or overexpress a transferase (see Scheme 3). In some cases, a transferase may comprise any one of the amino acid sequences shown in SEQ ID NOs: 21-31 (see Table 2), or an amino acid sequence having at least 40%, at least 50%, at least 60%, at least 70%, at least 80%, at least 90%, or at least 95% sequence identity to an amino acid sequence of any one of SEQ ID NOs: 21-31. In some cases, an engineered microbial cell may express a transferase that has been modified or mutated to exhibit higher activity levels.

In some cases, an additional transferase can be expressed, such as, but not limited to, a phosphotransferase (kinase), acetyl transferase, glucosyl transferase, or sulfotransferase, to further modify hydroxyls on the indole ring of the tryptamine. For example, such as when engineered cells are cultivated in the presence of 6-hydroxyanthranilate or 4-hydroxy indole to yield 4-hydroxy-N,N-dimethyltryptamine, a kinase can be expressed yielding the phosphate ester of 4-hydroxy-N,N-dimethyltryptamine, psilocybin. Suitable kinases may include, but are not limited to, an amino acid sequence shown in any one of SEQ ID NOs: 41-44 (see Table 2), or an amino acid sequence having at least 40%, at least 50%, at least 60%, at least 70%, at least 80%, at least 90%, or at least 95% sequence identity to an amino acid sequence of any one of SEQ ID NOs: 41-44.

In some cases, the substituted anthranilate may be any one of 5-bromoanthranilate, 6-hydroxyanthranilate, 5-hydroxyanthranilate, 6-chloroanthranilate, and 5-chloroanthranilate. In some cases, the tryptamine may be any one of tryptamine, 5-hydroxytryptamine, 5-hydroxymethyltryptamine, 5-hydroxy-N,N-dimethyltryptamine, 5-phosphoryloxymethyltryptamine, 5-phosphoryloxy-N,N-dimethyltryptamine, 4-hydroxytryptamine, 4-hydroxy-N,N-dimethyltryptamine, 4-phosphoryloxytryptamine, 4-phosphoryloxy-N,N-tryptamine, 7-hydroxytryptamine, 7-phosphoryloxymethyltryptamine, 7-phosphoryloxy-N,N-dimethyltryptamine, 4-chloro-tryptamine, 4-chloro-N,N-dimethyltryptamine, 5-bromotryptamine, 5-bromo-methyltryptamine, 5-bromo-N-methyltryptamine, 5-bromo-N,N-dimethyltryptamine, N-acetyl-tryptamine, and 4-hydroxy-N-acetyl-tryptamine.

### Synthesis of a Substituted Tryptamine From Indole or a Substituted Indole in Engineered Microbial Cells

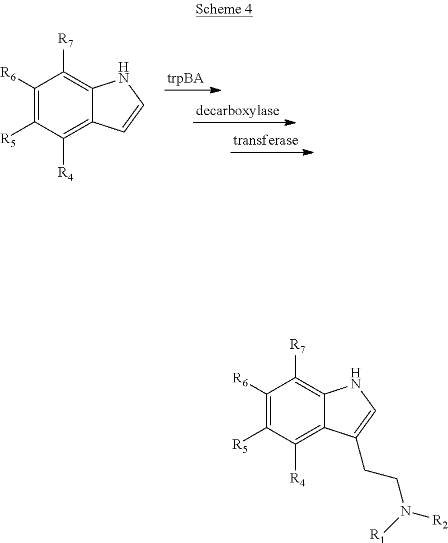

In some aspects, the processes described herein may involve the use of engineered microbes for the production of substituted tryptamines from indole or substituted indole. Scheme 4 depicts a non-limiting example of production of a substituted tryptamine from a substituted indole in an engineered microbial cell. In some cases, the engineered microbial may be a bacterial cell. In some cases, the bacterial cell may be of the species Escherichia coli or Corynebacterium glutamicum. In some cases, the bacterial cell may comprise modified host genetics, including knockout of tna (tryptophanase), trpR (tryptophan repressor element), and trpE (anthranilate synthase). In some cases, the microbial cell may be a yeast cell. In some cases, the yeast cell may be of the species Saccharomyces cerevisiae.

In some cases, the microbial cell may be further modified to express or overexpress one or more genes. In some cases, the microbial cell may be engineered to contain extra DNA copies by plasmid or genomic integration of endogenous or heterologous trpB and trpA (see, e.g., Scheme 4). In some cases, trpB and trpA may comprise amino acid sequences according to any one of SEQ ID NOs; 5-13 (see Table 2), or an amino acid sequence having at least 40%, at least 50%, at least 60%, at least 70%, at least 80%, at least 90%, or at least 95% sequence identity to any amino acid sequence shown in SEQ ID NOs: 5-13. In some cases, an engineered microbial cell may express trpB and/or trpA that has been modified or mutated to exhibit higher activity levels.

In some cases, the microbial cell may be further engineered to express or overexpress one or more additional genes. In some aspects, such microbial cell may further express a tryptamine decarboxylase (see, e.g., Scheme 4, “decarboxylase”). In some cases, the tryptamine decarboxylase may be expressed by genomic integration of DNA or expression of a plasmid in the microbial cell. Tryptamine decarboxylases may be pyridoxal phosphate (PLP)-independent or may be PLP-dependent. In some cases, a tryptamine decarboxylase may comprise any one of the amino acid sequences according to SEQ ID NOs: 14-20 (see Table 2), or an amino acid sequence having at least 40%, at least 50%, at least 60%, at least 70%, at least 80%, at least 90%, or at least 95% sequence identity to an amino acid sequence of any one of SEQ ID NOs: 14-20. In some cases, an engineered microbial cell may express a tryptamine decarboxylase that has been modified or mutated to exhibit higher activity levels.

In some cases, the R1 and R2 amino positions of the tryptamine or substituted tryptamines derived from fermentation can be modified by a transferase to yield, by non-limiting example, N-methyl, N,N-dimethyl, N-acetyl, or N-hydroxycinnamoyl functional groups. Thus, in some cases, an engineered microbial cell may further express or overexpress a transferase (see, e.g., Scheme 4). In some cases, a transferase may comprise any one of the amino acid sequences shown in SEQ ID NOs: 21-31 (see Table 2), or an amino acid sequence having at least 40%, at least 50%, at least 60%, at least 70%, at least 80%, at least 90%, or at least 95% sequence identity to an amino acid sequence of any one of SEQ ID NOs: 21-31. In some cases, an engineered microbial cell may express a transferase that has been modified or mutated to exhibit higher activity levels. In some cases, an additional transferase can be expressed, such as, but not limited to, a phosphotransferase (kinase), acetyl transferase, glucosyl transferase, or sulfotransferase, to further modify hydroxyls on the indole ring of the tryptamine. For example, such as when engineered cells are cultivated in the presence of 6-hydroxyanthranilate or 4-hydroxy indole to yield 4-hydroxy-N,N- dimethyltryptamine, a kinase can be expressed yielding the phosphate ester of 4-hydroxy-N,N-dimethyltryptamine, psilocybin. Suitable kinases may include, but are not limited to, an amino acid sequence shown in any one of SEQ ID NOs: 41-44 (see Table 2), or an amino acid sequence having at least 40%, at least 50%, at least 60%, at least 70%, at least 80%, at least 90%, or at least 95% sequence identity to an amino acid sequence of any one of SEQ ID NOs: 41-44.

In some cases, the substituted indole may be any one of 5-hydroxyindole, 4-hydroxyindole, 7-hydroxyindole, and 4-chloroindole, 5-bromoindole, and 4-fluoroindole. In some cases, the tryptamine may be any one of tryptamine, 5-hydroxytryptamine, 5-hydroxymethyltryptamine, 5-hydroxy-N,N-dimethyltryptamine, 5-phosphoryloxymethyltryptamine, 5-phosphoryloxy-N,N-dimethyltryptamine, 4-hydroxytryptamine, 4-hydroxy-N,N-dimethyltryptamine, 4-phosphoryloxytryptamine, 4-phosphoryloxy-N,N-tryptamine, 7-hydroxytryptamine, 7-phosphoryloxymethyltryptamine, 7-phosphoryloxy-N,N-dimethyltryptamine, 4-chloro-tryptamine, 4-chloro-N,N-dimethyltryptamine, 5-bromotryptamine, 5-bromo-methyltryptamine, 5-bromo-N-methyltryptamine, 5-bromo-N,N-dimethyltryptamine, N-acetyl-tryptamine, and 4-hydroxy-N-acetyl-tryptamine.

### Synthesis of 4-hydroxyl Substituted and/or R1 or R2 Amine Substituted Tryptamines From Tryptophan by Engineered Microbial Cells

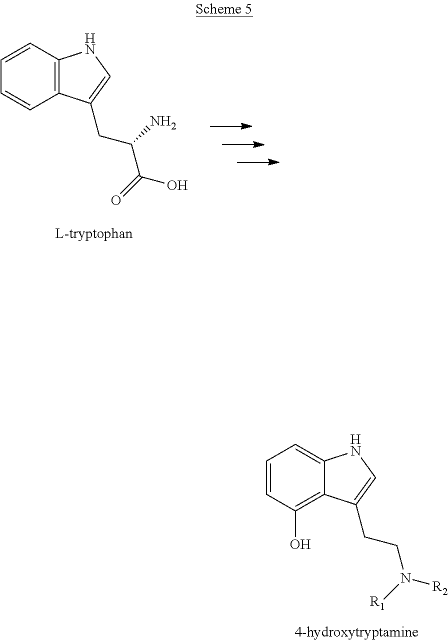

In another aspect, 4-hydroxyl substituted and/or R1 or R2 amine substituted tryptamines may be biosynthetically produced from tryptophan by engineered microbial cells, in accordance with Scheme 5.

In some cases, a microbial cell, may contain heterologous DNA on a plasmid or by integration into the genome that expresses enzymes that convert L-tryptophan to tryptamine (e.g., a decarboxylase) and/or that convert tryptamine to 4-hydroxytryptamine (e.g., a tryptophan 4-hydroxylase). Decarboxylases may be pyridoxal phosphate (PLP)-independent or PLP-dependent.

In some cases, the microbial cell may be engineered to express or overexpress a decarboxylase. In some cases, the decarboxylase may have an amino acid sequence of any one of SEQ ID NOs: 14-20 (see Table 2), or an amino acid sequence having at least 40%, at least 50%, at least 60%, at least 70%, at least 80%, at least 90%, or at least 95% sequence identity to an amino acid sequence of any one of SEQ ID NOs: 14-20. In some cases, an engineered microbial cell may express a decarboxylase that has been modified or mutated to exhibit higher activity levels.

In some cases, the microbial cell may be engineered to express or overexpress a tryptamine 4-hydroxylase. Tryptamine 4-hydroxylases are P450 enzymes that require a P450 reductase pair to provide reducing power via transfer of electrons from NADPH. In some cases, the tryptamine 4-hydroxylase may have an amino acid sequence according to any one of SEQ ID NOs: 32-35 (see Table 2), or an amino acid sequence having at least 40%, at least 50%, at least 60%, at least 70%, at least 80%, at least 90%, or at least 95% sequence identity to an amino acid sequence of any one of SEQ ID NOs: 32-35. In some cases, an engineered microbial cell may express a tryptamine 4-hydroxylase that has been modified or mutated to exhibit higher activity levels.

In some cases, the microbial cell may be engineered to express or overexpress a P450 reductase. In some cases, the P450 reductase may have an amino acid sequence according to any one of SEQ ID NOs: 36-40 (see Table 2), or an amino acid sequence having at least 40%, at least 50%, at least 60%, at least 70%, at least 80%, at least 90%, or at least 95% sequence identity to any one of SEQ ID NOs: 36-40. In some cases, an engineered microbial cell may express a P450 reductase that has been modified or mutated to exhibit higher activity levels.

In some cases, an additional transferase can be expressed, such as, but not limited to, a phosphotransferase (kinase), acetyl transferase, glucosyl transferase , or sulfotransferase to further modify hydroxyls on the indole ring of the tryptamine. When the production compound of interest is 4-hydroxy-N,N-dimethyltryptamine, a kinase can be expressed yielding the phosphate ester of 4-hydroxy-N,N-dimethyltryptamine, psilocybin. In some cases, the microbial cell may be further engineered to express or overexpress a kinase. In some cases, the kinase may have an amino acid sequence according to any one of SEQ ID NOs: 41-44 (see Table 2), or an amino acid sequence having at least 40%, at least 50%, at least 60%, at least 70%, at least 80%, at least 90%, or at least 95% sequence identity to any one of SEQ ID NOs: 41-44. In some cases, an engineered microbial cell may express a kinase that has been modified or mutated to exhibit higher activity levels.

### Synthesis of tryptamine derivatives from substitute tryptamines in engineered microbial cells

In another aspect, derivatives of tryptamine may be biosynthetically produced from substituted tryptamines by engineered microbial cells.

In some cases, a microbial cell, may contain heterologous DNA on a plasmid or by integration into the genome that expresses enzymes that convert a substitute tryptamine to a tryptamine derivative. In some cases, the microbial cell may be engineered to express or overexpress a tryptamine 4-hydroxylase. In some cases, the tryptamine 4-hydroxylase may have an amino acid sequence of any one of SEQ ID NOs: 32-35 (see Table 2), or an amino acid sequence having at least 40%, at least 50%, at least 60%, at least 70%, at least 80%, at least 90%, or at least 95% sequence identity to an amino acid sequence of any one of SEQ ID NOs: 32-35. In some cases, an engineered microbial cell may express a tryptamine 4-hydroxylase that has been modified or mutated to exhibit higher activity levels. In some cases, the microbial cell may be engineered to express or overexpress a tryptamine 5-hydroxylase. In some cases, the tryptamine 5-hydroxylase may have an amino acid sequence according to SEQ ID NO: 47 (see Table 2), or an amino acid sequence having at least 40%, at least 50%, at least 60%, at least 70%, at least 80%, at least 90%, or at least 95% sequence identity to an amino acid sequence according to SEQ ID NO: 47. In some cases, an engineered microbial cell may express a tryptamine 5-hydroxylase that has been modified or mutated to exhibit higher activity levels. In some cases, the microbial cell may be engineered to express or overexpress a 4-hydroxytryptamine kinase. In some cases, the 4-hydroxytryptamine kinase may have an amino acid sequence according to any one of SEQ ID NOs: 41-44 (see Table 2), or an amino acid sequence having at least 40%, at least 50%, at least 60%, at least 70%, at least 80%, at least 90%, or at least 95% sequence identity to an amino acid sequence according to any one of SEQ ID NOs: 41-44. In some cases, an engineered microbial cell may express a 4-hydroxytryptamine kinase that has been modified or mutated to exhibit higher activity levels.

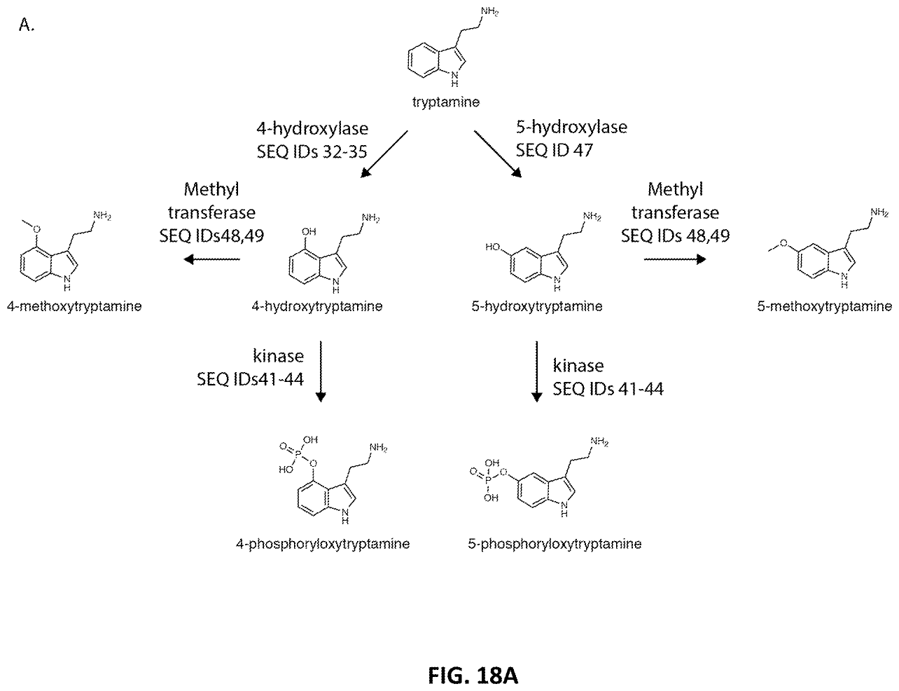

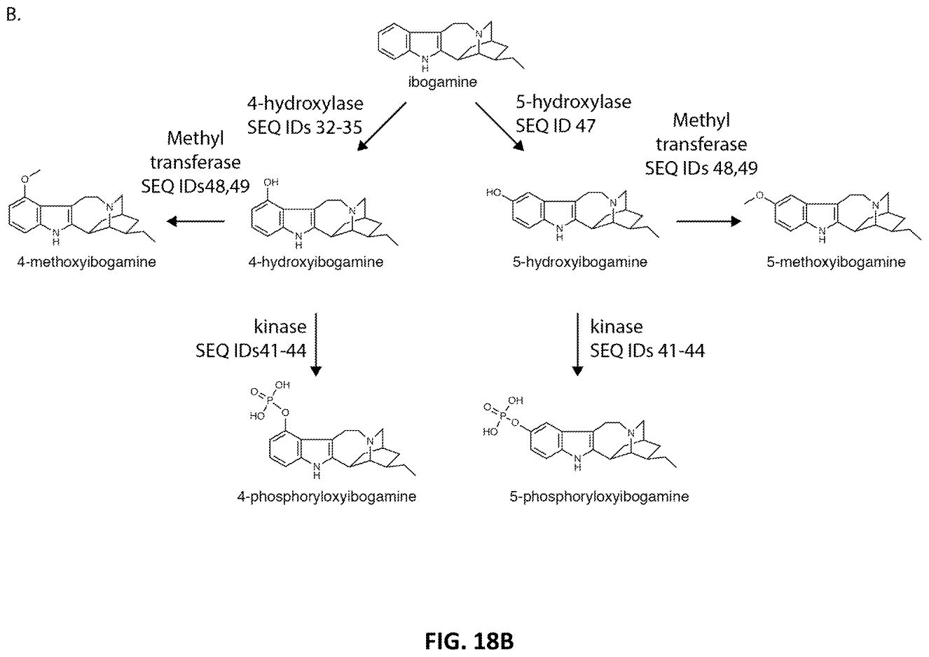

FIG. 18A depicts a non-limiting example of a biosynthetic pathway for converting tryptamine to tryptamine derivatives. For example, tryptamine may be converted to 4-hydroxytryptamine by 4-hydroxylase (e.g., SEQ ID NOs:32-35). 4-hydroxytryptamine may be converted to 4-methoxytryptamine by a methyl transferase (e.g., SEQ ID NOs:48 or 49), or 4-phosphoryloxytryptamine by a kinase (e.g., SEQ ID NOs:41-44). In another example, tryptamine may be converted to 5-hydroxytryptamine by 5-hydroxylase (e.g., SEQ ID NO:47). 5-hydroxytryptamine may be converted to 5-methoxytryptamine by a methyl transferase (e.g., SEQ ID NOs:48 or 49), or to 5-phosphoryloxytryptamine by a kinase (e.g., SEQ ID NOs:41-44).

FIG. 18B depicts a non-limiting example of a biosynthetic pathway converting ibogamine to derivatives of ibogamine. For example, ibogamine may be converted to 4-hydroxyibogamine by 4-hydroxylase (e.g., SEQ ID NOs:32-35). 4-hydroxyibogamine may be converted to 4-methoxyibogamine by a methyl transferase (e.g., SEQ ID NOs:48 or 49), or to 4-phosphoryloxyibogamine by a kinase (e.g., SEQ ID NOs:41-44). In another example, ibogamine may be converted to 5-hydroxyibogamine by 5-hydroxylase (e.g., SEQ ID NO:47). 5-hydroxyibogamine may be converted to 5-methoxyibogamine by a methyl transferase (e.g., SEQ ID NO:48 or 49), or to 5-phosphoryloxyibogamine by a kinase (e.g., SEQ ID NOs:41-44).

In some cases, the engineered microbial cell may be cultured in the presence of one or more tryptamines. In some cases, the tryptamine is selected from the group consisting of: 5-methoxy-N,N-dimethyl-tryptamine, N,N-diisopropyl-tryptamine, N-methyl-N-isopropyltryptamine, N,N-dimethyltryptamine, N,N-tetramethylenetryptamine, N,N-dipropyltryptamine, ibogamine, and 12-methoxyibogamine, tryptamine, 4-hydroxytryptamine, 5-hydroxytryptamine, ibogamine, 4-hydroxyibogamine, and 5-hydroxyibogamine.

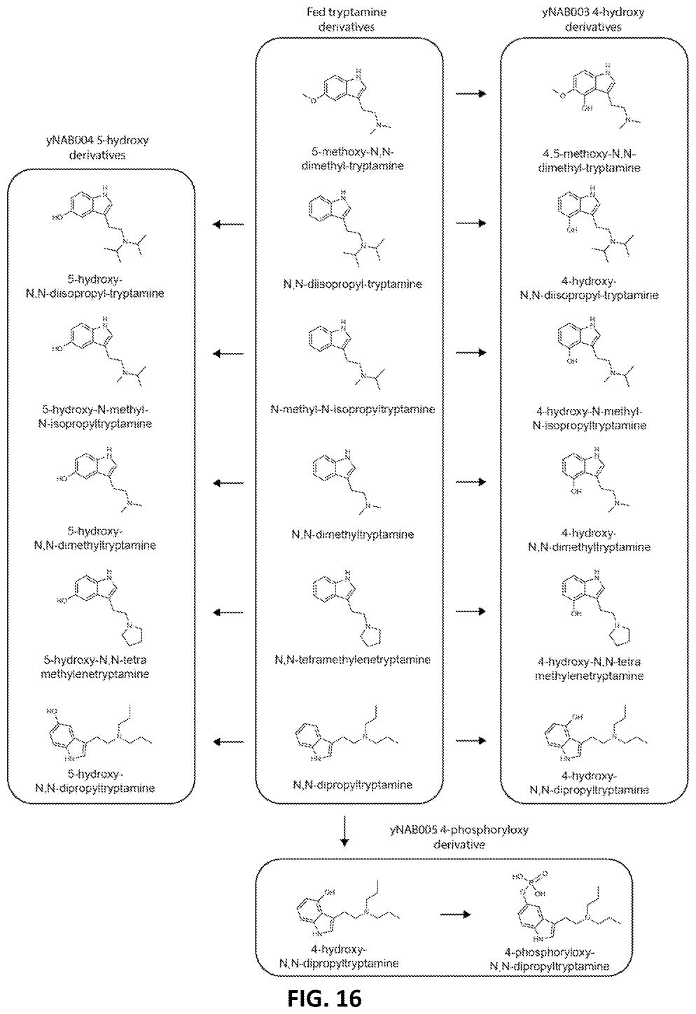

In some cases, the engineered microbial cell may convert a tryptamine to a tryptamine derivative. In some cases, the tryptamine derivative is any tryptamine derivative described in FIG. 16. In some cases, the tryptamine derivative is selected from the group consisting of: 5-hydroxy-N,N-diisopropyl-tryptamine, 5-hydroxy-N-methyl-N-isopropyltryptamine, 5-hydroxy-N,N-dimethyltryptamine, 5-hydroxy-N,N-tetramethylenetryptamine, 5-hydroxy-N,N-dipropyltryptamine, 4,5-methoxy-N,N-dimethyl-tryptamine, 4-hydroxy-N,N-diisopropyl-tryptamine, 4-hydroxy-N-methyl-N-isopropyltryptamine, 4-hydroxy-N,N-dimethyltryptamine, 4-hydroxy-N,N-tetramethylenetryptamine, 4-hydroxy-N,N-dipropyltryptamine, 4-phosphoryloxy-N,N-dipropyltryptamine, ibogamine, 12-methoxyibogamine, 4-hydroxytryptamine, 5-hydroxytryptamine, 4-methoxytryptamine, 5-methoxytryptamine, 4-phosphoryloxytryptamine, 5-phosphoryloxytryptamine, 4-hydroxyibogamine, 5-hydroxyibogamine, 4-phosphoryloxyibogamine, and 5-phosphoryloxyibogamine.

### Assay for Detecting Levels of 4-hydroxytryptamine in a Host Cell

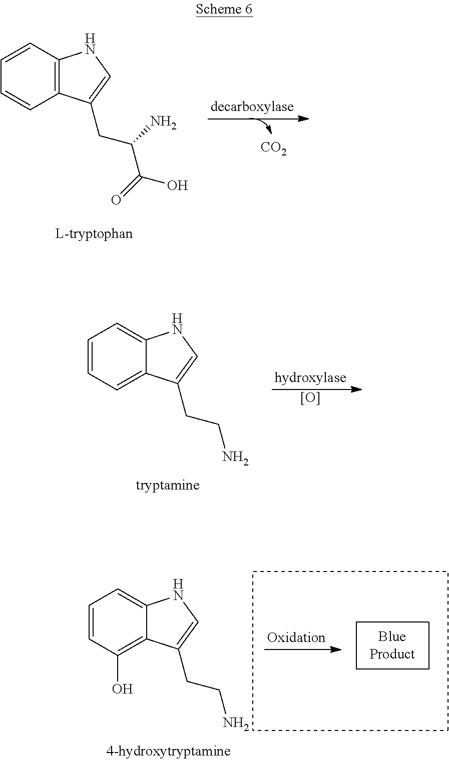

In another aspect, the disclosure provides a method for detecting levels of 4-hydroxytryptamine in a host cell. In some cases, the method comprises detecting, in a host cell genetically modified to produce a 4-hydroxytryptamine, a colored or fluorescent product of 4-hydroxytryptamine. In some cases, the colored or fluorescent product of 4-hydroxytryptamine may be produced by the action of an oxidizing mechanism produced in the cell. In some cases, the level of 4-hydroxytryptamine produced in the cell may be directly proportional to the level of 4-hydroxytryptamine or a colored product of 4-hydroxytryptamine produced in the cell (see, e.g., Scheme 6). Such in vivo screening methods may be used to rapidly screen for tryptamine 4-hydroxylase mutants having high activity in the engineered production host cell (DeLoache et al. 2015).

The oxidizing mechanism can be catalyzed by iron sulphate or by an enzyme expressed by a host cell, including, but not limited to, the enzyme multicopper oxidase (Blaschko and Levine 1960). A non-limiting example of a suitable oxidase is shown in SEQ ID NO: 45 (see Table 2). In some cases, a genetically modified cell comprising a nucleic acid sequence encoding a variant tryptophan 4-hydroxylase may produce a level of 4-hydroxytryptamine, or a colored or fluorescent product thereof, that is higher than a level of 4-hydroxytryptamine, or a colored or fluorescent product thereof, in a control cell not comprising a nucleic acid sequence encoding the variant tryptamine 4-hydroxylase. This may indicate that the variant enzyme increases flux through the biosynthetic pathway, thereby creating higher titers and rates of 4-hydroxytryptamine production. The genetically modified host cell containing higher 4-hydroxytryptamine production can contain enzymes, such as methyl-, sulphono-, glucosyl- and/or phospho transferases for the 4-hydroxyindole position or amino position, as described herein.

In some cases, the modified host cell may be modified to increase flux through tryptophan and to increase tryptophan production. This can be achieved by knockout of aro8 and aro10 and overexpression of TRP1, TRP2, TRP3, TRP4 and TRPS. Additionally, inclusion of TRP2 feedback resistant mutant allele can be employed.

In a non-limiting example (see Example 1), 1-tryptophan may be converted, in a modified microbial host cell expressing a decarboxylase, hydroxylase, P450 reductase, methyltransferase and kinase, to O-phosphoryl-4-hydroxy-N,N-dimethyltryptamine.

### Culture Conditions and Product Production

In some cases, the genetically modified host cell may be cultured under aerobic conditions. In some cases, the genetically modified host cell may be cultured under anaerobic conditions.

In some cases, the culture media may be a minimal media, including, but not limited to, M9, MOPS, YNB, ammonia salts, or a complex media containing, for example, yeast extract, casamino acids, peptone, or tryptone. In some cases, the culture media may be buffered, for example, by phosphate salts, HEPES, or Tris. In some cases, the culture media may contain a reducing agent, for example, L-ascorbic acid, dithiothreitol, or mercaptoethanol. In some cases, the culture media may be supplemented with additional amino acids, such as L-methionine, Histidine, Arginine, Alanine, Isoleucine, Cysteine, Aspartic acid, Leucine, Glutamine, Asparagine, Lysine, Glycine, Glutamic acid, Proline, Serine, Phenylalanine, Tyrosine, Selenocysteine, Threonine, Pyrrolysine, Tryptophan, or Valine. In some cases, additional vitamins and cofactors may be added, for example, L-ascorbic acid, thiamine, pyridoxal phosphate, niacin, pyridoxine, biotin, folic acid, tetrahydrofolic acid, riboflavin, pantothenic acid, copper salts, magnesium salts, manganese salts, molybdenum salts, iron salts, zinc salts, nickel salts, glutathione, heme, or D-aminolevulinic acid.

In some cases, the genetically modified host cell may be fed a substituted anthranilate by single addition, batch feeding, or constant dilution in culture. In some cases, the genetically modified host cell may be fed a substituted indole by single addition, batch feeding, or constant dilution in culture.

In some cases, a downstream product may be produced. In some cases, the downstream product may be purified, e.g., isolated and purified from the culture medium, from a cell lysate, or both. In some cases, the downstream product may be at least, or about, 25%, 30%, 40%, 50%, 60%, 70%, 75%, 80%, 90%, 95%, or 99%, by weight, pure. Purification can be carried out by any known method or combination of methods, which methods include, e.g., column chromatography, phase separation, precipitation, crystallization, decantation, gas stripping, membrane enhanced separation, fractionation, adsorption/desorption, pervaporation, thermal or vacuum desorption from a solid phase, extraction of the product that is immobilized or absorbed to a solid phase with a solvent, etc. Purity can be assessed by any appropriate method, e.g., by column chromatography, high performance liquid chromatography (HPLC) analysis, or gas chromatography-mass spectrometry (GC-MS) analysis.

In some cases, the cells in culture may convert greater than or about 0.0015, 0.002, 0.005, 0.01, 0.02, 0.05, 0.1, 0.12, 0.14, 0.16, 0.2, 0.3, 0.4, 0.5, 0.6, 0.7, 0.8, 0.9, 1.0, 1.2, 1.4, 1.6, 1.8, 2.0, 2.5, 3.0, 3.5, 4.0, 5.0, 6.0, 7.0, or 8.0% of the fed precursor in the cell culture medium into the desired product. In some cases, the cells in culture may produce at least 2 g/L, at least 3 g/L, at least 4 g/L, at least 5 g/L, at least 7 g/L, at least 10 g/L, or more than 50 g/L of the desired product in liquid culture medium.

In some cases, the cells in culture may convert greater than or about 0.0015, 0.002, 0.005, 0.01, 0.02, 0.05, 0.1, 0.12, 0.14, 0.16, 0.2, 0.3, 0.4, 0.5, 0.6, 0.7, 0.8, 0.9, 1.0, 1.2, 1.4, 1.6, 1.8, 2.0, 2.5, 3.0, 3.5, 4.0, 5.0, 6.0, 7.0, or 8.0% of the carbon in the cell culture medium into the desired product. In some cases, the cells in culture may produce at least 2 g/L, at least 3 g/L, at least 4 g/L, at least 5 g/L, at least 7 g/L, at least 10 g/L, or more than 50 g/L of the desired product in liquid culture medium.

### Host cells

Suitable host cells include cells that can be cultured in media, e.g., as unicellular organisms. Suitable host cells include yeast cells, fungal cells, insect cells, mammalian cells, algal cells, and bacterial cells. Suitable host cells may further include filamentous fungal cells; suitable filamentous fungal cells include, e.g., Aspergillus, Neurospora, and the like.

The host cell can be a prokaryotic cell. Suitable prokaryotic cells include, but are not limited to, any of a variety of laboratory strains of Escherichia coli, Corynebacterium glutamicum, Lactobacillus sp., Salmonella sp., Shigella sp., Citrobacter, Enterobacter, Clostridium, Klebsiella, Aerobacter, and the like. See, e.g., Carrier et al. (1992) J. Immunol. 148:1176-1181; U.S. Pat. No. 6,447,784; and Sizemore et al. (1995) Science 270:299-302. Examples of Salmonella strains which can be employed in the present invention include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some cases, the host cell is Escherichia coli.

Non-limiting examples of suitable yeast host cells are strains selected from a cell of a species of Candida, Kluyveromyces, Saccharomyces, Schizosaccharomyces, Pichia, Hansenula, and Yarrowia. In some cases, the yeast host cell may be selected from the group consisting of: Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Schizosaccharomyces pombe, Saccharomyces uvarum, Pichia kluyveri, Yarrowia hpolytica, Candida utilis, Candida cacaoi, and Geotrichum fermentans. Other useful yeast host cells are Kluyveromyces lactis, Kluyveromyces fragilis, Hansenula polymorpha, Pichia pastoris, Yarrowia lipolytica, Schizosaccharomyces pombe, Ustilgo maylis, Candida maltose, Pichia guillermondii and Pichia methanoliol. Suitable yeast host cells may include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pljperi, Pichia stiptis, Pichia methanolica, Pichia sp., Saccharomyces cerevisiae, Saccharomyces sp., Hansenula polymorpha, and the like. In some cases, a yeast host cell may be Saccharomyces cerevisiae; e.g., a genetically modified cell of the present disclosure may be a genetically modified Saccharomyces cerevisiae cell.

The filamentous fungi may be characterized by a mycelial wall composed of chitin, cellulose, glucan, chitosan, mannan, and other complex polysaccharides. Vegetative growth may be by hyphal elongation and carbon catabolism may be obligately aerobic. Suitable filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mucor, Myceliophthora, Neocallimastix, Neurospora, Paecilomyces, Penicillium, Piromyces, Phanerochaete, Pleurotus, Schizophyllum, Talaromyces, Thermoascus, Thielavia, Tolypocladium, and Trichoderma. Non-limiting examples of suitable filamentous fungal cells include, e.g., Aspergillus niger, Aspergillus awamori, Aspergillus foetidus, Aspergillus sojae, Aspergillus fumigatus, and Aspergillus oryzae. Another example of a suitable fungal cell is a Neurospora crassa cell.

### Heterologous Protein Expression in Modified Host Cells

In some cases, a nucleotide sequence encoding a heterologous polypeptide may be operably linked to a transcriptional control element.

Suitable promoters for expression in bacteria may include, but are not limited to, pT7, ptac, pLac, pLacUV5, pTet, pBAD, and the constitutive BBa series of promoters of the Anderson promoter library (Kelly et al, “Measuring the activity of BioBrick promoters using an in vivo reference standard” Journal of Biological Engineering 2009 3:4). Suitable promoters for expression in yeast may include, but are not limited to, TDH3, CCW12, CYC1, HIS3, GAL1, GAL10, ADH1, PGK, PHO5, GAPDH, ADC1, TRP1, URA3, LEU2, ENO, and TP1; and, AOX1 (e.g., for use in Pichia).

The expression vector may also contain a ribosome binding site for translation initiation and a transcription terminator. The expression vector may also include appropriate sequences for amplifying expression.

In some cases, the expression of the amino acid sequence may be codon optimized or biased to increase expression of protein in vivo. This may be achieved by several algorithms (Hanson and Coller, Nature Reviews Molecular Cell Biology volume 19, pages 20-30 (2018)), (Quax, et al Molecular Cell Review volume 59, Jul. 16, 2015). In some cases, the native amino acid sequence may be used for coding an amino acid sequence in vivo.

In some cases, a genetically modified microbial cell of the disclosure may comprise one or more nucleic acid sequences according to any one of SEQ ID NOs: 1-4 (see Table 1), or a nucleic acid sequence having at least 40%, at least 50%, at least 60%, at least 70%, at least 80%, at least 90%, or at least 95% sequence identity to any one of SEQ ID NOs: 1-4.

In some cases, a genetically modified microbial cell of the disclosure may express or overexpress one or more enzymes having an amino acid sequence according to any one of SEQ ID NOs: 5-49, or an amino acid sequence having at least 40%, at least 50%, at least 60%, at least 70%, at least 80%, at least 90%, or at least 95% sequence identity to any one of SEQ ID NOs: 5-49.

TABLE 1
DNA Sequences

| Oper- | | |
|---|---|---|
| onic | Host/ | |
| se- | Sequence | |
| quences | origin | Sequence |
| trpDCBA | Prokary- | atggctgacattctgctgctcgataatatcgactcttttacgtacaacctggcagatcagttgcg |
| | otic/ | cagcaatgggcataacgtggtgatttaccgcaaccatattccggcgcaaaccttaattgaacgcc |
| | E. coli | tggcgaccatgagcaatccggtgctgatgctttctcctggccccggtgtgccgagcgaagccggt |
| | | tgtatgccggaactcctcacccgcttgcgtggcaagctgcccattattggcatttgcctcggaca |
| | | tcaggcgattgtcgaagcttacgggggctatgtcggtcaggcgggcgaaattctccacggtaaag |
| | | cctccagcattgaacatgacggtcaggcgatgtttgccggattaacaaacccgctgccggtggcg |
| | | cgttatcactcgctggttggcagtaacattccggccggtttaaccatcaacgcccattttaatgg |
| | | catggtgatggcagtacgtcacgatgcggatcgcgtttgtggattccagttccatccggaatcca |
| | | ttctcaccacccagggcgctcgcctgctggaacaaacgctggcctgggcgcagcagaaactagag |
| | | ccagccaacacgctgcaaccgattctggaaaaactgtatcaggcgcagacgcttagccaacaag |
| | | aaagccaccagctgttttcagcggtggtgcgtggcgagctgaagccggaacaactggcggcg |
| | | gcgctggtgagcatgaaaattcgcggtgagcacccgaacgagatcgccggggcagcaaccgc |
| | | gctactggaaaacgcagcgccgttcccgcgcccggattatctgtttgctgatatcgtcggtactg |
| | | gcggtgacggcagcaacagtatcaatatttctaccgccagtgcgtttgtcgccgcggcctgtggg |
| | | ctgaaagtggcgaaacacggcaaccgtagcgtctccagtaaatctggttcgtccgatctgctggc |
| | | ggcgttcggtattaatcttgatatgaacgccgataaatcgcgccaggcgctggatgagttaggtg |
| | | tatgtttcctctttgcgccgaagtatcacaccggattccgccacgcgatgccggttcgccagcaa |
| | | ctgaaaacccgcaccctgttcaatgtgctggggccattgattaacccggcgcatccgccgctggc |
| | | gttaattggtgtttatagtccggaactggtgctgccgattgccgaaaccttgcgcgtgctggggt |
| | | atcaacgcgcggcggtggtgcacagcggcgggatggatgaagtttcattacacgcgccgacaatc |
| | | gttgccgaactgcatgacggcgaaattaaaagctatcagctcaccgcagaagactttggcctgac |
| | | accctaccaccaggagcaactggcaggcggaacaccggaagaaaaccgtgacattttaacacgt |
| | | ttgttacaaggtaaaggcgacgccgcccatgaagcagccgtcgctgcgaacgtcgccatgttaa |
| | | tgcgcctgcatggccatgaagatctgcaagccaatgcgcaaaccgttcttgaggtactgcgcagt |
| | | ggttccgcttacgacagagtcaccgcactggcggcacgagggtaaatgatgcaaaccgttttag |
| | | cgaaaatcgtcgcagacaaggcgatttgggtagaagcccgcaaacagcagcaaccgctggcc |
| | | agttttcagaatgaggttcagccgagcacgcgacatttttatgatgcgctacagggtgcgcgcac |
| | | ggcgtttattctggagtgcaagaaagcgtcgccgtcaaaaggcgtgatccgtgatgatttcgatc |
| | | cagcacgcattgccgccatttataaacattacgcttcggcaatttcggtgctgactgatgagaaa |
| | | tattttcaggggagctttaatttcctccccatcgtcagccaaatcgccccgcagccgattttatg |
| | | taaagacttcattatcgacccttaccagatctatctggcgcgctattaccaggccgatgcctgct |
| | | tattaatgctttcagtactggatgacgaccaatatcgccagcttgccgccgtcgctcacagtctg |
| | | gagatgggggtgctgaccgaagtcagtaatgaagaggaacaggagcgcgccattgcattgggagc |
| | | aaaggtcgttggcatcaacaaccgcgatctgcgtgatttgtcgattgatctcaaccgtacccgcg |
| | | agcttgcgccgaaactggggcacaacgtgacggtaatcagcgaatccggcatcaatacttacgct |
| | | caggtgcgcgagttaagccacttcgctaacggttttctgattggttcggcgttgatggcccatga |
| | | cgatttgcacgccgccgtgcgccgggtgttgctgggtgagaataaagtatgtggcctgacgcgtg |
| | | ggcaagatgctaaagcagcttatgacgcgggcgcgatttacggtgggttgatttttgttgcgaca |
| | | tcaccgcgttgcgtcaacgttgaacaggcgcaggaagtgatggctgcggcaccgttgcagtatgt |
| | | tggcgtgttccgcaatcacgatattgccgatgtggtggacaaagctaaggtgttatcgctggcgg |
| | | cagtgcaactgcatggtaatgaagaacagctgtatatcgatacgctgcgtgaagctctgccagca |
| | | catgttgccatctggaaagcattaagcgtcggtgaaaccctgcccgcccgcgagtttcagcacgt |
| | | tgataaatatgttttagacaacggccagggtggaagcgggcaacgttttgactggtcactattaa |
| | | atggtcaatcgcttggcaacgttctgctggcggggggcttaggcgcagataactgcgtggaagcg |
| | | gcacaaaccggctgcgccggacttgattttaattctgctgtagagtcgcaaccgggcatcaaaga |
| | | cgcacgtcttttggcctcggttttccagacgctgcgcgcatattaaggaaaggaacaatgacaac |
| | | attacttaacccctattttggtgagtttggcggcatgtacgtgccacaaatcctgatgcctgctc |
| | | tgcgccagctggaagaagcttttgtcagtgcgcaaaaagatcctgaatttcaggctcagttcaac |
| | | gacctgctgaaaaactatgccgggcgtccaaccgcgctgaccaaatgccagaacattacagccgg |
| | | gacgaacaccacgctgtatctcaagcgtgaagatttgctgcacggcggcgcgcataaaactaacc |
| | | aggtgctggggcaggcgttgctggcgaagcggatgggtaaaaccgaaatcatcgccgaaaccggt |
| | | gccggtcagcatggcgtggcgtcggcccttgccagcgccctgctcggcctgaaatgccgtattta |
| | | tatgggtgccaaagacgttgaacgccagtcgcctaacgtttttcgtatgcgcttaatgggtgcgg |
| | | aagtgatcccggtgcatagcggttccgcgacgctgaaagatgcctgtaacgaggcgctgcgcgac |
| | | tggtccggtagttacgaaaccgcgcactatatgctgggcaccgcagctggcccgcatccttatcc |
| | | gaccattgtgcgtgagtttcagcggatgattggcgaagaaaccaaagcgcagattctggaaagag |
| | | aaggtcgcctgccggatgccgttatcgcctgtgttggcggcggttcgaatgccatcggcatgttt |
| | | gctgatttcatcaatgaaaccaacgtcggcctgattggtgtggagccaggtggtcacggtatcga |
| | | aactggcgagcacggcgcaccgctaaaacatggtcgcgtgggtatctatttcggtatgaaagcgc |
| | | cgatgatgcaaaccgaagacgggcagattgaagaatcttactccatctccgccggactggatttc |
| | | ccgtctgtcggcccacaacacgcgtatcttaacagcactggacgcgctgattacgtgtctattac |
| | | cgatgatgaagcccttgaagccttcaaaacgctgtgcctgcacgaagggatcatcccggcgctgg |
| | | aatcctcccacgccctggcccatgcgttgaaaatgatgcgcgaaaacccggataaagagcagcta |
| | | ctggtggttaacctttccggtcgcggcgataaagacatcttcaccgttcacgatattttgaaagc |
| | | acgaggggaaatctgatggaacgctacgaatctctgtttgcccagttgaaggagcgcaaagaagg |
| | | cgcattcgttcctttcgtcacgctcggtgatccgggcattgagcagtcattgaaaattatcgata |
| | | cgctaattgaagccggtgctgacgcgctggagttaggtatccccttctccgacccactggcggat |
| | | ggcccgacgattcaaaacgccactctgcgcgcctttgcggcaggtgtgactccggcacaatgttt |
| | | tgaaatgctggcactgattcgccagaaacacccgaccattcccattggcctgttgatgtatgcca |
| | | atctggtgtttaacaaaggcattgatgagttttatgcccagtgcgaaaaagtcggcgtcgattcg |
| | | gtgctggttgccgatgtgccagttgaagagtccgcgcccttccgccaggccgcgttgcgtcataa |
| | | tgtcgcacctatcttcatctgcccgccaaatgccgatgacgacctgctgcgccagatagcctctt |
| | | acggtcgtggttacacctatttgctgtcacgagcaggcgtgaccggcgcagaaaaccgcgccgcg |
| | | ttacccctcaatcatctggttgcgaagctgaaagagtacaacgctgcacctccattgcagggatt |
| | | tggtatttccgccccggatcaggtaaaagcagcgattgatgcaggagctgcgggcgcgatttctg |
| | | gttcggccattgttaaaatcatcgagcaacatattaatgagccagagaaaatgctggcggcactg |
| | | aaagtttttgtacaaccgatgaaagcggcgacgcgcagttaa (SEQ ID NO: 1) |
| trpDCBA | Prokary- | atgaacagatttctacaattgtgcgttgacggaaaaacccttactgccggtgaggctgaaacgct |
| | otic/ | gatgaatatgatgatggcagcggaaatgactccttctgaaatgggggggatattgtcaattcttg |
| | B. | actctcggggggagacgccagaagagcttgcgggttttgtgaaggcaatgcgggcacacgctctt |
| | subtilis | acagtcgatggacttcctgatattgttgatacatgcggaacagggggagacggtatttccacttt |
| | | taatatctcaacggcctcggcaattgttgcctcggcagctggtgcgaaaatcgctaagcatggca |
| | | atcgctctgtctcttctaaaagcggaagcgctgatgttttagaggagctagaggtttctattcaa |
| | | accactcccgaaaaggtcaaaagcagcattgaaacaaacaacatgggatttctttttgcgccgct |
| | | ttaccattcgtctatgaaacatgtagcaggtactagaaaagagctaggtttcagaacggtattta |
| | | atctgcttgggccgctcagcaatcctttacaggcgaagcgtcaggtgattggggtctattctgtt |
| | | gaaaaagctggactgatggcaagcgcactggagacgtttcagccgaagcacgttatgtttgtatc |
| | | aagccgtgacggtttagatgagctttcaattacagcaccgaccgacgtgattgaattaaaggacg |
| | | gagagcgccgggagtataccgtttcacccgaagatttcggtttcacaaatggcagacttgaagat |
| | | ttacaggtgcagtctccgaaagagagcgcttatctcattcagaatatttttgaaaataaaagcag |
| | | cagttccgctttatctattacggcttttaatgcgggtgctgcgatttacacggcgggaattaccg |
| | | cctcactgaaggaaggaacggagctggcgttagagacgattacaagcggaggcgctgccgcgcag |
| | | cttgaacgactaaagcagaaagaggaagagatctatgcttgaaaaaatcatcaaacaaaagaaag |
| | | aagaagtgaaaacactggttctgccggtagagcagcctttcgagaaacgttcatttaaggaggcg |
| | | ctggcaagcccgaatcggtttatcgggttgattgccgaagtgaagaaagcatcgccgtcaaaagg |
| | | gcttattaaagaggattttgtacctgtgcagattgcaaaagactatgaggctgcgaaggcagatg |
| | | cgatttccgttttaacagacaccccgttttttcaaggggaaaacagctatttatcagacgtaaag |
| | | cgtgctgtttcgattcctgtacttagaaaagattttattgattctcttcaagtagaggaatcaag |
| | | aagaatcggagcggatgccatattgttaatcggcgaggtgcttgatcccttacaccttcatgaat |
| | | tatatcttgaagcaggtgaaaaggggatggacgtgttagtggaggttcatgatgcatcaacgcta |
| | | gaacaaatattgaaagtgttcacacccgacattctcggcgtaaataatcgaaacctaaaaacgtt |
| | | tgaaacatctgtaaagcagacagaacaaatcgcatctctcgttccgaaagaatccttgcttgtca |
| | | gcgaaagcggaatcggttctttagaacatttaacatttgtcaatgaacatggggcgcgagctgta |
| | | cttatcggtgaatcattgatgagacaaacttctcagcgtaaagcaatccatgctttgtttaggga |
| | | gtgaggttgtgaagaaaccggcattaaaatattgcggtattcggtcactaaaggatttgcagctt |
| | | gcggcggaatcacaggctgattacctaggatttatttttgctgaaagcaaacgaaaagtatctcc |
| | | ggaagatgtgaaaaaatggctgaaccaagttcgtgtcgaaaaacaggttgcaggtgtttttgtta |
| | | atgaatcaatagagacgatgtcacgtattgccaagagcttgaagctcgacgtcattcagcttcac |
| | | ggtgatgaaaaaccggcggatgctgctgctcttcgcaagctgacaggctgtgaaatatggaaggc |
| | | gcttcaccatcaagataacacaactcaagaaatagcccgctttaaagataatgttgacggctttg |
| | | tgattgattcatctgtaaaagggtctagaggcggaactggtgttgcattttcttgggaatgtgtg |
| | | ccggaatatcagcaggcggctattggtaaacgctgctttatcgctggcggcgtgaatccggatag |
| | | catcacacgcctattgaaatggcagccagaaggaattgaccttgccagcggaattgaaaaaaacg |
| | | gacaaaaagatcagaatctgatgaggcttttagaagaaaggatgaaccgatatgtatccatatcc |
| | | gaatgaaataggcagatacggtgattttggcggaaagtttgttccggaaacactcatgcagccgt |
| | | tagatgaaatacaaacagcatttaaacaaatcaaggatgatcccgcttttcgtgaagagtattat |
| | | aagctgttaaaggactattccggacgcccgactgcattaacatacgctgatcgagtcactgaata |
| | | cttaggcggcgcgaaaatctatttgaaacgagaagatttaaaccatacaggttctcataaaatca |
| | | ataatgcgctaggtcaagcgctgcttgctaaaaaaatgggcaaaacgaaaatcattgctgaaacc |
| | | ggtgccggccagcatggtgttgccgctgcaacagttgcagccaaattcggcttttcctgtactgt |
| | | gtttatgggtgaagaggatgttgcccgccagtctctgaacgttttccgcatgaagcttcttggag |
| | | cggaggtagtgcctgtaacaagcggaaacggaacattgaaggatgccacaaatgaggcgatccgg |
| | | tactgggttcagcattgtgaggatcacttttatatgattggatcagttgtcggcccgcatcctta |
| | | tccgcaagtggtccgtgaatttcaaaaaatgatcggagaggaagcgaaggatcagttgaaacgta |
| | | ttgaaggcactatgcctgataaagtagtggcatgtgtaggcggaggaagcaatgcgatgggtatg |
| | | tttcaggcatttttaaatgaagatgttgaactgatcggcgctgaagcagcaggaaaaggaattga |
| | | tacacctcttcatgccgccactatttcgaaaggaaccgtaggggttattcacggttcattgactt |
| | | atctcattcaggatgagttcgggcaaattattgagccctactctatttcagccggtctcgactat |
| | | cctggaatcggtccggagcatgcatatttgcataaaagcggccgtgtcacttatgacagtataac |
| | | cgatgaagaagcggtggatgcattaaagcttttgtcagaaaaagaggggattttgccggcaatcg |
| | | aatctgcccatgcgttagcgaaagcattcaaactcgccaaaggaatggatcgcggtcaactcatt |
| | | ctcgtctgtttatcaggccggggagacaaggatgtcaacacattaatgaatgtattggaagaaga |
| | | ggtgaaagcccatgtttaaattggatcttcaaccatcagaaaaattgtttatcccgtttattacg |
| | | gcgggcgatccagttcctgaggtttcgattgaactggcgaagtcactccaaaaagcaggcgccac |
| | | agcattggagcttggtgttgcatactctgacccgcttgcagacggtccggtgatccagcgggctt |
| | | caaagcgggcgcttgatcaaggaatgaatatcgtaaaggcaatcgaattaggcggagaaatgaaa |
| | | aaaaacggagtgaatattccgattatcctctttacgtattataatcctgtgttacaattgaacaa |
| | | agaatactttttcgctttactgcgggaaaatcatattgacggtctgcttgttccggatctgccat |
| | | tagaagaaagcaacagccttcaagaggaatgtaaaagccatgaggtgacgtatatttctttagtt |
| | | gcgccgacaagcgaaagccgtttgaaaaccattattgaacaagccgaggggttcgtctactgtgt |
| | | atcttctctgggtgtgaccggtgtccgcaatgagttcaattcatccgtgtacccgttcattcgta |
| | | ctgtgaagaatctcagcactgttccggttgctgtagggttcggtatatcaaaccgtgaacaggtc |
| | | ataaagatgaatgaaattagtgacggtgtcgtagtgggaagtgcgctcgtcagaaaaatagaaga |
| | | attaaaggaccggctcatcagcgctgaaacgagaaatcaggcgctgcaggagtttgaggattatg |
| | | caatggcgtttagcggcttgtacagtttaaaatga (SEQ ID NO: 2) |
| trpDCBA | Prokary- | atgaaaacaaggagtcatcaaatgaaaaatgaacttgaaaaagtgatgtcaggtcgtgacatgac |
| | otic/ | cgaaaatgaaatgaatatgcttgctaattcaattatccaaggtgaattaagcgaggtccaaattg |
| | L. | ccagctttttagtagcattaaaaatgaaaggtgaagcagcaagcgaattgactggtttggctcga |
| | lactis | gctttacaaaaagcagcgattcccattccaacaaatttgacaaatgcgatggacaattgtggaac |
| | | aggaggcgaccgctcattcagttttaatatttcaaccacagccgcttttgttttagcagctggtg |
| | | gagtcaatatggcaaaacacggaaatcgctccattaccagtaaatctggctcggcagacgttctt |
| | | gaggccttaggaatcaatctttatttaccagcagaaaagttagctcaagtttttgacaaagttgg |
| | | tttagttttcctttttgctcaaaatctccacccagcgatgaaatacttcacgccagtccgcagac |
| | | aactcgaaattccaacaattatgaacttgactgggccactaatcaatccaattccacttgatacg |
| | | caacttcttggtacctcacgtccagatttacttgaattaacagcaaatgttttgaaaggcttggg |
| | | ccgtaagcgagcattagtcatcacaggtgaaggcggaatggacgaagcaactccctttggactta |
| | | atcattacgcacttttagaaaatgacaaagtgactttgcatgaatttagagcctcagaagttggt |
| | | atttcagaagttcaactcaatgatattcgtggaggtgaagccccagaaaatgctgaaattttaaa |
| | | aaatgtccttgaaaatcaaccgtcagcctttttagaaacgaccgttttaaatgccggacttggat |
| | | tttatgccaatggaaaagttgattccatcaaatccggagttgaccttgcaagagaagtaattagt |
| | | acaggagcagctcttaccaagttgcatgaattacaagcagaacaaattggttaaaaatcttgatg |
| | | gcaaattttgaaatagcagaaaatgagagaaaaatatgaacataaaaaaaggaaaatttctagaa |
| | | acaatcctagcagaaaaacgacttgaaattgctaaaatgccagaagaacaagtaggaaaagttcg |
| | | tcaaacatacaatttttatgattacttaaaagaacattccgaccagcttcaagtgattgccgaag |
| | | tcaaaaaagcttcgcccagtctaggtgatattaacttagaagtggatatcgttgaccaagccaaa |
| | | aattacgaacaagccggtgccgctatgatttccgtcttaactgaccctgtattttttaaaggaaa |
| | | tattgaatatctctgtgaaatttcagaaaatgtccaaatccccaccttgaacaaggattttatca |
| | | tcgataaaaaacaaatcaatcgggcagttaatgcgggagcaacagttattttactcattgtcgca |
| | | gtttttgaaaatcaataccccaaactccaaaacctctacaactacgcactttcactaggacttga |
| | | agttcttgttgaaacacataataaagcagaacttgagattgctcatcagcttggagctaaaatta |
| | | ttggagttaataatcgtaatttaaaaacctttgaagtgatcttacaaaattcagtagatttgaca |
| | | ccctactttaaagaagacagtatctacatttccgaatcaggcatttttagcgcaaacgaagccca |
| | | aaaagtttccgatactttcaatggaatattggttggaacagcactcatgcaatcagaaaatctag |
| | | aaaaatctttgaaagatttaaaagtcaagaggaaaacgaatgaaaattaaaatctgtggcttatc |
| | | tacaaaagaagctgttgatacagctgtagaatctggtgtcacacatctcggttttattcttagtc |
| | | cctcaaaacgccaagttgcaccagaaaaaattcttcaaatcacaaacgatgtcccaaaaacagtc |
| | | aaaaaagtaggagtttttgttgatgaacctattgattttgtaaaaaaagccattcaagttgctca |
| | | actcgatctggttcagcttcacggaaatgaagatatgaattacattaatcaactagatatttcgg |
| | | ttattaaagcaataagaccagaccaagaatttaaagaatacgaagatgtaattttattatttgat |
| | | agtccacaagctggaagtggtcaagcatttgattgggactctttggtgaccagcggtctgaaaaa |
| | | taaatttttcatcgctggtggacttaatccagaaaatgtagcagctgctattcaacattttccaa |
| | | atgcctacggtgtggatgtttcttctggagtagaaactgacggaattaaaaaccttacaaaaata |
| | | aaaaactttgttcaaaatgcaagccttgcctcatcaaagcaattatttatagaatttttaagaat |
| | | cacaaaaaagctaaatgaaaataagattatcccttatttaatgggaagtttagcagttgagcaaa |
| | | taatcaattttccaacaaatcctgatgacattgatattcaactcaaaacgtctgattttgaaaat |
| | | tttgagcaattaacaagtttaatggaaaaattaggttatcagcttattgacttacatgagcataa |
| | | atttgaaaaagctagtattcatgttggctttgcaagtgtggagacccttaaaaactatgccgggg |
| | | ttgactatttgaccattcaacaagaaagaatggaaaatggcgaaaaatatcatcttccaaatgtt |
| | | gaacaatcccttaaaatctatgaggcagcaaaacgagatgagtggcgaggagggaagcaaaaaga |
| | | ttcctttattttcgatgagttaataaaggaacagaagaggaatgacaatgaatgataatcttatt |
| | | gaagagggtgtagagattcgaaatggtctcattattaagtcaattcaaaaagaagatatattaga |
| | | gctttggcaaattagttatggacctaaatctgatttacattggatgtctttcaacgctccctatt |
| | | ttgaggagccaatcctgagttgggaagaattttcaagaaaaatatctcttaaaataaattaacca |
| | | aatgttgcacttattatctttcaaaatcgaatcattggaatgctgtcagcttattgggaagacgg |
| | | taaattacaaaaatggcttgagtttggtatagtgatttatgatagtaaattgtggggacgtggaa |
| | | ttggacaggatgccttatctttttggttgaagcacctttttgaaacttatccgaagattcagcac |
| | | ataggatttacaacttggtcaggaaatcaaggaatgatgagactaggagaaaaaagtggtctaaa |
| | | acttgaagggcaaatcagaaaagttagatattggcaagaaacttggtatgattcaataaaatatg |
| | | gaattttaagagaagaactaaaaaaataaataaaaaaaatcaaaggagcaacaacatgacctaca |
| | | accaacctaacaacaaaggattttacggccaattcgggggccaattcgtacctgagacactaatg |
| | | acagcagtaaaacaattagaagaagcctacgtagatagtaaaaaagaccctctctttcaagcaga |
| | | acttaaagaattacttaaagactatgttggacgagaaaacccactctattatgcaaaacgcttaa |
| | | cagaatatgcgggcggagcaaaaatttatcttaaaagagaagacctaaaccatacaggagcacac |
| | | aaaattaacaatgccctcggacaagtcctccttgccaaaaaaatgggaaaaaataaagtcattgc |
| | | tgaaacaggtgcaggccaacacggtgtcgcaagcgcaaccgcggctgccctctttggcatggaat |
| | | gtacgatttatatgggtgaagaagacgttaaaagacaatctctcaatgtctttcgcatggaatta |
| | | ctcggggcaaaagttcattcagtaactgatggttcacgcgtacttaaagatgcggttaatgcagc |
| | | acttagagcatgggttgctcaagttgaagatacgcattatgtaatgggctcagttcttggaccac |
| | | atccatttccacaaattgtgcgtgattatcaagctgttattggacaggaagcgcgtgcccaattt |
| | | ttagaaaaagaaaataaacttccagatgctttagtagcttgtgtcggtggaggttcaaattctat |
| | | gggacttttttatcccttcgttaatgatgaatcagttgccatgtatggtgttgaagccgctggcc |
| | | ttgggattgatacaccacatcatgcggcaacaattactaaaggccgccccggtgttcttcacgga |
| | | acactcatggatgtccttcaagatgaaaatggtcaaatgttagaagcctttagtatttcagccgg |
| | | tttagactatccaggaatcggaccagaacactcttatttcaatgctgttggacgagcaaaatatg |
| | | ttgatattacagatgaagaagcacttgaaggttttaaaatcttatctagaactgaaggaattatc |
| | | ccagcactagaaagttctcatgctatcgcctatgcagtcaaattagcaaaagaattaggagcaga |
| | | taaatcaatgattgtttgtctttcaggacgtggagataaggatgtggttcaagttaaagaacgac |
| | | ttgaagcagaaaaagaggtgaaaaaatgaaaactttacaaatgactttaagcaataaaaaaaata |
| | | attttattccttatatcatggctggcgaccatgaaaaaggcttagaaggtcttaaagaaaccatt |
| | | caactgcttgagcaagctgggagttccgctattgaaattggcgttccattttcagatccggttgc |
| | | tgatggtccagtcatcgaacaagcaggtttgcgtgcgttagcaagaaatgtatcactttcaagta |
| | | ttcttgaaaccttaaaaacaattgatacaaaagttcctctagtaattatgacctatttcaatccc |
| | | gtttatcagtttggaattgaaaagtttgttgcagctcttgaaaaaacaccagttaaaggccttat |
| | | cattcctgatttgcctaaagaacatgaggactatatcaaaccatttatcaatgataaagatatct |
| | | gtttagttcctctggtctcattaaccacgccactttctcggcaaaaagaacttgtagccgatgct |
| | | gaaggatttatctatgccgttgcaataaatggagtaactgggaaagaaaatgcttatagtaacca |
| | | gcttgaccaacatttaaaagcgttatcttcattaacggatgttcctgttttgacaggatttggaa |
| | | tttctacattatctgatgtggaccgttttaataaagtgtcctcaggagttattgttggttcaaaa |
| | | attgttcgtgatttacatgaaggtaaagaaaacgaagttattaaatttattgaaaacgcaatcaa |
| | | tttttaa (SEQ ID NO: 3) |
| trpDCBA | Prokary- | atgacttctccagcaacactgaaagttctcaacgcctacttggataaccccactccaaccctgga |
| | otic/ | ggaggcaattgaggtgttcaccccgctgaccgtgggtgaatacgatgacgtgcacatcgcagcgc |
| | C. | tgcttgccaccatccgtactcgcggtgagcagttcgctgatattgccggcgctgccaaggcgttc |
| | glutamicum | ctcgcggcggctcgtccgttcccgattactggcgcaggtttgctagattccgctggtactggtgg |
| | | cgacggtgccaacaccatcaacatcaccaccggcgcatccctgatcgcagcatccggtggagtg |
| | | aagctggttaagcacggcaaccgttcggtgagctccaagtccggctccgccgatgtgctggaag |
| | | cgctgaatattcctttgggccttgatgtggatcgtgctgtgaagtggttcgaagcgtccaacttc |
| | | accttcctgttcgcacctgcgtacaaccctgcgattgcgcatgtgcagccggttcgccaggcgct |
| | | gaaattccccaccatcttcaacacgcttggaccattgctgtccccggcgcgcccggagcgtcaga |
| | | tcatgggcgtggccaatgccaatcatggacagctcatcgccgaggtcttccgcgagttgggccgt |
| | | acacgcgcgcttgttgtgcatggcgcaggcaccgatgagatcgcagtccacggcaccaccttggt |
| | | gtgggagcttaaagaagacggcaccatcgagcattacaccatcgagcctgaggaccttggcctt |
| | | ggccgctacacccttgaggatctcgtaggtggcctcggcactgagaacgccgaagctatgcgc |
| | | gctactttcgcgggcaccggccctgatgcacaccgtgatgcgttggctgcgtccgcaggtgcga |
| | | tgttctacctcaacggcgatgtcgactccttgaaagatggtgcacaaaaggcgctttccttgctt |
| | | gccgacggcaccacccaggcatggttggccaagcacgaagagatcgattactcagaaaaggagt |
| | | cttccaatgactagtaataatctgcccacagtgttggaaagcatcgtcgagggtcgtcgcggaca |
| | | cctggaggaaattcgcgctcgcatcgctcacgtggatgtggatgcgcttccaaaatccacccgtt |
| | | ctctgtttgattccctcaaccagggtaggggaggggcgcgtttcatcatggagtgcaagtccgca |
| | | tcgccttctttgggaatgattcgtgagcactaccagccgggtgaaatcgctcgcgtgtactctcg |
| | | ctacgccagcggcatttccgtgctgtgcgagccggatcgttttggtggcgattacgatcacctcg |
| | | ctaccgttgccgctacctctcatcttccggtgctgtgcaaagacttcatcattgatcctgtccag |
| | | gtacacgcggcgcgttactttggtgctgatgccatcctgctcatgctctctgtgcttgatgatga |
| | | agagtacgcagcactcgctgccgaggctgcgcgttttgatctggatatcctcaccgaggttattg |
| | | atgaggaggaagtcgcccgcgccatcaagctgggtgcgaagatctttggcgtcaaccaccgcaac |
| | | ctgcatgatctgtccattgatttggatcgttcacgtcgcctgtccaagctcattccagcagatgc |
| | | cgtgctcgtgtctgagtctggcgtgcgcgataccgaaaccgtccgccagctaggtgggcactcca |
| | | atgcattcctcgttggctcccagctgaccagccaggaaaacgtcgatctggcagcccgcgaatta |
| | | gtctacggccccaacaaagtctgcggactcacctcaccaagtgcagcacaaaccgctcgcgcagc |
| | | gggtgcggtctacggcgggctcatcttcgaagaggcatcgccacgcaatgtttcacgtgaaacat |
| | | tgcaaaaaatcatcgccgcagagcccaacctgcgctacgtcgcggtcagccgtcgcacctccggg |
| | | tacaaggatttgcttgtcgacggcatcttcgccgtacaaatccacgccccactgcaggacagcgt |
| | | cgaagcagaaaaggcattgatcgccgccgttcgtgaagaggttggaccgcaggtccaggtctggc |
| | | gcgcgatctcgatgtccagccccttgggggctgaagtggcagctgcggtggagggtgacgtcgat |
| | | aagctaattcttgatgcccatgaaggtggcagcggggaagtattcgactgggctacggtgccgg |
| | | ccgctgtgaaggcaaagtctttgctcgcgggaggcatctctccggacaacgctgcgcaggcact |
| | | cgctgtgggctgcgcaggtttggacatcaactctggcgtggaataccccgccggtgcaggcac |
| | | gtgggctggggcgaaagacgccggcgcgctgctgaaaattttcgcgaccatctccacattccatt |
| | | actaaaggtttaaataggatcatgactgaaaaagaaaacttgggcggctccacgctgctgcctgc |
| | | atacttcggtgaattcggcggccagttcgtcgcggaatccctcctgcctgctctcgaccagctgg |
| | | agaaggccttcgttgacgcgaccaacagcccagagttccgcgaagaactcggcggctacctcc |
| | | gcgattacctcggccgcccaaccccgctgaccgaatgctccaacctgccactcgcaggcgaag |
| | | gcaaaggctttgcgcggatcttcctcaagcgcgaagacctcgtccacggcggtgcacacaaaa |
| | | ctaaccaggtgatcggccaggtgctgcttgccaagcgcatgggcaaaacccgcatcatcgcag |
| | | agaccggcgcaggccagcacggcaccgccaccgctctcgcatgtgcgctcatgggcctcgag |
| | | tgcgttgtctacatgggcgccaaggacgttgcccgccagcagcccaacgtctaccgcatgcagc |
| | | tgcacggcgcgaaggtcatccccgtggaatctggttccggcaccctgaaggacgccgtgaatg |
| | | aagcgctgcgcgattggaccgcaaccttccacgagtcccactaccttctcggcaccgccgccg |
| | | gcccgcacccattcccaaccatcgtgcgtgaattccacaaggtgatctctgaggaagccaaggc |
| | | acagatgctagagcgcaccggcaagcttcccgacgttgtggtcgcctgtgtcggtggtggctcc |
| | | aacgccatcggcatgttcgcagacttcattgacgatgaaggtgtagagctcgtcggcgctgagc |
| | | cagccggtgaaggcctcgactccggcaagcacggcgcaaccatcaccaacggtcagatcggc |
| | | atcctgcacggcacccgttcctacctgatgcgcaactccgacggccaagtggaagagtcctact |
| | | ccatctccgccggacttgattacccaggcgtcggcccacagcacgcacacctgcacgccaccg |
| | | gccgcgccacctacgttggtatcaccgacgccgaagccctccaagcattccagtacctcgcccg |
| | | ctacgaaggcatcatccccgcactggaatcctcacacgcgttcgcctacgcactcaagcgcgcc |
| | | aagaccgccgaagaggaaggccagaacttaaccatcctcgtctccctatccggccgtggcgac |
| | | aaggacgttgaccacgtgcgccgcaccctcgaagaaaatccagaactgatcctgaaggacaac |
| | | cgatgagccgttacgacgatctttttgcacgcctcgacacggcaggggagggcgcctttgttccc |
| | | ttcatcatgctgagcgacccttcaccagaggaggctttccagatcatctccacagcaatcgaagc |
| | | tggcgcagatgcactggaacttggcgtacctttctccgacccagttgccgatggccccaccgtcg |
| | | cggaatcccacctccgcgcactcgacggcggcgccaccgtagacagcgcactcgagcagatc |
| | | aagcgcgtgcgcgcagcctacccagaggttcccatcggaatgctcatctacggcaacgttccttt |
| | | cacccgtggcttggatcgcttctaccaagagttcgctgaagctggcgcagactccatcctcctgc |
| | | cagacgtcccagtccgcgaaggcgcaccgttttctgcagcagctgcagcagccggaattgatcc |
| | | catttacatcgctccggccaacgccagcgagaaaaccctcgagggtgtctccgccgcatcaaag |
| | | ggctacatctacgccatctcccgcgacggcgtcaccggcaccgaacgtgaatcatccaccgac |
| | | ggcctgtccgcagtggtggacaacatcaagaaatttgatggcgcacccatcctcttgggcttcgg |
| | | catctcatcccctcagcacgtggcagacgcgattgcagcgggtgcttccggtgcgatcacgggt |
| | | tccgcgatcaccaagatcattgcttcccactgcgaaggtgagcacccgaacccgtccaccattcg |
| | | agatatggacggtttgaagaaggatctcactgagttcatctctgcgatgaaggcagcgaccaaga |
| | | aggtttag (SEQ ID NO: 4) |

TABLE 2
Enzymes

| Enzyme/ | genbank/ | |
|---|---|---|
| Source | uniprot | |
| Organism | number | Sequence |
| trpD/ | P00904 | MADILLLDNIDSFTYNLADQLRSNGHNVVIYRNHIPAQTLIER |
| E. coli | | LATMSNPVLMLSPGPGVPSEAGCMPELLTRLRGKLPIIGICLG |
| | | HQAIVEAYGGYVGQAGEILHGKASSIEHDGQAMFAGLTNPLP |
| | | VARYHSLVGSNIPAGLTINAHFNGMVMAVRHDADRVCGFQF |
| | | HPESILTTQGARLLEQTLAWAQQKLEPANTLQPILEKLYQAQT |
| | | LSQQESHQLFSAVVRGELKPEQLAAALVSMKIRGEHPNEIAG |
| | | AATALLENAAPFPRPDYLFADIVGTGGDGSNSINISTASAFVA |
| | | AACGLKVAKHGNRSVSSKSGSSDLLAAFGINLDMNADKSRQ |
| | | ALDELGVCFLFAPKYHTGFRHAMPVRQQLKTRTLFNVLGPLI |
| | | NPAHPPLALIGVYSPELVLPIAETLRVLGYQRAAVVHSGGMD |
| | | EVSLHAPTIVAELHDGEIKSYQLTAEDFGLTPYHQEQLAGGTP |
| | | EENRDILTRLLQGKGDAAHEAAVAANVAMLMRLHGHEDLQ |
| | | ANAQTVLEVLRSGSAYDRVTALAARG (SEQ ID NO: 5) |
| trpD/ | P06559 | MTSPATLKVLNAYLDNPTPTLEEAIEVFTPLTVGEYDDVHIAA |
| C. glutamicum | | LLATIRTRGEQFADIAGAAKAFLAAARPFPITGAGLLDSAGTG |
| | | GDGANTINITTGASLIAASGGVKLVKHGNRSVSSKSGSADVLE |
| | | ALNIPLGLDVDRAVKWFEASNFTFLFAPAYNPAIAHVQPVRQ |
| | | ALKFPTIFNTLGPLLSPARPERQIMGVANANHGQLIAEVFREL |
| | | GRTRALVVHGAGTDEIAVHGTTLVWELKEDGTIEHYTIEPED |
| | | LGLGRYTLEDLVGGLGTENAEAMRATFAGTGPDAHRDALAA |
| | | SAGAMFYLNGDVDSLKDGAQKALSLLADGTTQAWLAKHEEI |
| | | DYSEKESSND (SEQ ID NO: 6) |
| trpD* | P06559* | MTSPATLKVLNAYLDNPTPTLEEAIEVFTPLTVGEYDDVHIAA |
| feedback | | LLATIRTRGEQFADIAGAAKAFLAAARPFPITGAGLLDSAGTG |
| resistant | | GDGANTINITTGASLIAASGGVKLVKHGNRSVSSKSGSADVLE |
| (S149F, | | ALNIPLGLDVDRAVKWFEAFNFTFLFAPAYNPEIAHVQPVRQ |
| A161E)/C. | | ALKFPTIFNTLGPLLSPARPERQIMGVANANHGQLIAEVFREL |
| glutamicum | | GRTRALVVHGAGTDEIAVHGTTLVWELKEDGTIEHYTIEPED |
| | | LGLGRYTLEDLVGGLGTENAEAMRATFAGTGPDAHRDALAA |
| | | SAGAMFYLNGDVDSLKDGAQKALSLLADGTTQAWLAKHEEI |
| | | DYSEKESSND (SEQ ID NO: 7) |
| trpC/ | P00909 | MQTVLAKIVADKAIWVETRKEQQPLASFQNEVQPSTRHFYDA |
| E. coli | | LQGARTAFILECKKASPSKGVIRDDFDPARIAAIYKHYASAISV |
| | | LTDEKYFQGSFDFLPIVSQIAPQPILCKDFIIDPYQIYLARYYQA |
| | | DACLLMLSVLDDEQYRQLAAVAHSLEMGVLTEVSNEEELER |
| | | AIALGAKVVGINNRDLRDLSIDLNRTRELAPKLGHNVTVISES |
| | | GINTYAQVRELSHFANGFLIGSALMAHDDLNAAVRRVLLGEN |
| | | KVCGLTRGQDAKAAYDAGAIYGGLIFVATSPRCVNVEQAQE |
| | | VMAAAPLQYVGVFRNHDIADVADKAKVLSLAAVQLHGNED |
| | | QLYIDNLREALPAHVAIWKALSVGETLPARDFQHIDKYVFDN |
| | | GQGGSGQRFDWSLLNGQTLGNVLLAGGLGADNCVEAAQTG |
| | | CAGLDFNSAVESQPGIKDARLLASVFQTLRAY |
| | | (SEQ ID NO: 8) |
| trpC/ | P06560 | MTSNNLPTVLESIVEGRRGHLEEIRARIAHVDVDALPKSTRSL |
| C. glutamicum | | FDSLNQGRGGARFIMECKSASPSLGMIREHYQPGEIARVYSRY |
| | | ASGISVLCEPDRFGGDYDHLATVAATSHLPVLCKDFIIDPVQV |
| | | HAARYFGADAILLMLSVLDDEEYAALAAEAARFDLDILTEVI |
| | | DEEEVARAIKLGAKIFGVNHRNLHDLSIDLDRSRRLSKLIPAD |
| | | AVLVSESGVRDTETVRQLGGHSNAFLVGSQLTSQENVDLAAR |
| | | ELVYGPNKVCGLTSPSAAQTARAAGAVYGGLIFEEASPRNVS |
| | | RETLQKIIAAEPNLRYVAVSRRTSGYKDLLVDGIFAVQIHAPL |
| | | QDSVEAEKALIAAVREEVGPQVQVWRAISMSSPLGAEVAAA |
| | | VEGDVDKLILDAHEGGSGEVFDWATVPAAVKAKSLLAGGISP |
| | | DNAAQALAVGCAGLDINSGVEYPAGAGTWAGAKDAGALLK |
| | | IFATISTFHY (SEQ ID NO: 9) |
| trpB/ | P0A879 | MTTLLNPYFGEFGGMYVPQILMPALRQLEEAFVSAQKDPEFQ |
| E. coli | | AQFNDLLKNYAGRPTALTKCQNITAGTNTTLYLKREDLLHGG |
| | | AHKTNQVLGQALLAKRMGKTEIIAETGAGQHGVASALASAL |
| | | LGLKCRIYMGAKDVERQSPNVFRMRLMGAEVIPVHSGSATL |
| | | KDACNEALRDWSGSYETAHYMLGTAAGPHPYPTIVREFQRM |
| | | IGEETKAQILEREGRLPDAVIACVGGGSNAIGMFADFINETNV |
| | | GLIGVEPGGHGIETGEHGAPLKHGRVGIYFGMKAPMMQTED |
| | | GQIEESYSISAGLDFPSVGPQHAYLNSTGRADYVSITDDEALE |
| | | AFKTLCLHEGIIPALESSHALAHALKMMRENPDKEQLLVVNL |
| | | SGRGDKDIFTVHDILKARGEI (SEQ ID NO: 10) |
| trpB/ | P06561 | MTEKENLGGSTLLPAYFGEFGGQFVAESLLPALDQLEKAFVD |
| C. glutamicum | | ATNSPEFREELGGYLRDYLGRPTPLTECSNLPLAGEGKGFARI |
| | | FLKREDLVHGGAHKTNQVIGQVLLAKRMGKTRIIAETGAGQ |
| | | HGTATALACALMGLECVVYMGAKDVARQQPNVYRMQLHG |
| | | AKVIPVESGSGTLKDAVNEALRDWTATFHESHYLLGTAAGPH |
| | | PFPTIVREFHKVISEEAKAQMLERTGKLPDVVVACVGGGSNAI |
| | | GMFADFIDDEGVELVGAEPAGEGLDSGKHGATITNGQIGILH |
| | | GTRSYLMRNSDGQVEESYSISAGLDYPGVGPQHAHLHATGR |
| | | ATYVGITDAEALQAFQYLARYEGIIPALESSHAFAYALKRAKT |
| | | AEEEGQNLTILVSLSGRGDKDVDHVRRTLEENPELILKDNR |
| | | (SEQ ID NO: 11) |
| trpA/ | P00895 | MQTQKPTLELLTCEGAYRDNPTALFHQLCGDRPATLLLESAD |
| E. coli | | IDSKDDLKSLLLVDSALRITALGDTVTIQALSGNGEALLALLD |
| | | NALPAGVESEQSPNCRVLRFPPVSPLLDEDARLCSLSVFDAFR |
| | | LLQNLLNVPKEEREAMFFGGLFSYDLVAGFEDLPQLSAENNC |
| | | PDFCFYLAETLMVIDHQKKSTRIQASLFAPNEEEKQRLTARLN |
| | | ELRQQLTEAAPPLPVVSVPHMRCECNQSDEEFGGVVRLLQKA |
| | | IRAGEIFQVVPSRRFSLPCPSPLAAYYVLKKSNPSPYMFFMQD |
| | | NDFTLFGASPESSLKYDATSRQIEIYPIAGTRPRGRRADGSLDR |
| | | DLDSRIELEMRTDHKELSEHLMLVDLARNDLARICTPGSRYV |
| | | ADLTKVDRYSYVMHLVSRVVGELRHDLDALHAYRACMNM |
| | | GTLSGAPKVRAMQLIAEAEGRRRGSYGGAVGYFTAHGDLDT |
| | | CIVIRSALVENGIATVQAGAGVVLDSVPQSEADETRNKARAV |
| | | LRAIATAHHAQETF (SEQ ID NO: 12) |
| trpA/ | P06562 | MSRYDDLFARLDTAGEGAFVPFIMLSDPSPEEAFQIISTAIEAG |
| C. glutamicum | | ADALELGVPFSDPVADGPTVAESHLRALDGGATVDSALEQIK |
| | | RVRAAYPEVPIGMLIYGNVPFTRGLDRFYQEFAEAGADSILLP |
| | | DVPVREGAPFSAAAAAAGIDPIYIAPANASEKTLEGVSAASKG |
| | | YIYAISRDGVTGTERESSTDGLSAVVDNIKKFDGAPILLGFGIS |
| | | SPQHVADAIAAGASGAITGSAITKIIASHCEGEHPNPSTIRDMD |
| | | GLKKDLTEFISAMKAATKKV (SEQ ID NO: 13) |
| Tryptophan | P0DPA6 | MQVIPACNSAAIRSLCPTPESFRNMGWLSVSDAVYSEFIGELA |
| Decarboxylase/ | | TRASNRNYSNEFGLMQPIQEFKAFIESDPVVHQEFIDMFEGIQ |
| P. cubensis | | DSPRNYQELCNMFNDIFRKAPVYGDLGPPVYMIMAKLMNTR |
| | | AGFSAFTRQRLNLHFKKLFDTWGLFLSSKDSRNVLVADQFDD |
| | | RHCGWLNERALSAMVKHYNGRAFDEVFLCDKNAPYYGFNS |
| | | YDDFFNRRFRNRDIDRPVVGGVNNTTLISAACESLSYNVSYD |
| | | VQSLDTLVFKGETYSLKHLLNNDPFTPQFEHGSILQGFLNVTA |
| | | YHRWHAPVNGTIVKIINVPGTYFAQAPSTIGDPIPDNDYDPPP |
| | | YLKSLVYFSNIAARQIMFIEADNKEIGLIFLVFIGMTEISTCEAT |
| | | VSEGQHVNRGDDLGMFHFGGSSFALGLRKDCRAEIVEKFTEP |
| | | GTVIRINEVVAALKA (SEQ ID NO: 14) |
| Tryptophan | ASU62242 | MQVLPACQSSALKTLCPSPEAFRKLGWLPTSDEVYNEFIDDLT |
| Decarboxylase/ | | GRTCNEKYSSQVTLLKPIQDFKTFIENDPIVYQEFISMFEGIEQS |
| P. | | PTNYHELCNMFNDIFRKAPLYGDLGPPVYMIMARIMNTQAGF |
| cyanescens | | SAFTKESLNFHFKKLFDTWGLFLSSKNSRNVLVADQFDDKHY |
| | | GWFSERAKTAMMINYPGRTFEKVFICDEHVPYHGFTSYDDFF |
| | | NRRFRDKDTDRPVVGGVTDTTLIGAACESLSYNVSHNVQSLD |
| | | TLVIKGEAYSLKHLLHNDPFTPQFEHGSIIQGFLNVTAYHRWH |
| | | SPVNGTIVKIVNVPGTYFAQAPYTIGSPIPDNDRDPPPYLKSLV |
| | | YFSNIAARQIMFIEADNKDIGLIFLVFIGMTEISTCEATVCEGQH |
| | | VNRGDDLGMFHFGGSSFALGLRKDSKAKILEKFAKPGTVIRIN |
| | | ELVASVRK (SEQ ID NO: 15) |
| Tryptophan | PPQ80975 | MQVLTACYTSTLKSLLPSFDAFRSMGWLPVSDKTYNEWIGDL |
| Decarboxylase/ | | RSRASDKNYTSQVGLIQPIKDFKAFIESDPVVHQEFITMFEGIE |
| P. | | ESPRNYEELCHMFNDIFRKAPVYGDLGPPVYMVMARIMNTQ |
| cyanescens | | AGFSAFTKQSLNSHFKRLFDTWGVFLSSKESRYVLVTDQFDD |
| | | NHYGWLSDRAKSAMVKHYYGRTFEQVFICDEHAPYHGFQSY |
| | | DDFFNRRFRDRDIDRPVVGGIENTTLISAACESLSYNVCHDLQ |
| | | SLDTLFVKGESYSLKHLLNDDPFARQFEHGSILQGFLNVTAYH |
| | | RWHAPVNGTILKIINVPGTYFAQAPHTIGDSLDSDHPPYLKSL |
| | | AYFSNIAARQIMFIEADNKDIGLIFLVFIGMTEISTCEATVSEGQ |
| | | HVNRGDDLGMFHFGGSSFALGLRKDCKAEIFERFAEQGTVIKI |
| | | NEVVAAVKD (SEQ ID NO: 16) |
| Tryptophan | PPQ70875 | MAKTLRPTAQAFRELGWLPASDGVYNKFMKDLTNRASNEN |
| Decarboxylase/ | | HLCHVALLQPIQDFKTFIENDPVVYQEFVCMFEGIEESPRNYH |
| G. dilepis | | ELCNMFNEIFRRAPYYGDLGPPVYMAMAKIMNTRAGFSAFTR |
| | | ESLNFHFKRLFDTWGLFLSSPASRDVLVADKFDSKHYGWFSE |
| | | PAKAAMMAQYDGRTFEQVFICDETAPYHGFKSYDDFFNRKF |
| | | RAMDIDRPVVGGIANTTLIGSPCEALSYNVSDDVHSLETLYFK |
| | | GEGYSLRHLLHDDPSTEQFEHGSIIQGFLNITGYHRWHAPVSG |
| | | TIMKIVDVPGTYFAQAPSTIGDPFPVNDYDPQAPYLRSLAYFS |
| | | NIAARQIIFIQADNEDIGLIYLILIGMTEVSTCEALVCPGQHVER |
| | | GDDLGMFHFGGSSFALGLRKNSKAAILEELKTQGTVIKVNDV |
| | | IAAVQA (SEQ ID NO: 17) |
| Tryptophan | P20711 | MNASEFRRRGKEMVDYMANYMEGIEGRQVYPDVEPGYLRP |
| Decarboxylase/ | | LIPAAAPQEPDTFEDIINDVEKIIMPGVTHWHSPYFFAYFPTAS |
| H. sapiens | | SYPAMLADMLCGAIGCIGFSWAASPACTELETVMMDWLGK |
| | | MLELPKAFLNEKAGEGGGVIQGSASEATLVALLAARTKVIHR |
| | | LQAASPELTQAAIMEKLVAYSSDQAHSSVERAGLIGGVKLKA |
| | | IPSDGNFAMRASALQEALERDKAAGLIPFFMVATLGTTTCCSF |
| | | DNLLEVGPICNKEDIWLHVDAAYAGSAFICPEFRHLLNGVEFA |
| | | DSFNFNPHKWLLVNFDCSAMWVKKRTDLTGAFRLDPTYLKH |
| | | SHQDSGLITDYRHWQIPLGRRFRSLKMWFVFRMYGVKGLQA |
| | | YIRKHVQLSHEFESLVRQDPRFEICVEVILGLVCFRLKGSNKV |
| | | NEALLQRINSAKKIHLVPCHLRDKFVLRFAICSRTVESAHVQR |
| | | AWEHIKELAADVLRAERE (SEQ ID NO: 18) |
| Tryptophan | I0DFJ0 | MSENLQLSAEEMRQLGYQAVDLIIDHMNHLKSKPVSETIDSDI |
| Decarboxylase/ | | LRNKLTESIPENGSDPKELLHFLNRNVFNQITHVDHPHFLAFV |
| B. | | PGPNNYVGVVADFLASGFNVFPTAWIAGAGAEQIELTTINWL |
| atrophaeus | | KSMLGFPDSAEGLFVSGGSMANLTALTVARQAKLNNDIENA |
| | | VVYFSDQTHFSVDRALKVLGFKHHQICRIETDEHLRISVSALK |
| | | KQIKEDRTKGKKPFCVIANAGTTNCGAVDSLNELADLCNDED |
| | | VWLHADGSYGAPAILSEKGSAMLQGIHRADSLTLDPHKWLF |
| | | QPYDVGCVLIRNSQYLSKTFRMMPEYIKDSETNVEGEINFGEC |
| | | GIELSRRFRALKVWLSFKVFGVAAFRQAIDHGIMLAEQVEAF |
| | | LGKAKDWEVVTPAQLGIVTFRYIPSELASTDTINEINKKLVKEI |
| | | THRGFAMLSTTELKEKVVIRLCSINPRTTTEEMLQIMMKIKAL |
| | | AEEVSISYPCVAE (SEQ ID NO: 19) |
| Tryptophan | P17770 | MGSIDSTNVAMSNSPVGEFKPLEAEEFRKQAHRMVDFIADYY |
| Decarboxylase/ | | KNVETYPVLSEVEPGYLRKRIPETAPYLPEPLDDIMKDIQKDII |
| C. roseus | | PGMTNWMSPNFYAFFPATVSSAAFLGEMLSTALNSVGFTWV |
| | | SSPAATELEMIVMDWLAQILKLPKSFMFSGTGGGVIQNTTSES |
| | | ILCTIIAARERALEKLGPDSIGKLVCYGSDQTHTMFPKTCKLA |
| | | GIYPNNIRLIPTTVETDFGISPQVLRKMVEDDVAAGYVPLFLC |
| | | ATLGTTSTTATDPVDSLSEIANEFGIWIHVDAAYAGSACICPEF |
| | | RHYLDGIERVDSLSLSPHKWLLAYLDCTCLWVKQPHLLLRAL |
| | | TTNPEYLKNKQSDLDKVVDFKNWQIATGRKFRSLKLWLILRS |
| | | YGVVNLQSHIRSDVAMGKMFEEWVRSDSRFEIVVPRNFSLVC |
| | | FRLKPDVSSLHVEEVNKKLLDMLNSTGRVYMTHTIVGGIYML |
| | | RLAVGSSLTEEHHVRRVWDLIQKLTDDLLKEA |
| | | (SEQ ID NO: 20) |
| Tryptamine n- | ASU62238.1 | MHIRNPYRTPIDYQALSEAFPPLKPFVSVNADGTSSVDLTIPEA |
| methyltrans- | | QRAFTAALLHRDFGLTMTIPEDRLCPTVPNRLNYVLWIEDIFN |
| ferase/ P. | | YTNKTLGLSDDRPIKGVDIGTGASAIYPMLACARFKAWSMVG |
| cubensis | | TEVERKCIDTARLNVVANNLQDRLSILETSIDGPILVPIFEATEE |
| | | YEYEFTMCNPPFYDGAADMQTSDAAKGFGFGVGAPHSGTVI |
| | | EMSTEGGESAFVAQMVRESLKLRTRCRWYTSNLGKLKSLKEI |
| | | VGLLKELEISNYAINEYVQGSTRRYAVAWSFTDIQLPEELSRP |
| | | SNPELSSLF (SEQ ID NO: 21) |
| Tryptamine n- | PPQ83230.1 | MHIRNPYRSPIDYQALVEAFPPLRPYVTVNQDNTTSIDLTVPE |
| methyltrans- | | VQRLYTAALLHRDFGLVIDLPEDRLCPTLLTRTPRLNYVLWV |
| ferase/P. | | EDILKVTNTALGLSEDRPVKGIDIGTGAAAIYPMLACARFKTW |
| cyanescens | | SMIGTEIDRKCIDTARVNVLTNNLQDRLSIIETSIDGPILVPIFEA |
| | | TTDYEYDFTMCNPPFYDGAADMQTSDAAKGFGFGVNAPHSG |
| | | TVIEMSTEGGESAFVAQMVRESLDHRTRCRWFTSNLGKLKSL |
| | | HEIVGLLREHQISNYAINEYVQGTTRRYAIAWSFTNIRLPEDLT |
| | | RPSNPELSSLF (SEQ ID NO: 22) |
| Tryptamine n- | PPQ80976.1 | MHNRNPYRDVIDYQALAEAYPPLKPHVTVNADNTASIDLTIP |
| methyltrans- | | EVQRQYTAALLHRDFGLTITLPEDRLCPTVPNRLNYVLWIEDI |
| ferase/P. | | FQCTNKALGLSDDRPVKGVDIGTGASAIYPMLACARFKQWS |
| cyanescens | | MIATEVERKCIDTARLNVLANNLQDRLSILEVSVDGPILVPIFD |
| | | TFERATSDYEFEFTMCNPPFYDGAADMQTSDAAKGFGFGVN |
| | | APHSGTVIEMATEGGEAAFVAQMVRESMKLQTRCRWFTSNL |
| | | GKLKSLHEIVALLRESQITNYAINEYVQGTTRRYALAWSFTDI |
| | | KLTEELYRPSNPELGPLCSTFV (SEQ ID NO: 23) |
| Tryptamine n- | PPQ70884.1 | MHIRNPYLTPPDYEALAEAFPALKPYVTVNPDKTTTIDFAIPE |
| methyltrans- | | AQRLYTAALLYRDFGLTITLPPDRLCPTVPNRLNYVLWIQDIL |
| ferase/G. | | QITSAALGLPEARQVKGVDIGTGAAAIYPILGCSLAKNWSMV |
| dilepis | | GTEVEQKCIDIARQNVISNGLQDRITITANTIDAPILLPLFEGDS |
| | | NFEWEFTMCNPPFYDGAADMETSQDAKGFGFGVNAPHTGTV |
| | | VEMATDGGEAAFVSQMVRESLHLKTRCRWFTSNLGKLKSLH |
| | | EIVGLLREHQITNYAINEYVQGTTRRYAIAWSFTDLRLSDHLP |
| | | RPPNPDLSALF (SEQ ID NO: 24) |
| Tryptamine n- | O95050 | MKGGFTGGDEYQKHFLPRDYLATYYSFDGSPSPEAEMLKFNL |
| methyltrans- | | ECLHKTFGPGGLQGDTLIDIGSGPTIYQVLAACDSFQDITLSDF |
| ferase/H. | | TDRNREELEKWLKKEPGAYDWTPAVKFACELEGNSGRWEEK |
| sapiens | | EEKLRAAVKRVLKCDVHLGNPLAPAVLPLADCVLTLLAMEC |
| | | ACCSLDAYRAALCNLASLLKPGGHLVTTVTLRLPSYMVGKR |
| | | EFSCVALEKEEVEQAVLDAGFDIEQLLHSPQSYSVTNAANNG |
| | | VCFIVARKKPGP (SEQ ID NO: 25) |
| Tryptamine n- | WP_08616 | MIYKFYQQHIFPHLLNQVMQTPSLMDQRRQLLLPIAGDVLEIG |
| methyltrans- | 4675.1 | FGTGVNLPFYQNVETLYALEPNADLYQLAAKRIHESTIHVQHI |
| ferase/A. sp. | | QAYAEKLPFADASLDHIVSTWTLCSIENLAQALIEMYRVLKPN |
| ANC 4654 | | GTLHLVEHVQYQDNAKLQHLQNLLTPIQKRLADGCHLNRNIE |
| | | QALRDAHFDFTEQHYFAAQGIPKLAQRMFFARAQKQPE |
| | | (SEQ ID NO: 26) |
| Tryptamine | A0A1L2E | MEITSSAMLKTTTTPPHPLAGEKVPLSAFDRAAFDVFVPLVFA |
| benzoyl | H62 | YRAPAPSSEAVKEGLRVAVAAYPLVSGRIAVDGQGRRRRRR |
| transferase/ | | VLHVNNEGVLVLDATVEVDLDAVLAANVATDLYPALPEHSF |
| O. sativa | | GAALLQVQLTRFGCGGLVVGLIGHHHVFDGHSMSTFCATWA |
| subsp. | | RAVRDSEAFIVPSPSLDRAITGVPRSPPAPVFDHRSIEFKVGNK |
| japonica | | SSDSSGAAAAAAVEKIANIGVRFTAKFVAELKARVGGRCSTF |
| | | ECVLAHAWKKITAARGLKPEEFTRVRVAVNCRRRANPPAPA |
| | | DLFGNMVLWAFPRLQVRRLLSSSYRDVVGAIRAAVARVDAE |
| | | YIQSFVDYVEVADARGEELAATAAEPGETLCPDLEVDSWLGF |
| | | RFHEMDLGTGPPAAVLSPDLPIEGLMILVPVGGDGGGVDLFV |
| | | ALADDHAQAFEQICYSLEEHAMIHSHL |
| | | (SEQ ID NO: 27) |
| Serotonin N- | Q16613 | MSTQSTHPLKPEAPRLPPGIPESPSCQRRHTLPASEFRCLTPED |
| acetyltrans- | | AVSAFEIEREAFISVLGVCPLYLDEIRHFLTLCPELSLGWFEEG |
| ferase/H. | | CLVAFIIGSLWDKERLMQESLTLHRSGGHIAHLHVLAVHRAF |
| sapiens | | RQQGRGPILLWRYLHHLGSQPAVRRAALMCEDALVPFYERFS |
| | | FHAVGPCAITVGSLTFMELHCSLRGHPFLRRNSGC |
| | | (SEQ ID NO: 28) |
| Dopamine N- | Q94521 | MEVQKLPDQSLISSMMLDSRCGLNDLYPIARLTQKMEDALTV |
| acetyltrans- | | SGKPAACPVDQDCPYTIELIQPEDGEAVIAMLKTFFFKDEPLN |
| ferase/D. | | TFLDLGECKELEKYSLKPLPDNCSYKAVNKKGEIIGVFLNGL |
| melanogaster | | MRRPSPDDVPEKAADSCEHPKFKKILSLMDHVEEQFNIFDVYP |
| | | DEELILDGKILSVDTNYRGLGIAGRLTERAYEYMRENGINVYH |
| | | VLCSSHYSARVMEKLGFHEVFRMQFADYKPQGEVVFKPAAP |
| | | HVGIQVMAKEVGPAKAAQTKL (SEQ ID NO: 29) |
| Arylalkyla- | Q9PVD7 | MMAPQVVSSPFLKPFFLKTPISVSSPRRQRRHTLPASEFRNLTP |
| mine N- | | QDAISVFEIEREAFISVSGECPLTLDEVLVFLGQCPELSMGWFE |
| acetyltrans- | | EGQLVAFIIGSGWDKEKLEQEAMSTHVPDSPTVHIHVLSVHR |
| ferase/D. | | HCRQQGKGSILLWRYLQYLRCLPGLRRALLVCEEFLVPFYQK |
| rerio | | AGFKEKGPSAISVAALTFTEMEYQLGGLAYARRNSGC |
| | | (SEQ ID NO: 30) |
| Tryptamine | Q338X7 | MAAVTVEITRSEVLRPSPASAGGGEMVPLTVFDRAATDGYIP |
| hydroxycin- | | TMFAWDAAAAAALSNDAIKDGLAAVLSRFPHLAGRFAVDER |
| namoyltrans- | | GRKCFRLNNAGARVLEASAAGDLADALAHDVAAHVNQLYP |
| ferase/O. | | QADKDRVDEPLLQVQLTRYTCGGLVIGAVSHHQVADGQSMS |
| sativa | | VFFTEWAAAVRTAGAALPTPFLDRSAVAAPRIPPAPAFDHRN |
| | | VEFRGEGSRSHSYGALPLERMRNLAVHFPPEFVAGLKARVGG |
| | | ARCSTFQCLLAHAWKKITAARDLSPKEYTQVRVAVNCRGRA |
| | | GPAVPTDYFGNMVLWAFPRMQVRDLLSASYAAVVGVIRDA |
| | | VARVDERYIQSFVDFGEVAAGDELAPTAAEPGTAFCPDLEVD |
| | | SWIGFRFHDLDFGGGPPCAFLPPDVPIDGLLIFVPSCAAKGGVE |
| | | MFMALDDQHVEALRQICYSMD (SEQ ID NO: 31) |
| Tryptamine 4- | P0DPA7.1 | MIAVLFSFVIAGCIYYIVSRRVRRSRLPPGPPGIPIPFIGNMFDM |
| hydroxylase/ | | PEESPWLTFLQWGRDYNTDILYVDAGGTEMVILNTLETITDLL |
| P. cubensis | | EKRGSIYSGRLESTMVNELMGWEFDLGFITYGDRWREERRMF |
| | | AKEFSEKGIKQFRHAQVKAAHQLVQQLTKTPDRWAQHIRHQI |
| | | AAMSLDIGYGIDLAEDDPWLEATHLANEGLAIASVPGKFWVD |
| | | SFPSLKYLPAWFPGAVFKRKAKVWREAADHMVDMPYETMR |
| | | KLAPQGLTRPSYASARLQAMDLNGDLEHQEHVIKNTAAEVN |
| | | VGGGDTTVSAMSAFILAMVKYPEVQRKVQAELDALTNNGQI |
| | | PDYDEEDDSLPYLTACIKELFRWNQIAPLAIPHKLMKDDVYR |
| | | GYLIPKNTLVFANTWAVLNDPEVYPDPSVFRPERYLGPDGKP |
| | | DNTVRDPRKAAFGYGRRNCPGIHLAQSTVWIAGATLLSAFNI |
| | | ERPVDQNGKPIDIPADFTTGFFRHPVPFQCRFVPRTEQVSQSVS |
| | | GP (SEQ ID NO: 32) |
| Tryptamine 4- | ASU62250.1 | MIVLLVSLVLAGCIYYANARRVRRSRLPPGPPGIPLPFIGNMFD |
| hydroxylase/ | | MPSESPWLRFLQWGRDYHTDILYLNAGGTEIIILNTLDAITDLL |
| P. cyanescens | | EKRGSMYSGRLESTMVNELMGWEFDLGFITYGERWREERRM |
| | | FAKEFSEKNIRQFRHAQIKAANQLVRQLIKTPDRWSQHIRHQI |
| | | AAMSLDIGYGIDLAEDDPWIAATQLANEGLAEASVPGSFWVD |
| | | SFPALKYLPSWLPGAGFKRKAKVWKEGADHMVNMPYETMK |
| | | KLTVQGLARPSYASARLQAMDPDGDLEHQEHVIRNTATEVN |
| | | VGGGDTTVSAVSAFILAMVKYPEVQRQVQAELDALTSKGVV |
| | | PNYDEEDDSLPYLTACVKEIFRWNQIAPLAIPHRLIKDDVYRG |
| | | YLIPKNALVYANSWAVLNDPEEYPNPSEFRPERYLSSDGKPDP |
| | | TVRDPRKAAFGYGRRNCPGIHLAQSTVWIAGATLLSVFNIERP |
| | | VDGNGKPIDIPATFTTGFFRHPEPFQCRFVPRTQEILKSVSG |
| | | (SEQ ID NO: 33) |
| Tryptamine 4- | PPQ98746.1 | MINLPLSLVLVGCVYYIVSRRIRRSRLPPGPPGIPIPFVGNMYD |
| hydroxylase/ | | MPSESPWLTFLQWGREYNDRGLTTIFRVESTMVNKLMGWEF |
| P. cyanescens | | DLGFITYGDRWREERRMFSKEFSEKAIKQFRHSQVKAAHRFV |
| | | QQLAANGEPSRLPHYIRHQIAAMSLDIGYGVDLAQDDPWLEA |
| | | AHLANEGLATASVPGTFWIDSFPALKYLPSWFPGAGFKRQAK |
| | | IWKEAADHMVNMPYERMKKLAPQGLARPSYASARLQAMDP |
| | | NGDLEYQEQVIKNTASQVNVGGGDTTVSAVSAFILAMVIYPE |
| | | VQRKVQAELDAVLSNGRIPDYDEENDSMPYLTACVKELFRW |
| | | NQIAPLAIPHKLVKDDIYRGYLIPKNTLVFANSWAVLNDPEVY |
| | | PDPSVFRPERYLGPDGKPNDTVRDPRKAAFGYGRRNCPGIHL |
| | | ALSTVWITAATLLSVFDIERPVDHKGNPIDIPAAFTKGFFRHPE |
| | | PFQCRFVPRNEDSLKSLSGL (SEQ ID NO: 34) |
| Tryptamine 4- | PPQ70878.1 | MQGNPAVLLLLLTLTLCVYYAHSRRARRARLPPGPPGIPLPFV |
| hydroxylase/ | | GNLFDMPSNSPWLTYLQWGETYQTDIIYLNAGGTEMVILNTL |
| G. dilepis | | EAITDLLEKRGSIYSGRFESTMVNELMGWDFDLGFITYGERW |
| | | REERRMFSKEFNEKNIKQFRHAQIRAANLLVGQLTKTPERWH |
| | | QLIRHQIAAMSLDIGYGIDLLEGDPWLEATQLANEGLAIASVP |
| | | GSFWVDSLPILKYMPSWFPGAEFKRKAKVWRESTDHMINMP |
| | | YEKMKKLMVQDLVRPSYASARLQEMDPNGDLQHQEHVIRN |
| | | TAMEVNVGGADTTVSAVAAFILAMVKYPDVQRKVQAELDA |
| | | VGCRDELPEFDEDNDALPYLTACVKEIFRWNQVAPLAIPHRL |
| | | DKDDHYRGYIIPKNALVFANTWAVLNDPSVYPDPSEFRPERY |
| | | LGPDGKPDPRIRDPRKAAFGYGRRACPGIHLAQSTVWIVGAT |
| | | LLSVFDIERPMDANGKPIDIPAAFTTGFFRYSIHDCLVVETMHP |
| | | ANTVCVDIPNPSDADSFLVPKRLSNPHPIIDLPSRNPACQEDGV |
| | | VALSNAWRSTLPVQDV (SEQ ID NO: 35) |
| P450 | NP_01190 | MPFGIDNTDFTVLAGLVLAVLLYVKRNSIKELLMSDDGDITA |
| reductase/S. | 8.1 | VSSGNRDIAQVVTENNKNYLVLYASQTGTAEDYAKKFSKEL |
| cerevisiae | | VAKFNLNVMCADVENYDFESLNDVPVIVSIFISTYGEGDFPDG |
| | | AVNFEDFICNAEAGALSNLRYNMFGLGNSTYEFFNGAAKKAE |
| | | KHLSAAGAIRLGKLGEADDGAGTTDEDYMAWKDSILEVLKD |
| | | ELHLDEQEAKFTSQFQYTVLNEITDSMSLGEPSAHYLPSHQLN |
| | | RNADGIQLGPFDLSQPYIAPIVKSRELFSSNDRNCIHSEFDLSGS |
| | | NIKYSTGDHLAVWPSNPLEKVEQFLSIFNLDPETIFDLKPLDPT |
| | | VKVPFPTPTTIGAAIKHYLEITGPVSRQLFSSLIQFAPNADVKE |
| | | KLTLLSKDKDQFAVEITSKYFNIADALKYLSDGAKWDTVPMQ |
| | | FLVESVPQMTPRYYSISSSSLSEKQTVHVTSIVENFPNPELPDA |
| | | PPVVGVTTNLLRNIQLAQNNVNIAETNLPVHYDLNGPRKLFA |
| | | NYKLPVHVRRSNFRLPSNPSTPVIMIGPGTGVAPFRGFIRERVA |
| | | FLESQKKGGNNVSLGKHILFYGSRNTDDFLYQDEWPEYAKKL |
| | | DGSFEMVVAHSRLPNTKKVYVQDKLKDYEDQVFEMINNGAF |
| | | IYVCGDAKGMAKGVSTALVGILSRGKSITTDEATELIKMLKTS |
| | | GRYQEDVW (SEQ ID NO: 36) |
| P450 | GAQ41948.1 | MAQLDTLDLVVLAVLLVGSVAYFTKGTYWAVAKDPYASTG |
| reductase/A. | | PAMNGAAKAGKTRNIIEKMEETGKNCVIFYGSQTGTAEDYAS |
| niger | | RLAKEGSQRFGLKTMVADLEEYDYENLDQFPEDKVAVFVLA |
| | | TYGEGEPTDNAVEFYQFFTGDDVAFESGASADEKPLSKLKYV |
| | | AFGLGNNTYEHYNAMVRQVDAAFQKLGAQRIGSAGEGDDG |
| | | AGTMEEDFLAWKEPMWAALSESMDLQEREAVYEPVFCVTE |
| | | NESLSPEDESVYLGEPTQSHLQGTPKGPYSAHNPFIAPIAESRE |
| | | LFTVKDRNCLHMEISIAGSNLSYQTGDHIAVWPTNAGAEVDR |
| | | FLQVFGLEGKRDSVINIKGIDVTAKVPIPTPTTYDAAVRYYME |
| | | VCAPVSRQFVATLAAFAPDEESKAEIVRLGSDKDYFHEKVTN |
| | | QCFNIAQALQSITSKPFSAVPFSLLIEGITKLQPRYYSISSSSLVQ |
| | | KDKISITAVVESVRLPGASHMVKGVTTNYLLALKQKQNGDPS |
| | | PDPHGLTYSITGPRNKYDGIHVPVHVRHSNFKLPSDPSRPIIMV |
| | | GPGTGVAPFRGFIQERAALAAKGEKVGPTVLFFGCRKSDEDF |
| | | LYKDEWKTYQDQLGDNLKIITAFSREGPQKVYVQHRLREHSE |
| | | LVSDLLKQKATFYVCGDAANMAREVNLVLGQIIAAQRGLPA |
| | | EKGEEMVKHMRSSGSYQEDVWS (SEQ ID NO: 37) |
| P450 | PPQ81263.1 | MTDPNRTTFSSALHPLAVVSMASSSSDVFVLGLGVVLAALYIF |
| reductase/P. | | RDQLFAASKPKVAPVSTTKPANGSANPRDFIAKMKQGKKRIV |
| cyanescens | | IFYGSQTGTAEEYAIRLAKEAKQKFGLASLVCDPEEYDFEKLD |
| | | QLPEDSIAFFVVATYGEGEPTDNAVQLLQNLQDDSFEFSNGER |
| | | KLSGLKYVVFGLGNKTYEHYNLIGRTVDAQLAKMGAVRVGE |
| | | RGEGDDDKSMEEDYLEWKDGMWDAFAAAMGVEEGQGGDS |
| | | ADFVVSELESHPPEKVYLGEYSARALTKTKGIHDAKNPLAAPI |
| | | TVARELFQSVVDRNCVHVEFNIEGSGITYQHGDHVGLWPLNP |
| | | DVEVERLLCVLGLTEKRDAVISIESLDPALAKVPFPVPTTYAA |
| | | VLRHYIDVSAVAGRQILGTLSKFAPTPEAEAFLKNLNTNKEEY |
| | | HNVVANGCLKLGEILQVATGNDITVAPTPGNTTKWPIPFDIIV |
| | | SAIPRLQPRYYSISSSPKVHPNTIHATVVVLKYENVPTDPIPRK |
| | | WVYGVGSNFLLNLKHAINKEPVPFITQNGEQRVGVPEYLIAG |
| | | PRGSYKTESHFKAPIHVRRSTFRLPTNPKSPVIMIGPGTGVAPF |
| | | RGFVQERVALARRSVEKNGPESLNDWGRISLFYGCRRSDEDF |
| | | LYKDEWPQYQEELKGKFKLHCAFSRENYKPDGSKIYVQDLI |
| | | WEDREHIADAILNGKGYVYICGEAKSMSKQVEEVLARILGEA |
| | | KGGSGAVEGVAEIKLLKERSRLMLDYELAFRKFSQLQFARVA |
| | | TFAMLRSSFSLQRLFSTSSALRNVQRPIRDHLQKQDAPWEPRV |
| | | AESAQSVSEEILKAQTPLQVPTNAKATTSDSRTDSREPLTAYD |
| | | LQLVKKRVREWSEQAMIALRNRADDFTAHTKTTFSQLGLQL |
| | | NRVTGYEEIEALKRGVVEQEERINVARQAARKAKVAYEEAV |
| | | VQRSNSQREVNDLLQRKSSWMDSDVGRFTTLVRQDHLYEQE |
| | | EARAKAAVEETEEAVDREFSKLLRTILARYHEEQVWSDKIRS |
| | | ASTYGSLAALGLNMLVFIMAIVVVEPWKRRRLAQTFERKIEE |
| | | LSEENGIKLDATMLSIAQQIEQQVNLIGSLKDDISRNAPVIPEP |
| | | AQEVRAETEIEEETSPFVSLEFLPLSRRQLEVAAVGAGAFASN |
| | | LWFGFGDDALELLMLSTRANKVPPRNLSRIHMTSFIIKAHEDR |
| | | PTNSTWKQDLECAFCRIIRGELPASKVYENDKVIAILDIMPLRK |
| | | GHTLVIPKAHISRLSELPSELASSVGEAVCKVAHALTQALDNT |
| | | GLNVVCNQEYAQAVPHVHYHVIPAPKFGYPGHGVESTNGVV |
| | | GGKAPLTHREMHQKEFEAREELDDDDAKVLLKSIRARL |
| | | (SEQ ID NO: 38) |
| P450 | PPQ83917.1 | MSVSEDDHRGLLIVYATETGNAQDAADYIARQCRRIAFQCRV |
| reductase/P. | | VNIDSFLLPDLLSETIVIFVVSTTGSGVEPRSMTPLWTSLLRGD |
| cyanescens | | LPTDTFEDLYFSVFGLGDTAYEKFCWAAKKLSRRLESIGGIEF |
| | | YMRGEGDEQHPLGIDGALQPWTDGLINKLLEVAPLPPGEEIKP |
| | | INDVPLPRVLLKDTSKTALNHSADPLKSDLQYHKAIVKKNDRI |
| | | TAADWYQDVRHLVFDFQDNIQYSPGDVAVIHPVALEHDVDA |
| | | FLVTMSWQNIADEPFEIEQAMYDQSLPDHLPPITTLRTLFTRFL |
| | | DFNAVPRRSFFQYLRYFTSDEREQEKLDEFLSAAGADELYEY |
| | | CYRVRRTIHEVLSEFRHVKIPKGYIFDVFPPLRPREFSIASSIKT |
| | | HLHQIHLCVAIVKYRTKLKIPRKGVCTYYLSILKPGDTLLVGIR |
| | | RGLLRLPGKNDTPVIFIGPGTGIAPMRSAIEQRIANGCHENTLY |
| | | FGCRSASKDQHYGSEWQAYAANQELKYRSAFSRDGVEGEAR |
| | | VYVQDLIRQDSERIWDLVGHHKAWVLVSGSSNKMPAAVKD |
| | | AVAYAVEKYGGLSAEEAKEYVHLMVKEGRLIEECWS |
| | | (SEQ ID NO: 39) |
| P450 | PPQ77370.1 | MSLNGSGLLTPSSEVTLSSPSTPVLIYTFPQSNGTRPKSPVYIHI |
| reductase/P. | | DDPGVQVSTLVEYISSQPENSSSVYIYDVAEQVGFGTSTKQW |
| cyanescens | | AKQGLDISPVVDLQTRAGAGLSLVGRLSQGTSIDAVKGTVLT |
| | | AYTTPSGLALMAPSFAYLPVPSSTTRLIIQVPTVTPVGETLTLS |
| | | PTLSPLASVWSILPENVAVLLSSSPQQTVDFATLAYKVIDSHIV |
| | | HLFDHHSSAREIGRTFTPLTTIGKSGLTLQEAVKQAGYEPLEY |
| | | HGDPEAKTIVVLLNSSLALSLKAAVSVGTSGLGVVVVNVLRP |
| | | WDEAAIQTIIPSSATIVHVLDDVPNAVTQGSLYVDVFSALWST |
| | | TPKRSVHSHRITPSQTQKFIAAGGEFLRFVEEVTHIAVSEPSVA |
| | | SIKKTLFFSVPDSPLALLSRFVQELFLTKRTISSRHLTDYDVYS |
| | | KPGGISAQRLLISRDKSTDNVPVQAILPLDPNSVGHSDFLGVL |
| | | DHNLLKTHSLLKHAKKGSIVVVASPWTPDEFSANITYEVAEVI |
| | | TSRQLSVYTIDVKSIANDLELFIQEQKIEKGEAQVLLFEFVFLR |
| | | FYLGAAATEQAIIQLMSVLFDDIDLTKFSAAAWLGLKPVVVA |
| | | LPEVTPSDSPTLKEFEANAIAVETSEGQTVVNGARLSTWHDA |
| | | AKHLLFPSAFSPPTDPDSLSNPALRPEVPDTTFLVTCTVNKRLT |
| | | PLEYDRNVFHLEFDTSGTGLKYAIGEALGVHGWNDEQEVLDF |
| | | CEWYGVDPDRLITIPVIGSDDGKMHTRTVLQALQQQIDLFGRP |
| | | PKSFYTDLAEYATVDVDRYALRFIGSPEGVSTFKKMSEKDTV |
| | | SFGDVLKKYKSARPGIERLCELIGDIKPRHYSIASAQSVVGDR |
| | | VDLLVVTVDWLTPEGSPRYGQCTRYLAGLKIGQKVTVSIKPS |
| | | VMKLPPNLKQPLIMAGLGTGAAPFRAFLQHLAWLASKGEEIG |
| | | PVFYYFGSRYQAAEYLYGEEIEAFILGGVITRAGLAFSRDGPK |
| | | KVYIQHKMLEDSETLAKMLHDDDGVFYLCGPTWPVPDVYEA |
| | | LVNALVKYKGSDPVKAGEYLESLKEEERYVLEVY |
| | | (SEQ ID NO: 40) |
| 4- | P0DPA8 | MAFDLKTEDGLITYLTKHLSLDVDTSGVKRLSGGFVNVTWRI |
| hydroxy- | | KLNAPYQGHTSIILKHAQPHMSTDEDFKIGVERSVYEYQAIKL |
| tryptamine | | MMANREVLGGVDGIVSVPEGLNYDLENNALIMQDVGKMKT |
| kinase/ | | LLDYVTAKPPLATDIARLVGTEIGGFVARLHNIGRERRDDPEF |
| P. cubensis | | KFFSGNIVGRTTSDQLYQTIIPNAAKYGVDDPLLPTVVKDLVD |
| | | DVMHSEETLVMADLWSGNILLQLEEGNPSKLQKIYILDWELC |
| | | KYGPASLDLGYFLGDCYLISRFQDEQVGTTMRQAYLQSYART |
| | | SKHSINYAKVTAGIAAHIVMWTDFMQWGSEEERINFVKKGV |
| | | AAFHDARGNNDNGEITSTLLKESSTA |
| | | (SEQ ID NO: 41) |
| 4- | PPQ83229.1 | MAFDLKTPEGLLLYLTRHLSLDVDPSGVKRLSGGFVNVTWRI |
| hydroxy- | | RLNAPYQGHTSIILKHAQPHLSSDEDFKIGVERSAYEYQALKV |
| tryptamine | | MSANQEVLGGDDSRVSVPEGLHYDVENNALIMQDVGTMKT |
| kinase/P. | | LLDYATAKPPLSTEIASLVGTEIGAFIARLHNLGRKRRDQPAF |
| cyanescens | | KFFSGNIVGRTTADQLYQTIIPNAAKYGINDPLLPTVVKDLVG |
| | | EVMNSEETLIMADLWSGNILLEFVEGNPSELKKIWLVDWELC |
| | | KYGPASLDMGYFLGDCYLIARFQDELVGTTMRKAYLKGYAR |
| | | TAKGTINYSKVTASIGAHLVMWTDFMKWGNYEEREEFVKKG |
| | | VEALHDAWEDNNDGEITSVLVNEASST |
| | | (SEQ ID NO: 42) |
| 4- | PPQ98758.1 | MAFDLKTVEGLIVYLTKCLSLEVDSSGVKRLSGGFVNVTWRI |
| hydroxy- | | RLNAPYQGHTSIILKHAQPHMSTDKDFKIGVERSVYEYQALK |
| tryptamine | | VISANREALGGIDSRVSAPEGLHYDVENNALIMQDVGTLKTL |
| kinase/P. | | MDYVIEKPAISTEMARLIGTEIGDFVARLHSIGRQKRDQPDFK |
| cyanescens | | FFSGNIVGRTTADQLYQTILPNTAKYGIDDPLLPTVVKDLVDE |
| | | AMQSEETLIMADLWTGNILVEFEEGNLSVLKKIWLVDWELCK |
| | | YGPVRLDMGYFLGDCFLISRFKNEQVAKAMRQAFLQRYNRV |
| | | SDTPINYSVATTGIAAHIVMWTDFMNWGTEEERKEYVKKGV |
| | | AGIHDGRNHNVDGEITSILMQEASTA |
| | | (SEQ ID NO: 43) |
| 4- | PPQ70874.1 | MTFDLKTEEGLLVYLTQHLSLDVDLDGLKRLSGGFVNITWRI |
| hydroxy- | | RLNAPFKGYTNIILKHAQPHLSSDENFKIGVERSAYEYRALKI |
| tryptamine | | VSESPILSGDDNLVFVPQSLHYDVVHNALIVQDVGSLKTLMD |
| kinase/ | | YVTARPSLSSEMAKLVGGQIGAFIARLHNIGRENKDHPEFNFF |
| G. dilepis | | SGNIVGRTTAVQLYETIVPNATKYDIDDPIIPVVVQELIEEVKG |
| | | SDETLIMADLWGGNILLEFGKDSSDLGKIWVVDWELCKYGPP |
| | | SLDMGYFLGDCFLLAQFQDEKVATAMRRAYLENYAKIAKVP |
| | | MDYDRSTTGIGAHLVMWTDFMNWGSDEERKTSVEKGVRAF |
| | | HDAKRDNKEGEIPSILLRESSRT (SEQ ID NO: 44) |
| Multicopper | NP 11661 | MLFYSFVWSVLAASVALAKTHKLNYTASWVTANPDGLHEK |
| oxidase/S. | 2.1 | RMIGFNGEWPLPDIHVEKGDRVELYLTNGFQDNTATSLHFHG |
| cerevisiae | | LFQNTSLGNQLQMDGPSMVTQCPIVPGQTYLYNFTVPEQVGT |
| | | FWYHAHMGAQYGDGMRGAFIIHDPEEPFEYDHERVITLSDHY |
| | | HENYKTVTKEFLSRYNPTGAEPIPQNILFNNTMNVTLDFTPGE |
| | | TYLFRFLNVGLFVSQYIILEDHEMSIVEVDGVYVKPNFTDSIYL |
| | | SAGQRMSVLIKAKDKMPTRNYAMMQIMDETMLDVVPPELQ |
| | | LNQTIQMRYGHSLPEARALNIEDCDLDRATNDFYLEPLIERDL |
| | | LAHYDHQIVMDVRMVNLGDGVKYAFFNNITYVTPKVPTLTT |
| | | LLTSGKLASDPRIYGDNINAQLLKHNDIIEVVLNNYDSGRHPF |
| | | HLHGHNFQIVQKSPGFHVDEAYDESEQDEMTVPYNESAPLQP |
| | | FPERPMVRDTVVLEPSGHVVLRFRADNPGVWYFHCHVDWHL |
| | | QQGLASVFIEAPVLLQEREKLNENYLDICKAADIPVVGNAAG |
| | | HSNDWFDLKGLPRQPEPLPKGFTTEGYLALIISTIIGVWGLYSI |
| | | AQYGIGEVIPNDEKVYHTLREILAENEIEVSRG* |
| | | (SEQ ID NO: 45) |
| aralkylamine | O02785 | MSTPSIHCLKPSPLHLPSGIPGSPGRQRRHTLPANEFRCLTPKD |
| N- | | AAGVFEIEREAFISVSGNCPLNLDEVRHFLTLCPELSLGWFVE |
| acetyltrans- | | GRLVAFIIGSLWDEERLTQESLTLHRPGGRTAHLHALAVHHSF |
| ferase/Bos | | RQQGKGSVLLWRYLQHAGGQPAVRRAVLMCEDALVPFYQR |
| taurus | | FGFHPAGPCAVVVGSLTFTEMHCSLRGHAALRRNSDR |
| | | (SEQ ID NO: 46) |
| Tryptamine 5- | O96370 | MISTESDLRRQLDENVRSEADESTKEECPYINAVQSHHQNVQ |
| hydroxylase/ | | EMSIIISLVKNMNDMKSIISIFTDRNINILHIESRLGRLNMKKHT |
| Schistoso- | | EKSEFEPLELLVHVEVPCIEVERLLEELKSFSSYRIVQNPLMNL |
| mamansoni | | PEAKNPTLDDKVPWFPRHISDLDKVSNSVLMYGKELDADHP |
| | | GFKDKEYRKRRMMFADIALNYKWGQQIPIVEYTEIEKTTWGR |
| | | IYRELTRLYKTSACHEFQKNLGLLQDKAGYNEFDLPQLQVVS |
| | | DFLKARTGFCLRPVAGYLSARDFLSGLAFRVFYCTQYIRHQA |
| | | DPFYTPEPDCCHELLGHVPMLADPKFARFSQEIGLASLGTSDE |
| | | EIKKLATCYFFTIEFGLCRQDNQLKAYGAGLLSSVAELQHALS |
| | | DKAVIKPFIPMKVINEECLVTTFQNGYFETSSFEDATRQMREF |
| | | VRTIKRPFDVHYNPYTQSIEIIKTPKSVAKLVQDLQFELTAINE |
| | | SLLKMNKEIRSQQFTTNKIVTENRSSGS* |
| | | (SEQ ID NO: 47) |
| Acetylsero- | P46597 | MGSSEDQAYRLLNDYANGFMVSQVLFAACELGVFDLLAEAP |
| tonin O- | | GPLDVAAVAAGVRASAHGTELLLDICVSLKLLKVETRGGKAF |
| methyltrans- | | YRNTELSSDYLTTVSPTSQCSMLKYMGRTSYRCWGHLADAV |
| ferase/Homo | | REGRNQYLETFGVPAEELFTAIYRSEGERLQFMQALQEVWSV |
| sapiens | | NGRSVLTAFDLSVFPLMCDLGGGAGALAKECMSLYPGCKITV |
| | | FDIPEVVWTAKQHFSFQEEEQIDFQEGDFFKDPLPEADLYILA |
| | | RVLHDWADGKCSHLLERIYHTCKPGGGILVIESLLDEDRRGPL |
| | | LTQLYSLNMLVQTEGQERTPTHYHMLLSSAGFRDFQFKKTGA |
| | | IYDAILARK (SEQ ID NO: 48) |
| Noribogaine | A0A2Z5P0 | DAMKSAELFKAQAHIFKQVFCFTNGASLKCAVQLGIPDAIDN |
| 10-O- | W7 | HGKAMTLSELTDALPINPSKAPHIHRLMRILVTAGFFVEERLG |
| methyltrans- | | NGKEEKANGYALTPSSRLLLKNKPLSLRASALTMLDPVTVKT |
| ferase/ | | WNALSEWFQNEDQTAFETAHGKNMWDFFAEDPGLSKKFNES |
| Tabernan- | | MASDSQLVTEVLVTKCKFVFEGLTSMVDVGGGTGTVAGAIA |
| theiboga | | KTFPSLRCTVFDLPHVVANLEPTENLDFVAGDMFGKIPPANAI |
| | | FLKWVLHDWNDEDCVKILKNCKRAIPGKEKGGKVIIVDIIME |
| | | TEKHDIDEFDYAKMCMDMEMLVLCNSKERTEKELAMLVSEA |
| | | GFSGYKIFPVLGIRSLIEVYP (SEQ ID NO: 49) |

### EXAMPLES

These examples are provided for illustrative purposes only and not to limit the scope of the claims provided herein.

### Example 1: Detection of 4-hydroxytryptamine in Engineered Cells

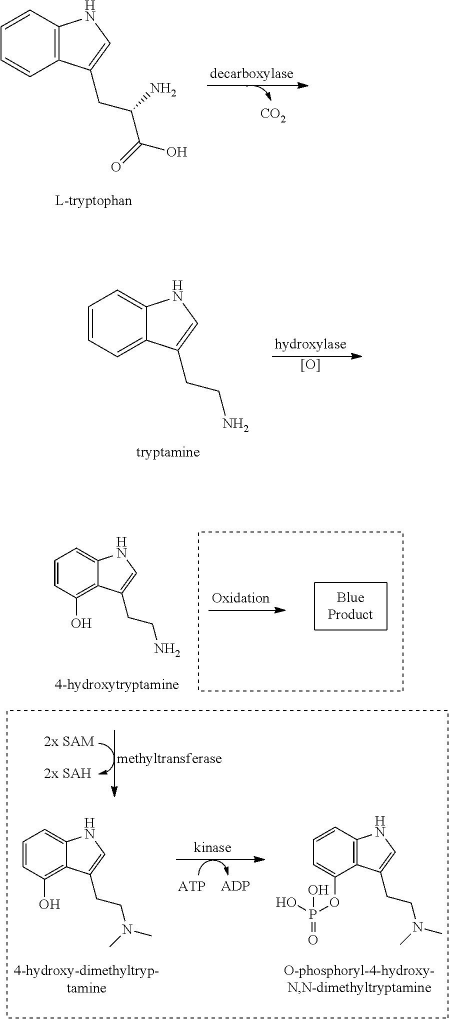

4-hydroxytryptamine can be oxidized to a blue product to screen for high biosynthetic flux through the upstream pathway (blue box). Alternatively, 4-hydroxytryptamine can be employed for conversion to O-phosphoryl-4-hydroxy-N,N-dimethyltryptamine, or psilocybin, by expression of the methyltransferase or kinase.

### Example 2. Yeast and Bacterial Strains and Growth Conditions

Single gene expression plasmids were transformed into chemically competent TG1 E. coli and multigene plasmids were transformed into TransforMax™ EPI300™ (Epicentre) electrocompetent E. coli. Strains were constructed using chemical or electro-competency. Selections were performed on LB containing ampicillin (25 mg/L) and kanamycin (25 mg/L) as indicated. The background strain MG1655 with lambda DE3 (a phage construct that expresses T7 RNA polymerase under the control of a lacUV5 promoter) was used as a host strain and propagated at 37° C. S. cerevisiae strain BY4741 (MATa his3Δ1 leu2Δ0 met15Δ0 ura3Δ0) was used for experiments in this study and propagated at 30° C.

E. coli cultures were propagated in LB broth (1-liter medium contained 10 grams of tryptone, 5 grams of yeast extract, and 10 grams of sodium chloride). Yeast cultures were grown in YPD (10 g/L Bacto Yeast Extract; 20 g/L Bacto Peptone; 20 g/L D-glucose). Lithium acetate transformation method was used to transform yeast with plasmids containing the respective auxotrophic markers. Selection was performed on synthetic dropout media (6.7 g/L Difco yeast nitrogen base without amino acids; 2 g/L synthetic defined amino acid mix minus the respective autotrophy, without yeast nitrogen base (US Biological); 20 g/L D-glucose or the respective carbon source; 20 g/L BD Difco agar was used for plates). pH was adjusted when appropriate with NaOH or HCl.

### Plasmids and Cloning

A hierarchical Golden Gate cloning scheme was used for assembling coding sequence part plasmids, yeast protein expression cassettes and multigene plasmids. All protein coding sequences were synthesized or PCR amplified to omit internal BsaI and BsmBI sites for use in golden gate cloning. The protein coding sequences for fungal pathway enzymes were codon optimized for E. coli or S. cerevisiae and synthesized by Integrated DNA Technologies (Coralville, IA).

### Example 3. Production of Substituted Tryptamines by Fed Substituted Indoles and Anthranilates

The background strain MG1655 with lambda DE3 is a phage construct that expresses T7 RNA polymerase under the control of a lacUV5 promoter and was used as a host strain. The strain was modified to have the tryptophan biosynthetic pathway (e.g., trpE, trpD, trpC, trpB, and trpA) and tryptophan deaminase (tnaA) knocked out. This genetic material was removed by modified λ red system as described by Datsenko and Wanner (2000). The resulting strain was named bNAB001.

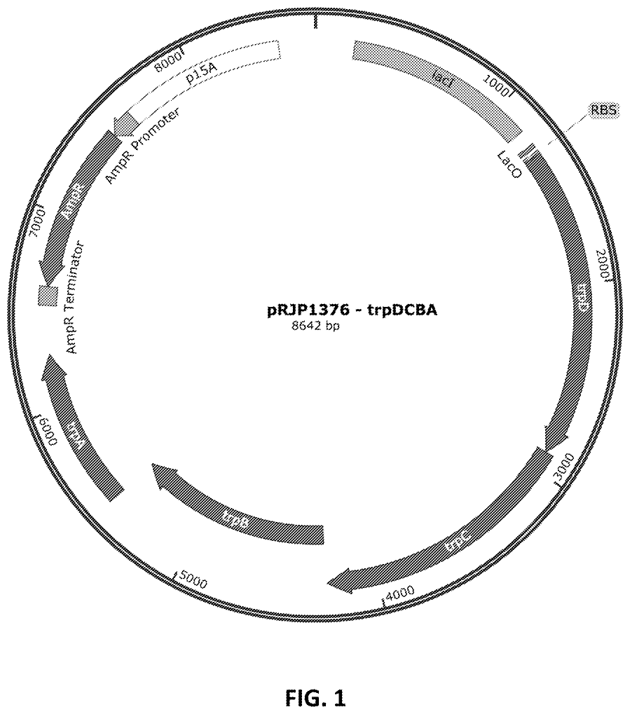

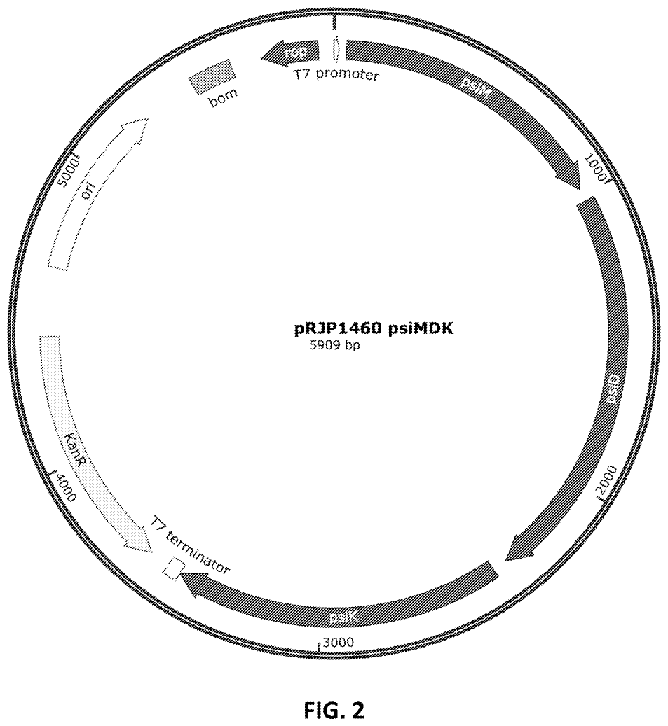

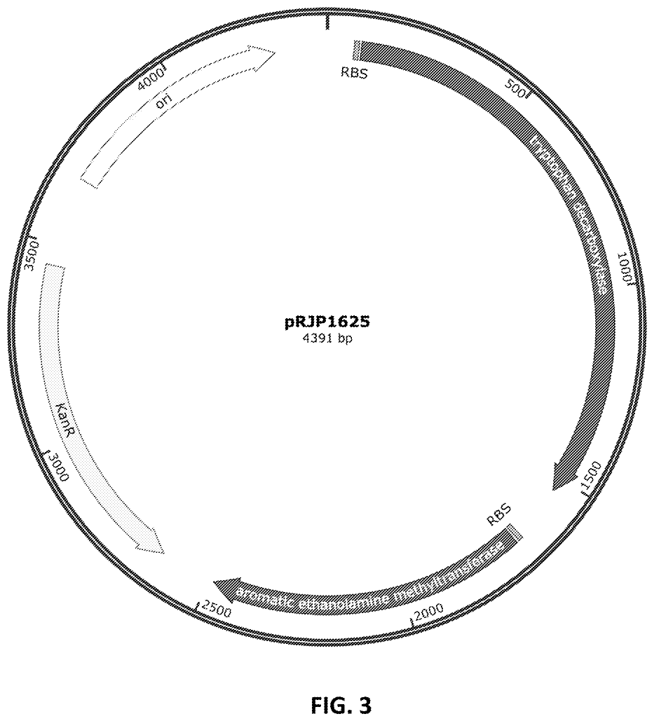

The tryptophan biosynthetic pathway, trpDCBA (SEQ ID NO: 1), was cloned under Lad operon control in an ampicillin resistance plasmid with p15a origin and was named pRJP1376 (FIG. 1). This plasmid was introduced into bNAB001 and resulted in strain bNAB002. Accordingly, the bNAB002 strain, upon isopropyl 0-D-1-thiogalactopyranoside (IPTG) induction, allows for expression of trpDCBA operon for substituted tryptophan biosynthesis upon addition of substituted anthranilate and substituted indole addition. To further convert resulting substituted tryptophan compounds into downstream tryptamines, two plasmids were cloned. Coding sequences for tryptophan decarboxylase from P. cubensis (psiD, SEQ ID NO: 14) for converting substituted tryptophans to tryptamines; 4-hydroxytryptamine kinase from P. cubensis (psiK, SEQ ID NO: 41); and tryptamine N-methyltransferase from P. cubensis (psiM, SEQ ID NO: 21) were cloned into a plasmid containing a kanamycin resistance gene and BR322 origin of replication and was named pRJP1460 (FIG. 2). Coding sequences were oriented in a multi-cistronic operon downstream of a T7 promoter sequence. This plasmid was transformed into bNAB002 and the resulting strain was named bNAB003. Coding sequences for tryptophan decarboxylase from B. atrophaeus (SEQ ID NO: 19) and aromatic ethanolamine methyltransferase from H. sapiens (SEQ ID NO: 25) were cloned downstream of promoter and ribosome binding sequences with a kanamycin resistance gene and BR322 origin of replication sequences to form plasmid pRJP1625 (FIG. 3). The pRJP1625 plasmid was transformed into bNAB002 and the resulting strain was named bNAB004.

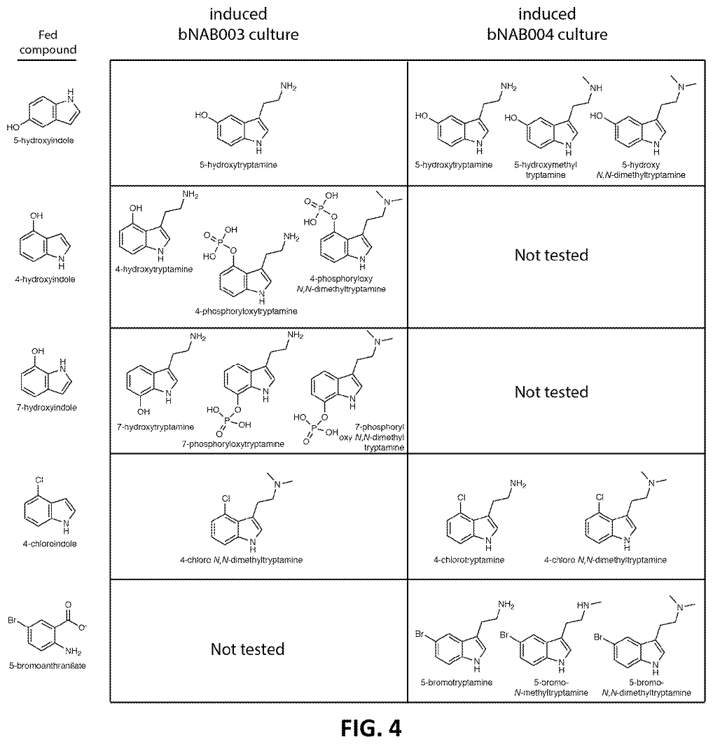

An overnight bNAB003 culture was used to inoculate 1 L of LB (plus kanamycin and ampicillin) culture at 0.1 OD₆₀₀. The culture was grown to OD₆₀₀ of 0.5 and cooled to 18° C. on ice before induction with 0.5 mM of IPTG for expression of trpDCBA pathway proteins and psiMDK pathway proteins. The culture was transferred to a shaker at 18° C. with 200 RPM shaking for 16 hours. The cells were harvested, washed with sterile water, and resuspended in M9 media (0.2% glucose, 40 mM Na₂HPO₄, 20 mM KH₂PO₄, 1 mM MgSO₄, 0.1 mM CaCl₂, 0.5 mM IPTG, with added 100 mg/L of L-serine, 2 g/L sodium citrate, and 2 g/L yeast extract). The culture was split into 10 mL cultures in sterile culture tubes. 4 mM of 5-hydroxyindole, 4-hydroxyindole, 7-hydroxyindole, and 4-chloroindole were added to separate tubes. After 3 days of incubation at 30° C., media from cultures were sampled by centrifuging 1 mL of culture at 18000 rpm and transferring clarified media to sample vials. Analysis was performed by chromatography/mass spectrometry (LCMS) with a 1260 Infinity LC System connected to a 6120 Quadrupole Mass Spectrometer (Agilent Technologies). Zorbax Eclipse Plus C18 guard column (4.6 cm×12.5 cm, 5 μm packing, Agilent Technologies) was connected to a Zorbax Eclipse Plus C18 column (4.6 mm×100 mm, 3.5 μm packing, Agilent Technologies) at 20° C. using a 0.5 mL/min. flow rate. Water and acetonitrile mobile phases contained 0.1% formic acid as the pH modifier. The elution gradient (water:acetonitrile volume ratio) was as follows: 98:2 (0-2 min), linear ramp from 98:2 to 5:95 (2-17 min), 5:95 (17-22 min), linear ramp from 5:95 to 98:2 (22-23 min), and 98:2 (23-28 min). Absorbance was measured using a diode array detector for UV—Vis analysis. MS was conducted in atmospheric pressure ionization-positive electrospray (API-ES positive) mode at 100-V fragmentor voltage with ion detection set to both full scanning mode (50-1200 m/z). Detection of tryptamines was conducted by extraction of ion masses of corresponding tryptamine not found in the unfed control sample. In the 5-hydroxyindole fed culture, 5-hydroxytryptamine was detected. In the 4-hydroxyindole culture, 4-hydroxytryptamine, 4-phosphoryloxytryptamine, and 4-phosphoryloxy-N,N-dimethyltryptamine were detected. In the 7-hydroxyindole fed culture, 7-hydroxytryptamine, 7-phosphoryloxytryptamine, and 7-phosphoryloxy-N,N-dimethyltryptamine were detected. In the 4-chloroindole fed culture, 4-chloro-N,N-dimethyltryptamine was detected (see FIG. 4).

An overnight bNAB004 culture was used to inoculate 1 L of LB (plus kanamycin and ampicillin) culture at 0.1 OD₆₀₀. The culture was grown to OD₆₀₀ of 0.5 and cooled to 18° C. on ice before induction with 0.5 mM of IPTG for expression of trpDCBA pathway proteins and psiMDK pathway proteins. The culture was transferred to a shaker at 18° C. with 200 RPM shaking for 16 hours. The cells were harvested, washed with sterile water, and resuspended in M9 media (0.2% glucose, 40 mM Na₂HPO₄, 20 mM KH₂PO₄, 1 mM MgSO₄, 0.1 mM CaCl₂, 0.5 mM IPTG, with added 100 mg/L of L-serine, 2 g/L sodium citrate, and 2 g/L yeast extract). The culture was split into 10 mL cultures in sterile culture tubes. 4 mM of 5-hydroxyindole, 4-chloroindole, and 5-bromoanthranilate were added to separate tubes. After 3 days of incubation at 30° C., media from cultures were sampled by centrifuging 1 mL of culture at 18000 rpm and transferring clarified media to sample vials. Analysis was performed by chromatography/mass spectrometry (LCMS) with a 1260 Infinity LC System connected to a 6120 Quadrupole Mass Spectrometer (Agilent Technologies). Zorbax Eclipse Plus C18 guard column (4.6 cm×12.5 cm, 5 μm packing, Agilent Technologies) was connected to a Zorbax Eclipse Plus C18 column (4.6 mm×100 mm, 3.5 μm packing, Agilent Technologies) at 20° C. using a 0.5 mL/min flow rate. Water and acetonitrile mobile phases contained 0.1% formic acid as the pH modifier. The elution gradient (water: acetonitrile volume ratio) was as follows: 98:2 (0-2 min), linear ramp from 98:2 to 5:95 (2-17 min), 5:95 (17-22 min), linear ramp from 5:95 to 98:2 (22-23 min), and 98:2 (23-28 min). Absorbance was measured using a diode array detector for UV—Vis analysis. MS was conducted in atmospheric pressure ionization-positive electrospray (API-ES positive) mode at 100-V fragmentor voltage with ion detection set to both full scanning mode (50-1200 m/z). Detection of tryptamines was conducted by extraction of ion masses of corresponding tryptamine not found in the unfed control sample. In the 5-hydroxyindole fed culture, 5-hydroxytryptamine, 5-hydroxymethyltryptamine, and 5-hydroxy-N,N-dimethyltryptamine were detected. In the 4-chloroindole fed culture, 4-chlorotryptamine and 4-chloro-N,N-dimethyltryptamine were detected. In the 5-bromoanthranilate fed culture, 5-bromotryptamine, 5-bromo-N-methyltryptamine, and 5-bromo-N,N-dimethyltryptamine were detected (see FIG. 4).

### Example 4. Production of Substituted Tryptamines by Engineered Yeast

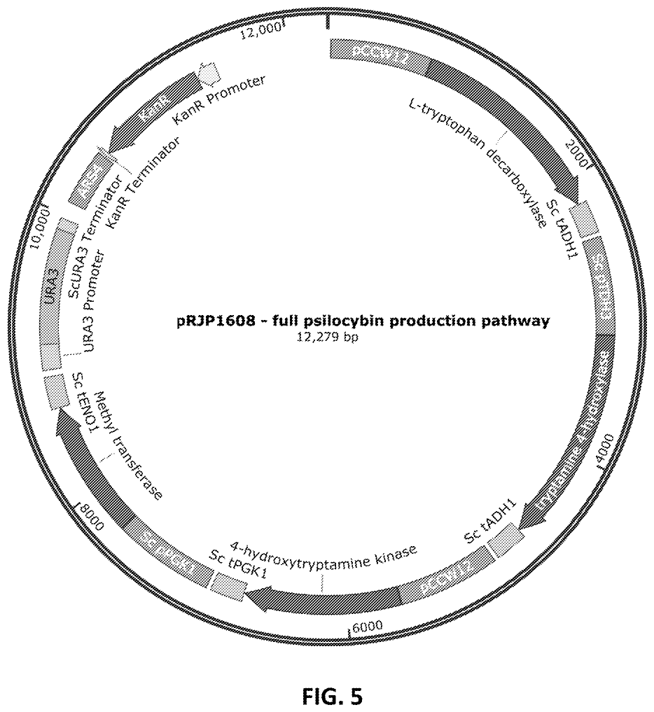

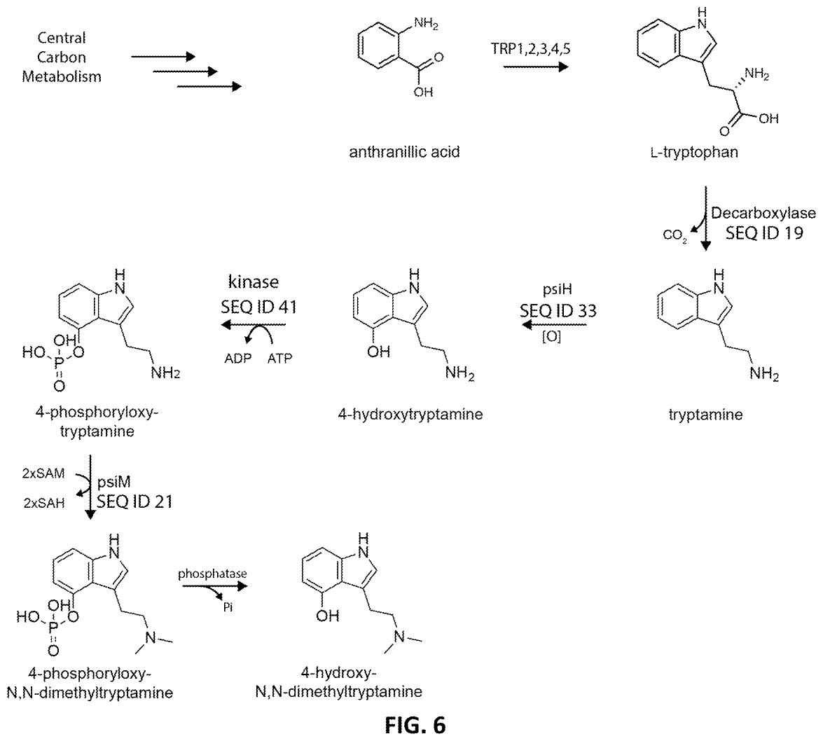

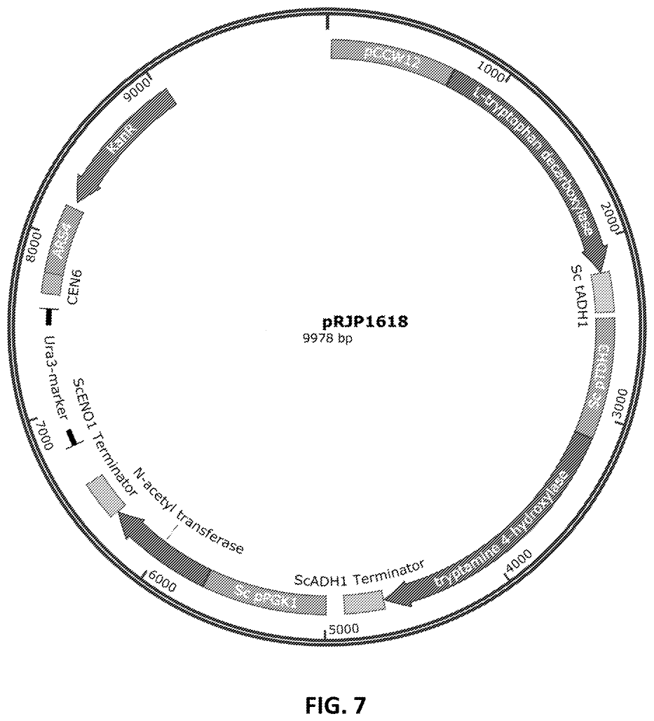

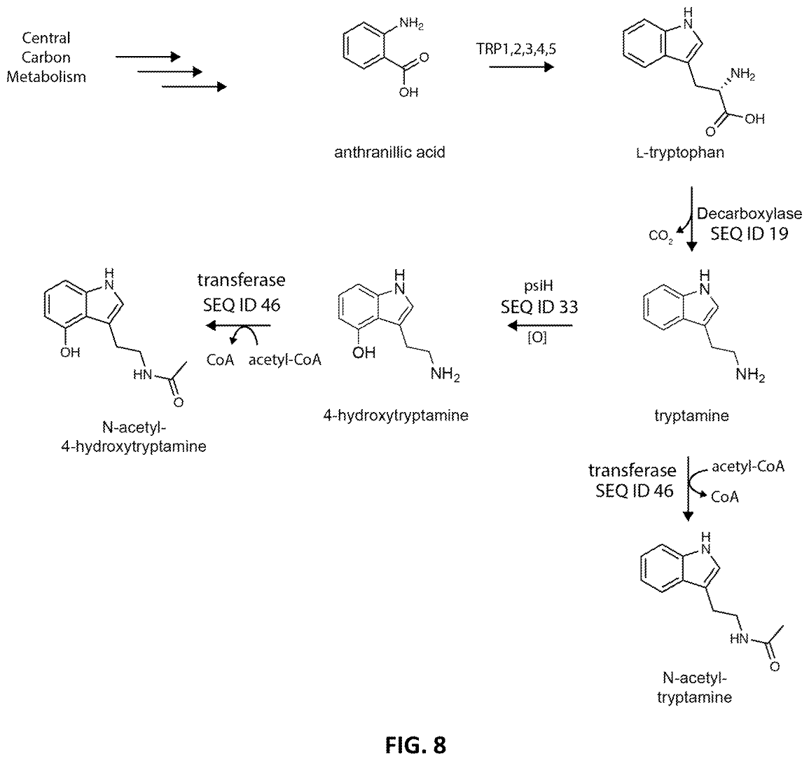

Anthranilate biosynthetically produced from central carbon metabolism (i.e., hydrogen substituted anthranilate) can be metabolized to form substituted tryptamines with genetic modification. Substitutions of the amine position of tryptamine and indole ring were investigated. A multigene plasmid with CEN6/ARS4 replication sequences, URA3 expression cassette and kanamycin resistance was cloned to contain coding sequences for tryptophan decarboxylase from B. atrophaeus (SEQ ID NO: 19), tryptophan 4-hydroxylase from P. cyanescens (SEQ ID NO: 33), 4-hydroxytryptamine kinase from P. cubensis (psiK, SEQ ID NO: 41), and tryptamine N-methyltransferase from P. cubensis (psiM, SEQ ID NO: 21) under control of high activity yeast promoter and terminator pairs (e.g., promoters pCCW12, pTDH3, and pPGK1, or terminators tADH1, tPGK1, and tENO1) and was named plasmid pRJP1608 (FIG. 5). The biosynthetic pathway converting anthranilic acid present in the yeast metabolism to tryptamines by enzymes encoded in pRJP1608 is outlined in FIG. 6. A second multigene plasmid with CEN6/ARS4 replication sequences, URA3 expression cassette, and kanamycin resistance was cloned to contain coding sequences for tryptophan decarboxylase from B. atrophaeus (SEQ ID NO: 19), tryptophan 4-hydroxylase from P. cyanescens (SEQ ID NO: 33), and aralkylamine N-acetyltransferase from B. taurus (SEQ ID NO: 46) under control of high activity yeast promoter and terminator pairs (e.g., promoters pCCW12, pTDH3, and pPGK1, or terminators tADH1, tPGK1, and tENOl) and was named plasmid pRJP1618 (FIG. 7). The biosynthetic pathway converting anthranilic acid present in the yeast metabolism to tryptamines by enzymes encoded in pRJP1618 is outlined in FIG. 8.

S. cerevisiae strain BY4741 (MATa his3Δ1 leu2Δ0 met15Δ0 ura3Δ0) was used for experiments in this study and propagated at 30° C. The pRJP1608 plasmid was transformed into BY4741 by lithium acetate protocol and selected for on synthetic complete medium lacking uracil. The resulting strain, yNAB001, was isolated and genotyped. The pRJP1618 plasmid was transformed into BY4741 by lithium acetate protocol and selected for on synthetic complete medium lacking uracil. The resulting strain, yNAB002, was isolated and genotyped.

Colonies of yNABOO1 and yNAB002 were used to inoculate 5 mL cultures of synthetic complete medium and were grown at 30° C. in a rotary shaker at 225 rpm. Media from cultures were sampled by centrifuging 1 mL of culture at 18000 rpm and transferring clarified media to sample vials. Analysis was performed by chromatography/mass spectrometry (LCMS) with a 1260 Infinity LC System connected to a 6120 Quadrupole Mass Spectrometer (Agilent Technologies). Zorbax Eclipse Plus C18 guard column (4.6 cm×12.5 cm, 5 μm packing, Agilent Technologies) was connected to a Zorbax Eclipse Plus C18 column (4.6 mm×100 mm, 3.5 μm packing, Agilent Technologies) at 20° C. using a 0.5 mL/min. flow rate. Water and acetonitrile mobile phases contained 0.1% formic acid as the pH modifier. The elution gradient (water:acetonitrile volume ratio) was as follows: 98:2 (0-2 min), linear ramp from 98:2 to 5:95 (2-17 min), 5:95 (17-22 min), linear ramp from 5:95 to 98:2 (22-23 min), and 98:2 (23-28 min). Absorbance was measured using a diode array detector for UV—Vis analysis. MS was conducted in atmospheric pressure ionization-positive electrospray (API-ES positive) mode at 100-V fragmentor voltage with ion detection set to both full scanning mode (50-1200 m/z).

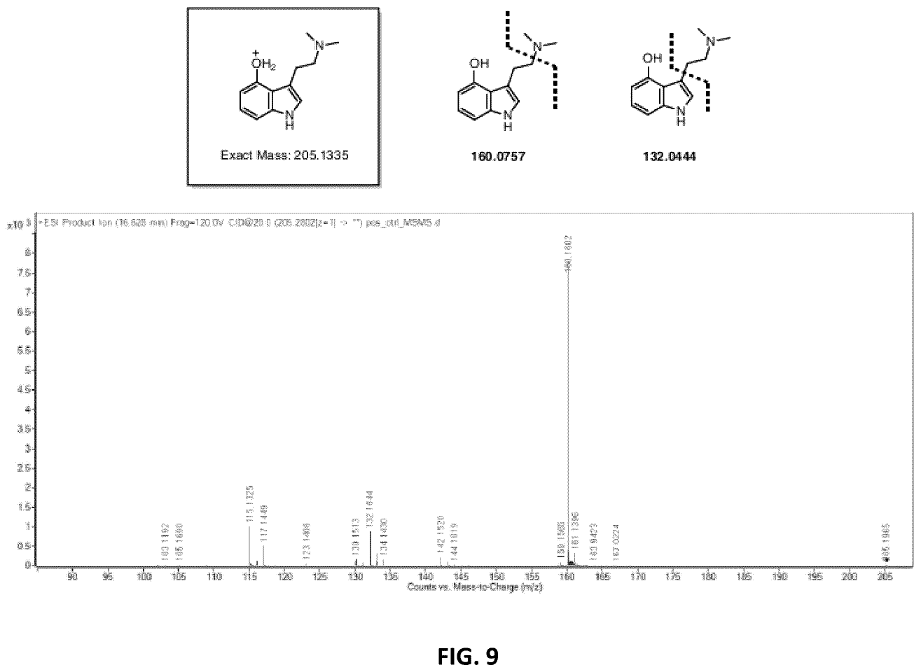

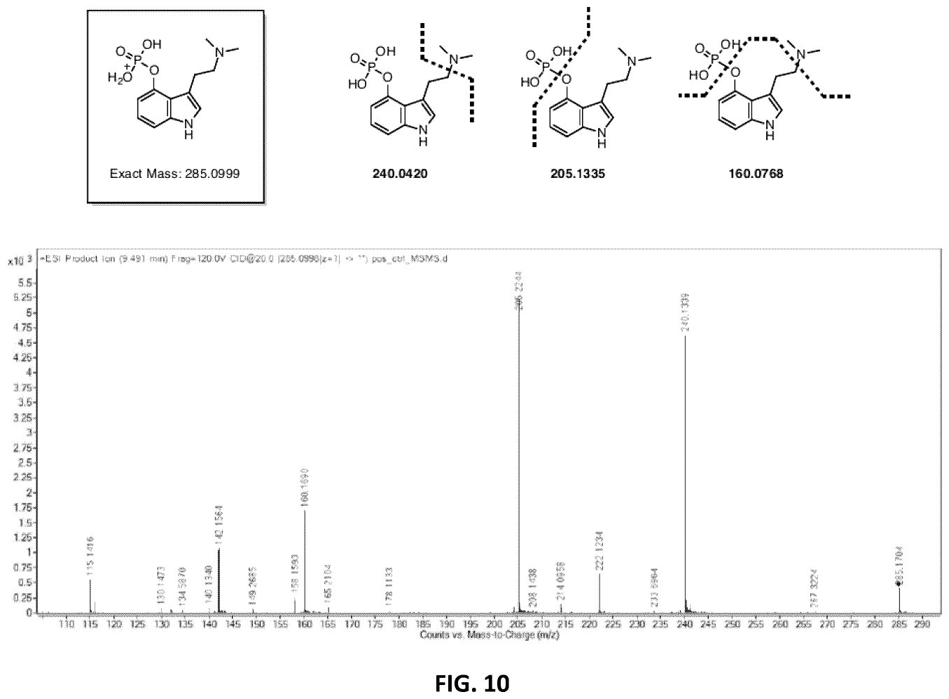

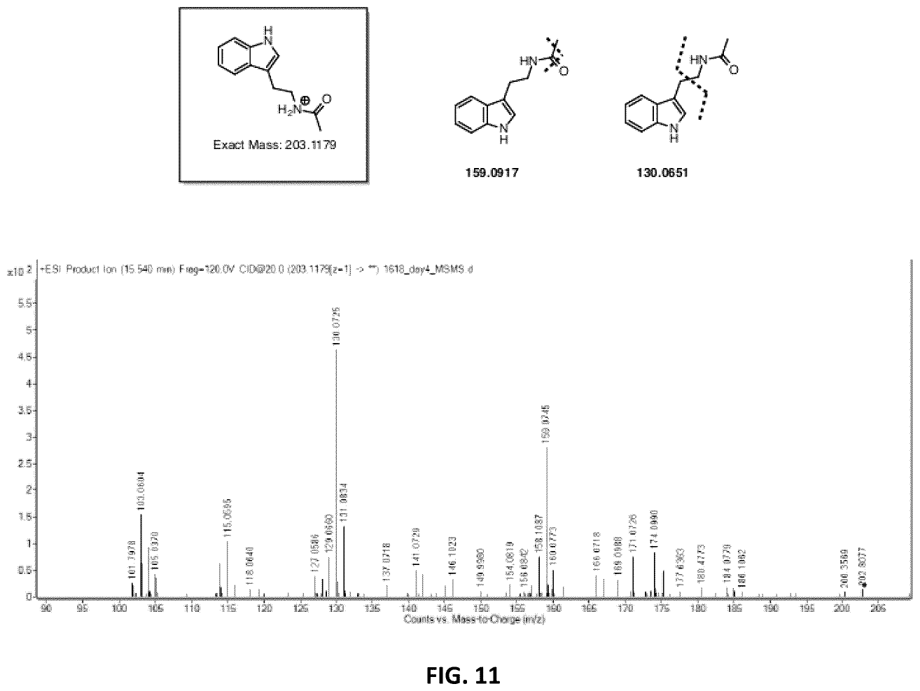

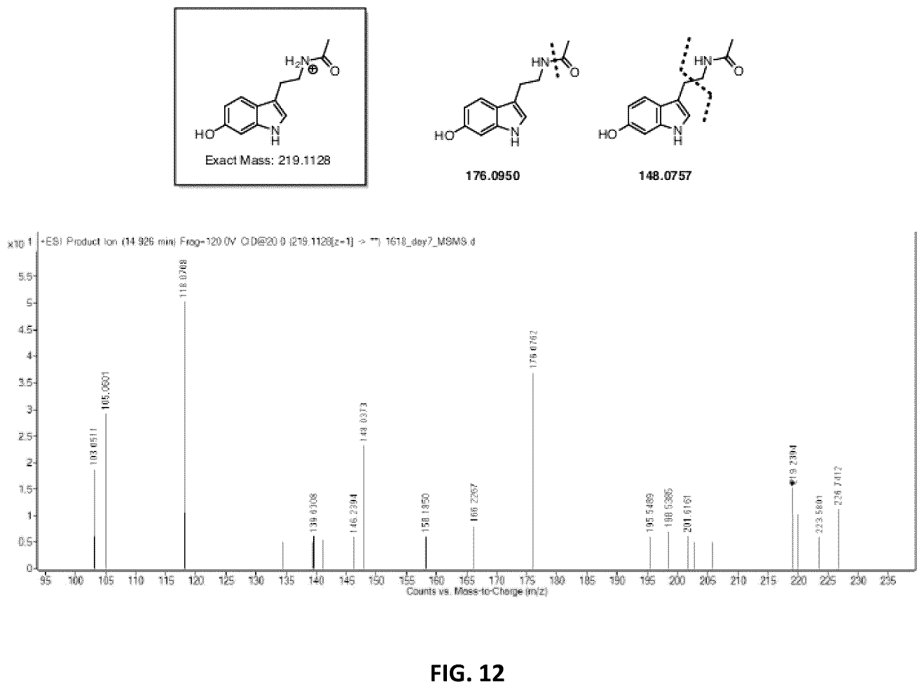

Detection of tryptamines was conducted by extraction of ion masses of corresponding tryptamine not found in the unfed control sample. Additionally, tandem MS/MS was conducted. In the culture of yNAB001, ion masses for tryptamine, 4-hydroxytryptamine, 4-phosphoryloxytryptamine, 4-hydroxy-N,N-dimethyltryptamine, and 4-phosphoryloxy-N,N-dimethyltryptamine were detected (FIG. 6). Tandem MS/MS fragmentation data was collected for 4-hydroxy-N,N-dimethyltryptamine at a positive ion mass of 205.1335 m/z (FIGS. 9) and 4-phosphoryloxy-N,N-dimethyltryptamine at a positive ion mass of 285.0999 m/z (FIG. 10). In the culture of yNAB002, ion masses for tryptamine, 4-hydroxytryptamine, N-acetyl-tryptamine, and 4-hydroxy-N-acetyl-tryptamine were detected (FIG. 8). Tandem MS/MS fragmentation data was collected for N-acetyl-tryptamine at a positive ion mass of 203.1179 m/z (FIGS. 11) and 4-hydroxy-N-acetyl-tryptamine at a positive ion mass of 219.1128 (FIG. 12). Media from an untransformed strain of BY4741 detected only trace levels of tryptamine and no other aforementioned tryptamines from yNAB001 and yNAB002 media.

### Example 5. Production of Tryptamine Derivatives from Fed Tryptamines Using Engineered Cells

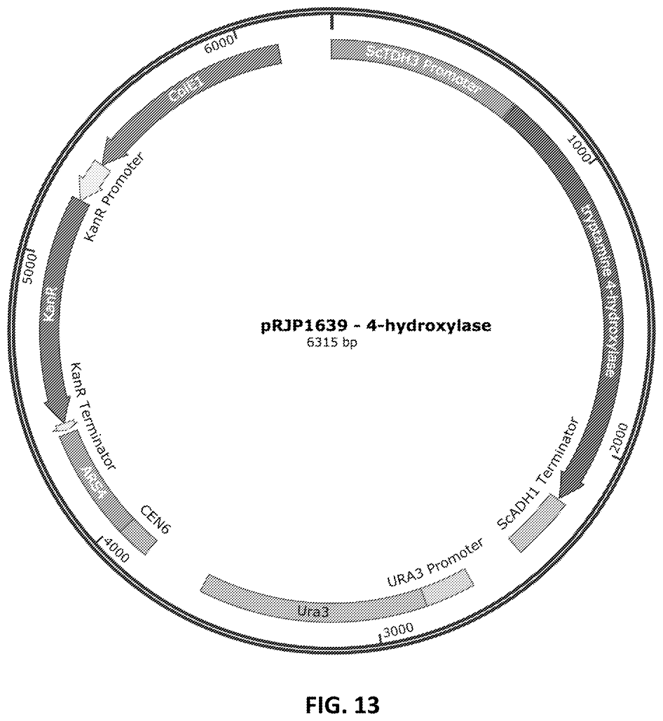

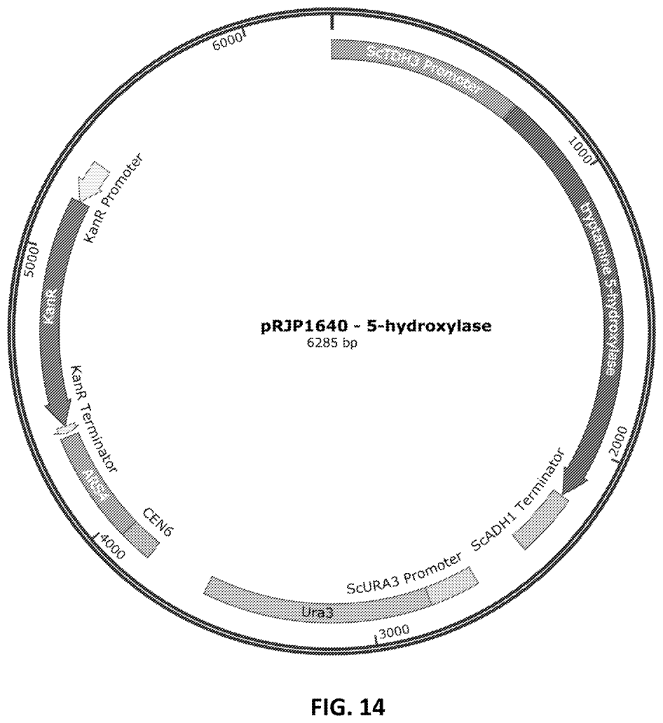

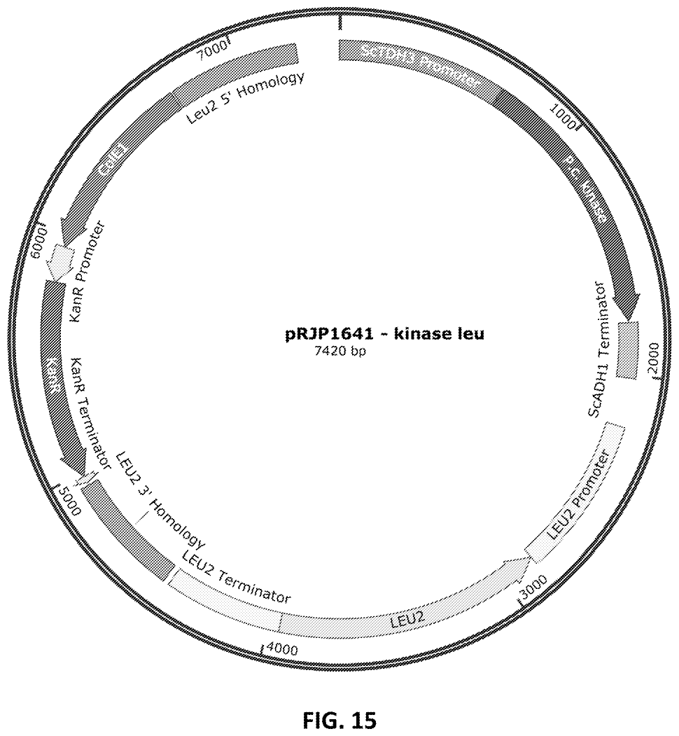

Microbes can be genetically modified to express metabolic enzymes capable of derivatizing tryptamines. Hydroxyl and phosphoryloxy substitutions to indole positions of tryptamines was investigated by expressing heterologous enzymes in yeast and feeding tryptamines with various amine substitutions. A single gene expression plasmid with CEN6/ARS4 replication sequences, URA3 expression cassette, and kanamycin resistance was cloned to contain a coding sequence for tryptamine 4-hydroxylase from P. cyanescens (SEQ ID NO: 33) and was named pRJP1639 (FIG. 13). A single gene expression plasmid with CEN6/ARS4 replication sequences, URA3 expression cassette, and kanamycin resistance was cloned to contain a coding sequence for tryptamine 5-hydroxylase from S. mansoni (SEQ ID NO: 47) and was named pRJP1640 (FIG. 14). A single gene expression plasmid with CEN6/ARS4 replication sequences, LEU2 expression cassette, and kanamycin resistance was cloned to contain a coding sequence for 4-hydroxytryptamine kinase from P. cubensis (SEQ ID NO: 41) and was named pRJP1641 (FIG. 15).

S. cerevisiae strain BY4741 (MATa his3Δ1 leu2Δ0 met15Δ0 ura3Δ0) was used for experiments in this study and was propagated at 30° C. The pRJP1639 plasmid was transformed into BY4741 by lithium acetate protocol and selected for on synthetic complete medium lacking uracil. The resulting strain, yNAB003, was isolated and genotyped. The pRJP1640 plasmid was transformed into BY4741 by lithium acetate protocol and selected for on synthetic complete medium lacking uracil. The resulting strain, yNAB004, was isolated and genotyped. The plasmid pRJP1641 was linearized by NotI digestion and transformed into strain yNAB003 by lithium acetate protocol and selected for on synthetic complete medium lacking leucine. The resulting strain, yNAB005, was isolated and genotyped.

Overnight grown cultures of yNAB003, yNAB004, and yNAB005 were used to inoculate 250 mL cultures of synthetic complete medium and were grown at 30° C. in a rotary shaker at 225 rpm. Cultures were concentrated into 25 mL of synthetic complete medium lacking uracil and transferred to 5 mL culture tubes. 5 mM of various tryptamines (including 5-methoxy-N,N-dimethyltryptamine, N,N-diisopropyl-tryptamine, N-methyl-N-isopropyltryptamine, N,N-dimethyltryptamine, N,N-tetramethylenetryptamine, and N,N-dipropyltryptamine) were added to separate tubes (FIG. 16). After 3 days of incubation at 30° C., media from cultures were sampled by centrifuging 1 mL of culture at 18000 rpm and transferring clarified media to sample vials. Analysis was performed by chromatography/mass spectrometry (LCMS) with a 1260 Infinity LC System connected to a 6120 Quadrupole Mass Spectrometer (Agilent Technologies). Zorbax Eclipse Plus C18 guard column (4.6 cm×12.5 cm, 5 μm packing, Agilent Technologies) was connected to a Zorbax Eclipse Plus C18 column (4.6 mm×100 mm, 3.5 μm packing, Agilent Technologies) at 20° C. using a 0.5 mL/min flow rate. Water and acetonitrile mobile phases contained 0.1% formic acid as the pH modifier. The elution gradient (water:acetonitrile volume ratio) was as follows: 98:2 (0-2 min), linear ramp from 98:2 to 5:95 (2-17 min), 5:95 (17-22 min), linear ramp from 5:95 to 98:2 (22-23 min), and 98:2 (23-28 min). Absorbance was measured using a diode array detector for UV—Vis analysis. MS was conducted in atmospheric pressure ionization-positive electrospray (API-ES positive) mode at 100-V fragmentor voltage with ion detection set to both full scanning mode (50-1200 m/z). Detection of tryptamines was conducted by extraction of ion masses of corresponding tryptamine not found in the unfed control sample or the original tryptamine chemical stock. 4-hydroxy derivatives of fed tryptamines were identified for the yNAB003 culture. 5-hydroxy derivatives of fed tryptamines were identified for the yNAB004 culture. Ion masses for 4-hydroxy-N,N-dipropyltryptamine and 4-phosphoryloxy-N,N-dipropyltryptamine were identified in the yNABOO5 culture media fed with N,N-dipropyltryptamine (see FIG. 16).

### Example 6. Colorimetric Screening for High Hydroxylation Activity

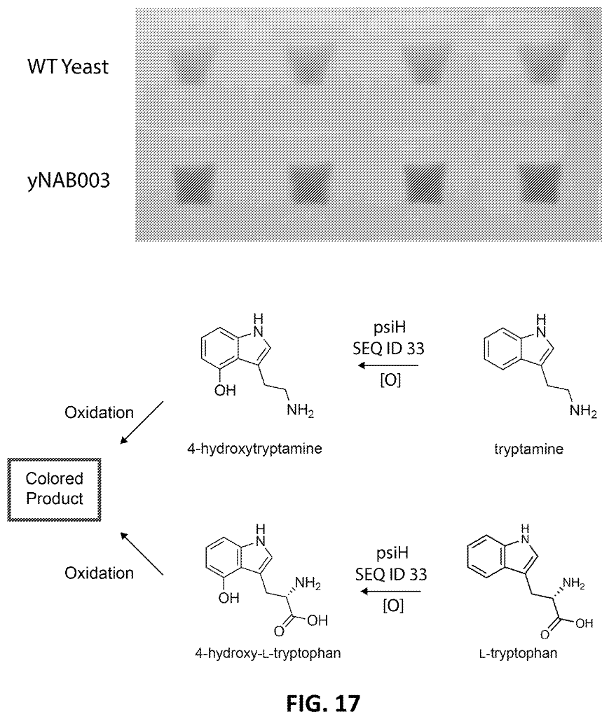

Without addition of a protecting group at the hydroxyl position of 4-hydroxy-tryptamine and 4-hydroxytryptophan, the compounds will oxidize to a colored compound. Accordingly, subsequent hydroxylase and oxidation activity on tryptamine and tryptophan can be used as an indicator of hydroxylase activity. To demonstrate this activity, color formation of the strain yNAB003 with high tryptamine 4-hydroxylase from P. cyanescens (SEQ ID NO: 33) was compared to activity of WT BY4741. Four separate 3 mL cultures were started for WT BY4741 and yNAB003 for 3 days at 30° C. in synthetic complete media with 4 mM added tryptamine at 750 rpm of high frequency shaking. After culturing, these cultures were centrifuged to pellet the cells for observation of pigment formation. The formation of blue product was observed in the yNAB003 cultures as an indication of tryptamine 4-hydroxylase activity and was not observed in the WT BY4741 cultures (FIG. 17).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-93. (canceled)

94. A method of synthesizing a tryptamine derivative, the method comprising cultivating an engineered microbial cell comprising one or more nucleic acid sequences encoding one or more of:
(i) a tryptamine 4-hydroxylase comprising an amino acid sequence having at least 80% sequence identity to any one of SEQ ID NOS: 32-35;
(ii) a tryptamine 5-hydroxylase comprising an amino acid sequence having at least 80% sequence identity to SEQ ID NO: 47;
(iii) a kinase comprising an amino acid sequence having at least 80% sequence identity to any one of SEQ ID NOS: 41-44;
(iv) a P450 reductase comprising an amino acid sequence having at least 80% sequence identity to any one of SEQ ID NOS: 36-40; and
(v) a transferase comprising an amino acid sequence having at least 80% sequence identity to any one of SEQ ID NOS: 21-31 or 46,
in the presence of a substituted tryptamine to produce the tryptamine derivative.

95. The method of claim 94, wherein the engineered microbial cell is a eukaryotic cell.

96. The method of claim 95, wherein the eukaryotic cell is a yeast cell.

97. The method of claim 96, wherein the yeast cell is of the species Saccharomyces cerevisiae.

98. The method of claim 94, wherein the engineered microbial cell is a prokaryotic cell.

99. The method of claim 98, wherein the prokaryotic cell is a bacterial cell.

100. The method of claim 99, wherein the bacterial cell is of the species Escherichia coli or Corynebacterium glutamicum.

101. The method of claim 94, wherein the substituted tryptamine is produced biosynthetically by the engineered microbial cell.

102. The method of claim 94, wherein the engineered microbial cell secretes the tryptamine derivative into a culture broth.

103. The method of claim 94, wherein the engineered microbial cell is a lysate of the engineered microbial cell.

104. The method of claim 94, wherein the tryptamine derivative is: 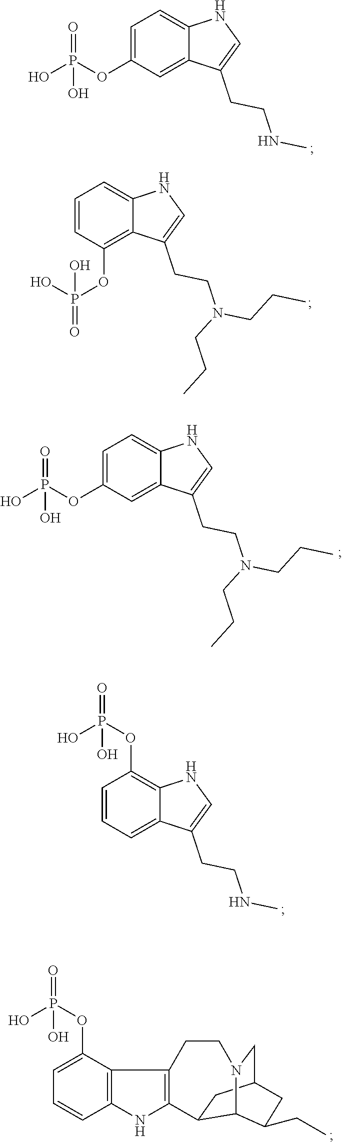 [SMILES: CNCCc1c[nH]c2ccc(OP(=O)(O)O)cc12 ; CCCN(CCC)CCc1c[nH]c2cccc(OP(=O)(O)O)c12 ; CCCN(CCC)CCc1c[nH]c2ccc(OP(=O)(O)O)cc12 ; CNCCc1c[nH]c2c(OP(=O)(O)O)cccc12 ; CCC1CC2CC3c4[nH]c5cccc(OP(=O)(O)O)c5c4CCN(C2)C13] [chemical structure]


SEQUENCE LISTING (49 sequences)

SEQ ID NO: 1 - DNA, 4969 bp, Escherichia coli
  source 1..4969: mol_type = genomic DNA
atggctgacattctgctgctcgataatatcgactcttttacgtacaacctggcagatcagttgcgcagcaatgggcataacgtggtgatttaccgcaaccatattccggcgcaaaccttaattgaacgcctggcgaccatgagcaatccggtgctgatgctttctcctggccccggtgtgccgagcgaagccggttgtatgccggaactcctcacccgcttgcgtggcaagctgcccattattggcatttgcctcggacatcaggcgattgtcgaagcttacgggggctatgtcggtcaggcgggcgaaattctccacggtaaagcctccagcattgaacatgacggtcaggcgatgtttgccggattaacaaacccgctgccggtggcgcgttatcactcgctggttggcagtaacattccggccggtttaaccatcaacgcccattttaatggcatggtgatggcagtacgtcacgatgcggatcgcgtttgtggattccagttccatccggaatccattctcaccacccagggcgctcgcctgctggaacaaacgctggcctgggcgcagcagaaactagagccagccaacacgctgcaaccgattctggaaaaactgtatcaggcgcagacgcttagccaacaagaaagccaccagctgttttcagcggtggtgcgtggcgagctgaagccggaacaactggcggcggcgctggtgagcatgaaaattcgcggtgagcacccgaacgagatcgccggggcagcaaccgcgctactggaaaacgcagcgccgttcccgcgcccggattatctgtttgctgatatcgtcggtactggcggtgacggcagcaacagtatcaatatttctaccgccagtgcgtttgtcgccgcggcctgtgggctgaaagtggcgaaacacggcaaccgtagcgtctccagtaaatctggttcgtccgatctgctggcggcgttcggtattaatcttgatatgaacgccgataaatcgcgccaggcgctggatgagttaggtgtatgtttcctctttgcgccgaagtatcacaccggattccgccacgcgatgccggttcgccagcaactgaaaacccgcaccctgttcaatgtgctggggccattgattaacccggcgcatccgccgctggcgttaattggtgtttatagtccggaactggtgctgccgattgccgaaaccttgcgcgtgctggggtatcaacgcgcggcggtggtgcacagcggcgggatggatgaagtttcattacacgcgccgacaatcgttgccgaactgcatgacggcgaaattaaaagctatcagctcaccgcagaagactttggcctgacaccctaccaccaggagcaactggcaggcggaacaccggaagaaaaccgtgacattttaacacgtttgttacaaggtaaaggcgacgccgcccatgaagcagccgtcgctgcgaacgtcgccatgttaatgcgcctgcatggccatgaagatctgcaagccaatgcgcaaaccgttcttgaggtactgcgcagtggttccgcttacgacagagtcaccgcactggcggcacgagggtaaatgatgcaaaccgttttagcgaaaatcgtcgcagacaaggcgatttgggtagaagcccgcaaacagcagcaaccgctggccagttttcagaatgaggttcagccgagcacgcgacatttttatgatgcgctacagggtgcgcgcacggcgtttattctggagtgcaagaaagcgtcgccgtcaaaaggcgtgatccgtgatgatttcgatccagcacgcattgccgccatttataaacattacgcttcggcaatttcggtgctgactgatgagaaatattttcaggggagctttaatttcctccccatcgtcagccaaatcgccccgcagccgattttatgtaaagacttcattatcgacccttaccagatctatctggcgcgctattaccaggccgatgcctgcttattaatgctttcagtactggatgacgaccaatatcgccagcttgccgccgtcgctcacagtctggagatgggggtgctgaccgaagtcagtaatgaagaggaacaggagcgcgccattgcattgggagcaaaggtcgttggcatcaacaaccgcgatctgcgtgatttgtcgattgatctcaaccgtacccgcgagcttgcgccgaaactggggcacaacgtgacggtaatcagcgaatccggcatcaatacttacgctcaggtgcgcgagttaagccacttcgctaacggttttctgattggttcggcgttgatggcccatgacgatttgcacgccgccgtgcgccgggtgttgctgggtgagaataaagtatgtggcctgacgcgtgggcaagatgctaaagcagcttatgacgcgggcgcgatttacggtgggttgatttttgttgcgacatcaccgcgttgcgtcaacgttgaacaggcgcaggaagtgatggctgcggcaccgttgcagtatgttggcgtgttccgcaatcacgatattgccgatgtggtggacaaagctaaggtgttatcgctggcggcagtgcaactgcatggtaatgaagaacagctgtatatcgatacgctgcgtgaagctctgccagcacatgttgccatctggaaagcattaagcgtcggtgaaaccctgcccgcccgcgagtttcagcacgttgataaatatgttttagacaacggccagggtggaagcgggcaacgttttgactggtcactattaaatggtcaatcgcttggcaacgttctgctggcggggggcttaggcgcagataactgcgtggaagcggcacaaaccggctgcgccggacttgattttaattctgctgtagagtcgcaaccgggcatcaaagacgcacgtcttttggcctcggttttccagacgctgcgcgcatattaaggaaaggaacaatgacaacattacttaacccctattttggtgagtttggcggcatgtacgtgccacaaatcctgatgcctgctctgcgccagctggaagaagcttttgtcagtgcgcaaaaagatcctgaatttcaggctcagttcaacgacctgctgaaaaactatgccgggcgtccaaccgcgctgaccaaatgccagaacattacagccgggacgaacaccacgctgtatctcaagcgtgaagatttgctgcacggcggcgcgcataaaactaaccaggtgctggggcaggcgttgctggcgaagcggatgggtaaaaccgaaatcatcgccgaaaccggtgccggtcagcatggcgtggcgtcggcccttgccagcgccctgctcggcctgaaatgccgtatttatatgggtgccaaagacgttgaacgccagtcgcctaacgtttttcgtatgcgcttaatgggtgcggaagtgatcccggtgcatagcggttccgcgacgctgaaagatgcctgtaacgaggcgctgcgcgactggtccggtagttacgaaaccgcgcactatatgctgggcaccgcagctggcccgcatccttatccgaccattgtgcgtgagtttcagcggatgattggcgaagaaaccaaagcgcagattctggaaagagaaggtcgcctgccggatgccgttatcgcctgtgttggcggcggttcgaatgccatcggcatgtttgctgatttcatcaatgaaaccaacgtcggcctgattggtgtggagccaggtggtcacggtatcgaaactggcgagcacggcgcaccgctaaaacatggtcgcgtgggtatctatttcggtatgaaagcgccgatgatgcaaaccgaagacgggcagattgaagaatcttactccatctccgccggactggatttcccgtctgtcggcccacaacacgcgtatcttaacagcactggacgcgctgattacgtgtctattaccgatgatgaagcccttgaagccttcaaaacgctgtgcctgcacgaagggatcatcccggcgctggaatcctcccacgccctggcccatgcgttgaaaatgatgcgcgaaaacccggataaagagcagctactggtggttaacctttccggtcgcggcgataaagacatcttcaccgttcacgatattttgaaagcacgaggggaaatctgatggaacgctacgaatctctgtttgcccagttgaaggagcgcaaagaaggcgcattcgttcctttcgtcacgctcggtgatccgggcattgagcagtcattgaaaattatcgatacgctaattgaagccggtgctgacgcgctggagttaggtatccccttctccgacccactggcggatggcccgacgattcaaaacgccactctgcgcgcctttgcggcaggtgtgactccggcacaatgttttgaaatgctggcactgattcgccagaaacacccgaccattcccattggcctgttgatgtatgccaatctggtgtttaacaaaggcattgatgagttttatgcccagtgcgaaaaagtcggcgtcgattcggtgctggttgccgatgtgccagttgaagagtccgcgcccttccgccaggccgcgttgcgtcataatgtcgcacctatcttcatctgcccgccaaatgccgatgacgacctgctgcgccagatagcctcttacggtcgtggttacacctatttgctgtcacgagcaggcgtgaccggcgcagaaaaccgcgccgcgttacccctcaatcatctggttgcgaagctgaaagagtacaacgctgcacctccattgcagggatttggtatttccgccccggatcaggtaaaagcagcgattgatgcaggagctgcgggcgcgatttctggttcggccattgttaaaatcatcgagcaacatattaatgagccagagaaaatgctggcggcactgaaagtttttgtacaaccgatgaaagcggcgacgcgcagttaa

SEQ ID NO: 2 - DNA, 4390 bp, Bacillus subtilis
  source 1..4390: mol_type = genomic DNA
atgaacagatttctacaattgtgcgttgacggaaaaacccttactgccggtgaggctgaaacgctgatgaatatgatgatggcagcggaaatgactccttctgaaatgggggggatattgtcaattcttgctcatcggggggagacgccagaagagcttgcgggttttgtgaaggcaatgcgggcacacgctcttacagtcgatggacttcctgatattgttgatacatgcggaacagggggagacggtatttccacttttaatatctcaacggcctcggcaattgttgcctcggcagctggtgcgaaaatcgctaagcatggcaatcgctctgtctcttctaaaagcggaagcgctgatgttttagaggagctagaggtttctattcaaaccactcccgaaaaggtcaaaagcagcattgaaacaaacaacatgggatttctttttgcgccgctttaccattcgtctatgaaacatgtagcaggtactagaaaagagctaggtttcagaacggtatttaatctgcttgggccgctcagcaatcctttacaggcgaagcgtcaggtgattggggtctattctgttgaaaaagctggactgatggcaagcgcactggagacgtttcagccgaagcacgttatgtttgtatcaagccgtgacggtttagatgagctttcaattacagcaccgaccgacgtgattgaattaaaggacggagagcgccgggagtataccgtttcacccgaagatttcggtttcacaaatggcagacttgaagatttacaggtgcagtctccgaaagagagcgcttatctcattcagaatatttttgaaaataaaagcagcagttccgctttatctattacggcttttaatgcgggtgctgcgatttacacggcgggaattaccgcctcactgaaggaaggaacggagctggcgttagagacgattacaagcggaggcgctgccgcgcagcttgaacgactaaagcagaaagaggaagagatctatgcttgaaaaaatcatcaaacaaaagaaagaagaagtgaaaacactggttctgccggtagagcagcctttcgagaaacgttcatttaaggaggcgctggcaagcccgaatcggtttatcgggttgattgccgaagtgaagaaagcatcgccgtcaaaagggcttattaaagaggattttgtacctgtgcagattgcaaaagactatgaggctgcgaaggcagatgcgatttccgttttaacagacaccccgttttttcaaggggaaaacagctatttatcagacgtaaagcgtgctgtttcgattcctgtacttagaaaagattttattgattctcttcaagtagaggaatcaagaagaatcggagcggatgccatattgttaatcggcgaggtgcttgatcccttacaccttcatgaattatatcttgaagcaggtgaaaaggggatggacgtgttagtggaggttcatgatgcatcaacgctagaacaaatattgaaagtgttcacacccgacattctcggcgtaaataatcgaaacctaaaaacgtttgaaacatctgtaaagcagacagaacaaatcgcatctctcgttccgaaagaatccttgcttgtcagcgaaagcggaatcggttctttagaacatttaacatttgtcaatgaacatggggcgcgagctgtacttatcggtgaatcattgatgagacaaacttctcagcgtaaagcaatccatgctttgtttagggagtgaggttgtgaagaaaccggcattaaaatattgcggtattcggtcactaaaggatttgcagcttgcggcggaatcacaggctgattacctaggatttatttttgctgaaagcaaacgaaaagtatctccggaagatgtgaaaaaatggctgaaccaagttcgtgtcgaaaaacaggttgcaggtgtttttgttaatgaatcaatagagacgatgtcacgtattgccaagagcttgaagctcgacgtcattcagcttcacggtgatgaaaaaccggcggatgctgctgctcttcgcaagctgacaggctgtgaaatatggaaggcgcttcaccatcaagataacacaactcaagaaatagcccgctttaaagataatgttgacggctttgtgattgattcatctgtaaaagggtctagaggcggaactggtgttgcattttcttgggaatgtgtgccggaatatcagcaggcggctattggtaaacgctgctttatcgctggcggcgtgaatccggatagcatcacacgcctattgaaatggcagccagaaggaattgaccttgccagcggaattgaaaaaaacggacaaaaagatcagaatctgatgaggcttttagaagaaaggatgaaccgatatgtatccatatccgaatgaaataggcagatacggtgattttggcggaaagtttgttccggaaacactcatgcagccgttagatgaaatacaaacagcatttaaacaaatcaaggatgatcccgcttttcgtgaagagtattataagctgttaaaggactattccggacgcccgactgcattaacatacgctgatcgagtcactgaatacttaggcggcgcgaaaatctatttgaaacgagaagatttaaaccatacaggttctcataaaatcaataatgcgctaggtcaagcgctgcttgctaaaaaaatgggcaaaacgaaaatcattgctgaaaccggtgccggccagcatggtgttgccgctgcaacagttgcagccaaattcggcttttcctgtactgtgtttatgggtgaagaggatgttgcccgccagtctctgaacgttttccgcatgaagcttcttggagcggaggtagtgcctgtaacaagcggaaacggaacattgaaggatgccacaaatgaggcgatccggtactgggttcagcattgtgaggatcacttttatatgattggatcagttgtcggcccgcatccttatccgcaagtggtccgtgaatttcaaaaaatgatcggagaggaagcgaaggatcagttgaaacgtattgaaggcactatgcctgataaagtagtggcatgtgtaggcggaggaagcaatgcgatgggtatgtttcaggcatttttaaatgaagatgttgaactgatcggcgctgaagcagcaggaaaaggaattgatacacctcttcatgccgccactatttcgaaaggaaccgtaggggttattcacggttcattgacttatctcattcaggatgagttcgggcaaattattgagccctactctatttcagccggtctcgactatcctggaatcggtccggagcatgcatatttgcataaaagcggccgtgtcacttatgacagtataaccgatgaagaagcggtggatgcattaaagcttttgtcagaaaaagaggggattttgccggcaatcgaatctgcccatgcgttagcgaaagcattcaaactcgccaaaggaatggatcgcggtcaactcattctcgtctgtttatcaggccggggagacaaggatgtcaacacattaatgaatgtattggaagaagaggtgaaagcccatgtttaaattggatcttcaaccatcagaaaaattgtttatcccgtttattacggcgggcgatccagttcctgaggtttcgattgaactggcgaagtcactccaaaaagcaggcgccacagcattggagcttggtgttgcatactctgacccgcttgcagacggtccggtgatccagcgggcttcaaagcgggcgcttgatcaaggaatgaatatcgtaaaggcaatcgaattaggcggagaaatgaaaaaaaacggagtgaatattccgattatcctctttacgtattataatcctgtgttacaattgaacaaagaatactttttcgctttactgcgggaaaatcatattgacggtctgcttgttccggatctgccattagaagaaagcaacagccttcaagaggaatgtaaaagccatgaggtgacgtatatttctttagttgcgccgacaagcgaaagccgtttgaaaaccattattgaacaagccgaggggttcgtctactgtgtatcttctctgggtgtgaccggtgtccgcaatgagttcaattcatccgtgtacccgttcattcgtactgtgaagaatctcagcactgttccggttgctgtagggttcggtatatcaaaccgtgaacaggtcataaagatgaatgaaattagtgacggtgtcgtagtgggaagtgcgctcgtcagaaaaatagaagaattaaaggaccggctcatcagcgctgaaacgagaaatcaggcgctgcaggagtttgaggattatgcaatggcgtttagcggcttgtacagtttaaaatga

SEQ ID NO: 3 - DNA, 5467 bp, Lactococcus lactis
  source 1..5467: mol_type = genomic DNA
atgaaaacaaggagtcatcaaatgaaaaatgaacttgaaaaagtgatgtcaggtcgtgacatgaccgaaaatgaaatgaatatgcttgctaattcaattatccaaggtgaattaagcgaggtccaaattgccagctttttagtagcattaaaaatgaaaggtgaagcagcaagcgaattgactggtttggctcgagctttacaaaaagcagcgattcccattccaacaaatttgacaaatgcgatggacaattgtggaacaggaggcgaccgctcattcagttttaatatttcaaccacagccgcttttgttttagcagctggtggagtcaatatggcaaaacacggaaatcgctccattaccagtaaatctggctcggcagacgttcttgaggccttaggaatcaatctttatttaccagcagaaaagttagctcaagtttttgacaaagttggtttagttttcctttttgctcaaaatctccacccagcgatgaaatacttcacgccagtccgcagacaactcgaaattccaacaattatgaacttgactgggccactaatcaatccaattccacttgatacgcaacttcttggtacctcacgtccagatttacttgaattaacagcaaatgttttgaaaggcttgggccgtaagcgagcattagtcatcacaggtgaaggcggaatggacgaagcaactccctttggacttaatcattacgcacttttagaaaatgacaaagtgactttgcatgaatttagagcctcagaagttggtatttcagaagttcaactcaatgatattcgtggaggtgaagccccagaaaatgctgaaattttaaaaaatgtccttgaaaatcaaccgtcagcctttttagaaacgaccgttttaaatgccggacttggattttatgccaatggaaaagttgattccatcaaatccggagttgaccttgcaagagaagtaattagtacaggagcagctcttaccaagttgcatgaattacaagcagaacaaattggttaaaaatcttgatggcaaattttgaaatagcagaaaatgagagaaaaatatgaacataaaaaaaggaaaatttctagaaacaatcctagcagaaaaacgacttgaaattgctaaaatgccagaagaacaagtaggaaaagttcgtcaaacatacaatttttatgattacttaaaagaacattccgaccagcttcaagtgattgccgaagtcaaaaaagcttcgcccagtctaggtgatattaacttagaagtggatatcgttgaccaagccaaaaattacgaacaagccggtgccgctatgatttccgtcttaactgaccctgtattttttaaaggaaatattgaatatctctgtgaaatttcagaaaatgtccaaatccccaccttgaacaaggattttatcatcgataaaaaacaaatcaatcgggcagttaatgcgggagcaacagttattttactcattgtcgcagtttttgaaaatcaataccccaaactccaaaacctctacaactacgcactttcactaggacttgaagttcttgttgaaacacataataaagcagaacttgagattgctcatcagcttggagctaaaattattggagttaataatcgtaatttaaaaacctttgaagtgatcttacaaaattcagtagatttgacaccctactttaaagaagacagtatctacatttccgaatcaggcatttttagcgcaaacgaagcccaaaaagtttccgatactttcaatggaatattggttggaacagcactcatgcaatcagaaaatctagaaaaatctttgaaagatttaaaagtcaagaggaaaacgaatgaaaattaaaatctgtggcttatctacaaaagaagctgttgatacagctgtagaatctggtgtcacacatctcggttttattcttagtccctcaaaacgccaagttgcaccagaaaaaattcttcaaatcacaaacgatgtcccaaaaacagtcaaaaaagtaggagtttttgttgatgaacctattgattttgtaaaaaaagccattcaagttgctcaactcgatctggttcagcttcacggaaatgaagatatgaattacattaatcaactagatatttcggttattaaagcaataagaccagaccaagaatttaaagaatacgaagatgtaattttattatttgatagtccacaagctggaagtggtcaagcatttgattgggactctttggtgaccagcggtctgaaaaataaatttttcatcgctggtggacttaatccagaaaatgtagcagctgctattcaacattttccaaatgcctacggtgtggatgtttcttctggagtagaaactgacggaattaaaaaccttacaaaaataaaaaactttgttcaaaatgcaagccttgcctcatcaaagcaattatttatagaatttttaagaatcacaaaaaagctaaatgaaaataagattatcccttatttaatgggaagtttagcagttgagcaaataatcaattttccaacaaatcctgatgacattgatattcaactcaaaacgtctgattttgaaaattttgagcaattaacaagtttaatggaaaaattaggttatcagcttattgacttacatgagcataaatttgaaaaagctagtattcatgttggctttgcaagtgtggagacccttaaaaactatgccggggttgactatttgaccattcaacaagaaagaatggaaaatggcgaaaaatatcatcttccaaatgttgaacaatcccttaaaatctatgaggcagcaaaacgagatgagtggcgaggagggaagcaaaaagattcctttattttcgatgagttaataaaggaacagaagaggaatgacaatgaatgataatcttattgaagagggtgtagagattcgaaatggtctcattattaagtcaattcaaaaagaagatatattagagctttggcaaattagttatggacctaaatctgatttacattggatgtctttcaacgctccctattttgaggagccaatcctgagttgggaagaattttcaagaaaaatatctcttaaaataaattaaccaaatgttgcacttattatctttcaaaatcgaatcattggaatgctgtcagcttattgggaagacggtaaattacaaaaatggcttgagtttggtatagtgatttatgatagtaaattgtggggacgtggaattggacaggatgccttatctttttggttgaagcacctttttgaaacttatccgaagattcagcacataggatttacaacttggtcaggaaatcaaggaatgatgagactaggagaaaaaagtggtctaaaacttgaagggcaaatcagaaaagttagatattggcaagaaacttggtatgattcaataaaatatggaattttaagagaagaactaaaaaaataaataaaaaaaatcaaaggagcaacaacatgacctacaaccaacctaacaacaaaggattttacggccaattcgggggccaattcgtacctgagacactaatgacagcagtaaaacaattagaagaagcctacgtagatagtaaaaaagaccctctctttcaagcagaacttaaagaattacttaaagactatgttggacgagaaaacccactctattatgcaaaacgcttaacagaatatgcgggcggagcaaaaatttatcttaaaagagaagacctaaaccatacaggagcacacaaaattaacaatgccctcggacaagtcctccttgccaaaaaaatgggaaaaaataaagtcattgctgaaacaggtgcaggccaacacggtgtcgcaagcgcaaccgcggctgccctctttggcatggaatgtacgatttatatgggtgaagaagacgttaaaagacaatctctcaatgtctttcgcatggaattactcggggcaaaagttcattcagtaactgatggttcacgcgtacttaaagatgcggttaatgcagcacttagagcatgggttgctcaagttgaagatacgcattatgtaatgggctcagttcttggaccacatccatttccacaaattgtgcgtgattatcaagctgttattggacaggaagcgcgtgcccaatttttagaaaaagaaaataaacttccagatgctttagtagcttgtgtcggtggaggttcaaattctatgggacttttttatcccttcgttaatgatgaatcagttgccatgtatggtgttgaagccgctggccttgggattgatacaccacatcatgcggcaacaattactaaaggccgccccggtgttcttcacggaacactcatggatgtccttcaagatgaaaatggtcaaatgttagaagcctttagtatttcagccggtttagactatccaggaatcggaccagaacactcttatttcaatgctgttggacgagcaaaatatgttgatattacagatgaagaagcacttgaaggttttaaaatcttatctagaactgaaggaattatcccagcactagaaagttctcatgctatcgcctatgcagtcaaattagcaaaagaattaggagcagataaatcaatgattgtttgtctttcaggacgtggagataaggatgtggttcaagttaaagaacgacttgaagcagaaaaagaggtgaaaaaatgaaaactttacaaatgactttaagcaataaaaaaaataattttattccttatatcatggctggcgaccatgaaaaaggcttagaaggtcttaaagaaaccattcaactgcttgagcaagctgggagttccgctattgaaattggcgttccattttcagatccggttgctgatggtccagtcatcgaacaagcaggtttgcgtgcgttagcaagaaatgtatcactttcaagtattcttgaaaccttaaaaacaattgatacaaaagttcctctagtaattatgacctatttcaatcccgtttatcagtttggaattgaaaagtttgttgcagctcttgaaaaaacaccagttaaaggccttatcattcctgatttgcctaaagaacatgaggactatatcaaaccatttatcaatgataaagatatctgtttagttcctctggtctcattaaccacgccactttctcggcaaaaagaacttgtagccgatgctgaaggatttatctatgccgttgcaataaatggagtaactgggaaagaaaatgcttatagtaaccagcttgaccaacatttaaaagcgttatcttcattaacggatgttcctgttttgacaggatttggaatttctacattatctgatgtggaccgttttaataaagtgtcctcaggagttattgttggttcaaaaattgttcgtgatttacatgaaggtaaagaaaacgaagttattaaatttattgaaaacgcaatcaatttttaa

SEQ ID NO: 4 - DNA, 4573 bp, Corynebacterium glutamicum
  source 1..4573: mol_type = genomic DNA
atgacttctccagcaacactgaaagttctcaacgcctacttggataaccccactccaaccctggaggaggcaattgaggtgttcaccccgctgaccgtgggtgaatacgatgacgtgcacatcgcagcgctgcttgccaccatccgtactcgcggtgagcagttcgctgatattgccggcgctgccaaggcgttcctcgcggcggctcgtccgttcccgattactggcgcaggtttgctagattccgctggtactggtggcgacggtgccaacaccatcaacatcaccaccggcgcatccctgatcgcagcatccggtggagtgaagctggttaagcacggcaaccgttcggtgagctccaagtccggctccgccgatgtgctggaagcgctgaatattcctttgggccttgatgtggatcgtgctgtgaagtggttcgaagcgtccaacttcaccttcctgttcgcacctgcgtacaaccctgcgattgcgcatgtgcagccggttcgccaggcgctgaaattccccaccatcttcaacacgcttggaccattgctgtccccggcgcgcccggagcgtcagatcatgggcgtggccaatgccaatcatggacagctcatcgccgaggtcttccgcgagttgggccgtacacgcgcgcttgttgtgcatggcgcaggcaccgatgagatcgcagtccacggcaccaccttggtgtgggagcttaaagaagacggcaccatcgagcattacaccatcgagcctgaggaccttggccttggccgctacacccttgaggatctcgtaggtggcctcggcactgagaacgccgaagctatgcgcgctactttcgcgggcaccggccctgatgcacaccgtgatgcgttggctgcgtccgcaggtgcgatgttctacctcaacggcgatgtcgactccttgaaagatggtgcacaaaaggcgctttccttgcttgccgacggcaccacccaggcatggttggccaagcacgaagagatcgattactcagaaaaggagtcttccaatgactagtaataatctgcccacagtgttggaaagcatcgtcgagggtcgtcgcggacacctggaggaaattcgcgctcgcatcgctcacgtggatgtggatgcgcttccaaaatccacccgttctctgtttgattccctcaaccagggtaggggaggggcgcgtttcatcatggagtgcaagtccgcatcgccttctttgggaatgattcgtgagcactaccagccgggtgaaatcgctcgcgtgtactctcgctacgccagcggcatttccgtgctgtgcgagccggatcgttttggtggcgattacgatcacctcgctaccgttgccgctacctctcatcttccggtgctgtgcaaagacttcatcattgatcctgtccaggtacacgcggcgcgttactttggtgctgatgccatcctgctcatgctctctgtgcttgatgatgaagagtacgcagcactcgctgccgaggctgcgcgttttgatctggatatcctcaccgaggttattgatgaggaggaagtcgcccgcgccatcaagctgggtgcgaagatctttggcgtcaaccaccgcaacctgcatgatctgtccattgatttggatcgttcacgtcgcctgtccaagctcattccagcagatgccgtgctcgtgtctgagtctggcgtgcgcgataccgaaaccgtccgccagctaggtgggcactccaatgcattcctcgttggctcccagctgaccagccaggaaaacgtcgatctggcagcccgcgaattagtctacggccccaacaaagtctgcggactcacctcaccaagtgcagcacaaaccgctcgcgcagcgggtgcggtctacggcgggctcatcttcgaagaggcatcgccacgcaatgtttcacgtgaaacattgcaaaaaatcatcgccgcagagcccaacctgcgctacgtcgcggtcagccgtcgcacctccgggtacaaggatttgcttgtcgacggcatcttcgccgtacaaatccacgccccactgcaggacagcgtcgaagcagaaaaggcattgatcgccgccgttcgtgaagaggttggaccgcaggtccaggtctggcgcgcgatctcgatgtccagccccttgggggctgaagtggcagctgcggtggagggtgacgtcgataagctaattcttgatgcccatgaaggtggcagcggggaagtattcgactgggctacggtgccggccgctgtgaaggcaaagtctttgctcgcgggaggcatctctccggacaacgctgcgcaggcactcgctgtgggctgcgcaggtttggacatcaactctggcgtggaataccccgccggtgcaggcacgtgggctggggcgaaagacgccggcgcgctgctgaaaattttcgcgaccatctccacattccattactaaaggtttaaataggatcatgactgaaaaagaaaacttgggcggctccacgctgctgcctgcatacttcggtgaattcggcggccagttcgtcgcggaatccctcctgcctgctctcgaccagctggagaaggccttcgttgacgcgaccaacagcccagagttccgcgaagaactcggcggctacctccgcgattacctcggccgcccaaccccgctgaccgaatgctccaacctgccactcgcaggcgaaggcaaaggctttgcgcggatcttcctcaagcgcgaagacctcgtccacggcggtgcacacaaaactaaccaggtgatcggccaggtgctgcttgccaagcgcatgggcaaaacccgcatcatcgcagagaccggcgcaggccagcacggcaccgccaccgctctcgcatgtgcgctcatgggcctcgagtgcgttgtctacatgggcgccaaggacgttgcccgccagcagcccaacgtctaccgcatgcagctgcacggcgcgaaggtcatccccgtggaatctggttccggcaccctgaaggacgccgtgaatgaagcgctgcgcgattggaccgcaaccttccacgagtcccactaccttctcggcaccgccgccggcccgcacccattcccaaccatcgtgcgtgaattccacaaggtgatctctgaggaagccaaggcacagatgctagagcgcaccggcaagcttcccgacgttgtggtcgcctgtgtcggtggtggctccaacgccatcggcatgttcgcagacttcattgacgatgaaggtgtagagctcgtcggcgctgagccagccggtgaaggcctcgactccggcaagcacggcgcaaccatcaccaacggtcagatcggcatcctgcacggcacccgttcctacctgatgcgcaactccgacggccaagtggaagagtcctactccatctccgccggacttgattacccaggcgtcggcccacagcacgcacacctgcacgccaccggccgcgccacctacgttggtatcaccgacgccgaagccctccaagcattccagtacctcgcccgctacgaaggcatcatccccgcactggaatcctcacacgcgttcgcctacgcactcaagcgcgccaagaccgccgaagaggaaggccagaacttaaccatcctcgtctccctatccggccgtggcgacaaggacgttgaccacgtgcgccgcaccctcgaagaaaatccagaactgatcctgaaggacaaccgatgagccgttacgacgatctttttgcacgcctcgacacggcaggggagggcgcctttgttcccttcatcatgctgagcgacccttcaccagaggaggctttccagatcatctccacagcaatcgaagctggcgcagatgcactggaacttggcgtacctttctccgacccagttgccgatggccccaccgtcgcggaatcccacctccgcgcactcgacggcggcgccaccgtagacagcgcactcgagcagatcaagcgcgtgcgcgcagcctacccagaggttcccatcggaatgctcatctacggcaacgttcctttcacccgtggcttggatcgcttctaccaagagttcgctgaagctggcgcagactccatcctcctgccagacgtcccagtccgcgaaggcgcaccgttttctgcagcagctgcagcagccggaattgatcccatttacatcgctccggccaacgccagcgagaaaaccctcgagggtgtctccgccgcatcaaagggctacatctacgccatctcccgcgacggcgtcaccggcaccgaacgtgaatcatccaccgacggcctgtccgcagtggtggacaacatcaagaaatttgatggcgcacccatcctcttgggcttcggcatctcatcccctcagcacgtggcagacgcgattgcagcgggtgcttccggtgcgatcacgggttccgcgatcaccaagatcattgcttcccactgcgaaggtgagcacccgaacccgtccaccattcgagatatggacggtttgaagaaggatctcactgagttcatctctgcgatgaaggcagcgaccaagaaggtttag

SEQ ID NO: 5 - protein, 531 aa, Escherichia coli
MADILLLDNIDSFTYNLADQLRSNGHNVVIYRNHIPAQTLIERLATMSNPVLMLSPGPGVPSEAGCMPELLTRLRGKLPIIGICLGHQAIVEAYGGYVGQAGEILHGKASSIEHDGQAMFAGLTNPLPVARYHSLVGSNIPAGLTINAHFNGMVMAVRHDADRVCGFQFHPESILTTQGARLLEQTLAWAQQKLEPANTLQPILEKLYQAQTLSQQESHQLFSAVVRGELKPEQLAAALVSMKIRGEHPNEIAGAATALLENAAPFPRPDYLFADIVGTGGDGSNSINISTASAFVAAACGLKVAKHGNRSVSSKSGSSDLLAAFGINLDMNADKSRQALDELGVCFLFAPKYHTGFRHAMPVRQQLKTRTLFNVLGPLINPAHPPLALIGVYSPELVLPIAETLRVLGYQRAAVVHSGGMDEVSLHAPTIVAELHDGEIKSYQLTAEDFGLTPYHQEQLAGGTPEENRDILTRLLQGKGDAAHEAAVAANVAMLMRLHGHEDLQANAQTVLEVLRSGSAYDRVTALAARG

SEQ ID NO: 6 - protein, 348 aa, Corynebacterium glutamicum
MTSPATLKVLNAYLDNPTPTLEEAIEVFTPLTVGEYDDVHIAALLATIRTRGEQFADIAGAAKAFLAAARPFPITGAGLLDSAGTGGDGANTINITTGASLIAASGGVKLVKHGNRSVSSKSGSADVLEALNIPLGLDVDRAVKWFEASNFTFLFAPAYNPAIAHVQPVRQALKFPTIFNTLGPLLSPARPERQIMGVANANHGQLIAEVFRELGRTRALVVHGAGTDEIAVHGTTLVWELKEDGTIEHYTIEPEDLGLGRYTLEDLVGGLGTENAEAMRATFAGTGPDAHRDALAASAGAMFYLNGDVDSLKDGAQKALSLLADGTTQAWLAKHEEIDYSEKESSND

SEQ ID NO: 7 - protein, 348 aa, Corynebacterium glutamicum
MTSPATLKVLNAYLDNPTPTLEEAIEVFTPLTVGEYDDVHIAALLATIRTRGEQFADIAGAAKAFLAAARPFPITGAGLLDSAGTGGDGANTINITTGASLIAASGGVKLVKHGNRSVSSKSGSADVLEALNIPLGLDVDRAVKWFEAFNFTFLFAPAYNPEIAHVQPVRQALKFPTIFNTLGPLLSPARPERQIMGVANANHGQLIAEVFRELGRTRALVVHGAGTDEIAVHGTTLVWELKEDGTIEHYTIEPEDLGLGRYTLEDLVGGLGTENAEAMRATFAGTGPDAHRDALAASAGAMFYLNGDVDSLKDGAQKALSLLADGTTQAWLAKHEEIDYSEKESSND

SEQ ID NO: 8 - protein, 452 aa, Escherichia coli
MQTVLAKIVADKAIWVETRKEQQPLASFQNEVQPSTRHFYDALQGARTAFILECKKASPSKGVIRDDFDPARIAAIYKHYASAISVLTDEKYFQGSFDFLPIVSQIAPQPILCKDFIIDPYQIYLARYYQADACLLMLSVLDDEQYRQLAAVAHSLEMGVLTEVSNEEELERAIALGAKVVGINNRDLRDLSIDLNRTRELAPKLGHNVTVISESGINTYAQVRELSHFANGFLIGSALMAHDDLNAAVRRVLLGENKVCGLTRGQDAKAAYDAGAIYGGLIFVATSPRCVNVEQAQEVMAAAPLQYVGVFRNHDIADVADKAKVLSLAAVQLHGNEDQLYIDNLREALPAHVAIWKALSVGETLPARDFQHIDKYVFDNGQGGSGQRFDWSLLNGQTLGNVLLAGGLGADNCVEAAQTGCAGLDFNSAVESQPGIKDARLLASVFQTLRAY

SEQ ID NO: 9 - protein, 474 aa, Corynebacterium glutamicum
MTSNNLPTVLESIVEGRRGHLEEIRARIAHVDVDALPKSTRSLFDSLNQGRGGARFIMECKSASPSLGMIREHYQPGEIARVYSRYASGISVLCEPDRFGGDYDHLATVAATSHLPVLCKDFIIDPVQVHAARYFGADAILLMLSVLDDEEYAALAAEAARFDLDILTEVIDEEEVARAIKLGAKIFGVNHRNLHDLSIDLDRSRRLSKLIPADAVLVSESGVRDTETVRQLGGHSNAFLVGSQLTSQENVDLAARELVYGPNKVCGLTSPSAAQTARAAGAVYGGLIFEEASPRNVSRETLQKIIAAEPNLRYVAVSRRTSGYKDLLVDGIFAVQIHAPLQDSVEAEKALIAAVREEVGPQVQVWRAISMSSPLGAEVAAAVEGDVDKLILDAHEGGSGEVFDWATVPAAVKAKSLLAGGISPDNAAQALAVGCAGLDINSGVEYPAGAGTWAGAKDAGALLKIFATISTFHY

SEQ ID NO: 10 - protein, 397 aa, Escherichia coli [sequence omitted]

SEQ ID NO: 11 - protein, 417 aa, Corynebacterium glutamicum
MTEKENLGGSTLLPAYFGEFGGQFVAESLLPALDQLEKAFVDATNSPEFREELGGYLRDYLGRPTPLTECSNLPLAGEGKGFARIFLKREDLVHGGAHKTNQVIGQVLLAKRMGKTRIIAETGAGQHGTATALACALMGLECVVYMGAKDVARQQPNVYRMQLHGAKVIPVESGSGTLKDAVNEALRDWTATFHESHYLLGTAAGPHPFPTIVREFHKVISEEAKAQMLERTGKLPDVVVACVGGGSNAIGMFADFIDDEGVELVGAEPAGEGLDSGKHGATITNGQIGILHGTRSYLMRNSDGQVEESYSISAGLDYPGVGPQHAHLHATGRATYVGITDAEALQAFQYLARYEGIIPALESSHAFAYALKRAKTAEEEGQNLTILVSLSGRGDKDVDHVRRTLEENPELILKDNR

SEQ ID NO: 12 - protein, 520 aa, Escherichia coli
MQTQKPTLELLTCEGAYRDNPTALFHQLCGDRPATLLLESADIDSKDDLKSLLLVDSALRITALGDTVTIQALSGNGEALLALLDNALPAGVESEQSPNCRVLRFPPVSPLLDEDARLCSLSVFDAFRLLQNLLNVPKEEREAMFFGGLFSYDLVAGFEDLPQLSAENNCPDFCFYLAETLMVIDHQKKSTRIQASLFAPNEEEKQRLTARLNELRQQLTEAAPPLPVVSVPHMRCECNQSDEEFGGVVRLLQKAIRAGEIFQVVPSRRFSLPCPSPLAAYYVLKKSNPSPYMFFMQDNDFTLFGASPESSLKYDATSRQIEIYPIAGTRPRGRRADGSLDRDLDSRIELEMRTDHKELSEHLMLVDLARNDLARICTPGSRYVADLTKVDRYSYVMHLVSRVVGELRHDLDALHAYRACMNMGTLSGAPKVRAMQLIAEAEGRRRGSYGGAVGYFTAHGDLDTCIVIRSALVENGIATVQAGAGVVLDSVPQSEADETRNKARAVLRAIATAHHAQETF

SEQ ID NO: 13 - protein, 280 aa, Corynebacterium glutamicum
MSRYDDLFARLDTAGEGAFVPFIMLSDPSPEEAFQIISTAIEAGADALELGVPFSDPVADGPTVAESHLRALDGGATVDSALEQIKRVRAAYPEVPIGMLIYGNVPFTRGLDRFYQEFAEAGADSILLPDVPVREGAPFSAAAAAAGIDPIYIAPANASEKTLEGVSAASKGYIYAISRDGVTGTERESSTDGLSAVVDNIKKFDGAPILLGFGISSPQHVADAIAAGASGAITGSAITKIIASHCEGEHPNPSTIRDMDGLKKDLTEFISAMKAATKKV

SEQ ID NO: 14 - protein, 439 aa, Psilocybe cubensis
MQVIPACNSAAIRSLCPTPESFRNMGWLSVSDAVYSEFIGELATRASNRNYSNEFGLMQPIQEFKAFIESDPVVHQEFIDMFEGIQDSPRNYQELCNMFNDIFRKAPVYGDLGPPVYMIMAKLMNTRAGFSAFTRQRLNLHFKKLFDTWGLFLSSKDSRNVLVADQFDDRHCGWLNERALSAMVKHYNGRAFDEVFLCDKNAPYYGFNSYDDFFNRRFRNRDIDRPVVGGVNNTTLISAACESLSYNVSYDVQSLDTLVFKGETYSLKHLLNNDPFTPQFEHGSILQGFLNVTAYHRWHAPVNGTIVKIINVPGTYFAQAPSTIGDPIPDNDYDPPPYLKSLVYFSNIAARQIMFIEADNKEIGLIFLVFIGMTEISTCEATVSEGQHVNRGDDLGMFHFGGSSFALGLRKDCRAEIVEKFTEPGTVIRINEVVAALKA

SEQ ID NO: 15 - protein, 439 aa, Psilocybe cyanescens
MQVLPACQSSALKTLCPSPEAFRKLGWLPTSDEVYNEFIDDLTGRTCNEKYSSQVTLLKPIQDFKTFIENDPIVYQEFISMFEGIEQSPTNYHELCNMFNDIFRKAPLYGDLGPPVYMIMARIMNTQAGFSAFTKESLNFHFKKLFDTWGLFLSSKNSRNVLVADQFDDKHYGWFSERAKTAMMINYPGRTFEKVFICDEHVPYHGFTSYDDFFNRRFRDKDTDRPVVGGVTDTTLIGAACESLSYNVSHNVQSLDTLVIKGEAYSLKHLLHNDPFTPQFEHGSIIQGFLNVTAYHRWHSPVNGTIVKIVNVPGTYFAQAPYTIGSPIPDNDRDPPPYLKSLVYFSNIAARQIMFIEADNKDIGLIFLVFIGMTEISTCEATVCEGQHVNRGDDLGMFHFGGSSFALGLRKDSKAKILEKFAKPGTVIRINELVASVRK

SEQ ID NO: 16 - protein, 436 aa, Psilocybe cyanescens
MQVLTACYTSTLKSLLPSFDAFRSMGWLPVSDKTYNEWIGDLRSRASDKNYTSQVGLIQPIKDFKAFIESDPVVHQEFITMFEGIEESPRNYEELCHMFNDIFRKAPVYGDLGPPVYMVMARIMNTQAGFSAFTKQSLNSHFKRLFDTWGVFLSSKESRYVLVTDQFDDNHYGWLSDRAKSAMVKHYYGRTFEQVFICDEHAPYHGFQSYDDFFNRRFRDRDIDRPVVGGIENTTLISAACESLSYNVCHDLQSLDTLFVKGESYSLKHLLNDDPFARQFEHGSILQGFLNVTAYHRWHAPVNGTILKIINVPGTYFAQAPHTIGDSLDSDHPPYLKSLAYFSNIAARQIMFIEADNKDIGLIFLVFIGMTEISTCEATVSEGQHVNRGDDLGMFHFGGSSFALGLRKDCKAEIFERFAEQGTVIKINEVVAAVKD

SEQ ID NO: 17 - protein, 430 aa, Gymnopilus dilepis
MAKTLRPTAQAFRELGWLPASDGVYNKFMKDLTNRASNENHLCHVALLQPIQDFKTFIENDPVVYQEFVCMFEGIEESPRNYHELCNMFNEIFRRAPYYGDLGPPVYMAMAKIMNTRAGFSAFTRESLNFHFKRLFDTWGLFLSSPASRDVLVADKFDSKHYGWFSEPAKAAMMAQYDGRTFEQVFICDETAPYHGFKSYDDFFNRKFRAMDIDRPVVGGIANTTLIGSPCEALSYNVSDDVHSLETLYFKGEGYSLRHLLHDDPSTEQFEHGSIIQGFLNITGYHRWHAPVSGTIMKIVDVPGTYFAQAPSTIGDPFPVNDYDPQAPYLRSLAYFSNIAARQIIFIQADNEDIGLIYLILIGMTEVSTCEALVCPGQHVERGDDLGMFHFGGSSFALGLRKNSKAAILEELKTQGTVIKVNDVIAAVQA

SEQ ID NO: 18 - protein, 480 aa, Homo sapiens
MNASEFRRRGKEMVDYMANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 19 - protein, 480 aa, Bacillus atrophaeus
MSENLQLSAEEMRQLGYQAVDLIIDHMNHLKSKPVSETIDSDILRNKLTESIPENGSDPKELLHFLNRNVFNQITHVDHPHFLAFVPGPNNYVGVVADFLASGFNVFPTAWIAGAGAEQIELTTINWLKSMLGFPDSAEGLFVSGGSMANLTALTVARQAKLNNDIENAVVYFSDQTHFSVDRALKVLGFKHHQICRIETDEHLRISVSALKKQIKEDRTKGKKPFCVIANAGTTNCGAVDSLNELADLCNDEDVWLHADGSYGAPAILSEKGSAMLQGIHRADSLTLDPHKWLFQPYDVGCVLIRNSQYLSKTFRMMPEYIKDSETNVEGEINFGECGIELSRRFRALKVWLSFKVFGVAAFRQAIDHGIMLAEQVEAFLGKAKDWEVVTPAQLGIVTFRYIPSELASTDTINEINKKLVKEITHRGFAMLSTTELKEKVVIRLCSINPRTTTEEMLQIMMKIKALAEEVSISYPCVAE

SEQ ID NO: 20 - protein, 500 aa, Catharanthus roseus
MGSIDSTNVAMSNSPVGEFKPLEAEEFRKQAHRMVDFIADYYKNVETYPVLSEVEPGYLRKRIPETAPYLPEPLDDIMKDIQKDIIPGMTNWMSPNFYAFFPATVSSAAFLGEMLSTALNSVGFTWVSSPAATELEMIVMDWLAQILKLPKSFMFSGTGGGVIQNTTSESILCTIIAARERALEKLGPDSIGKLVCYGSDQTHTMFPKTCKLAGIYPNNIRLIPTTVETDFGISPQVLRKMVEDDVAAGYVPLFLCATLGTTSTTATDPVDSLSEIANEFGIWIHVDAAYAGSACICPEFRHYLDGIERVDSLSLSPHKWLLAYLDCTCLWVKQPHLLLRALTTNPEYLKNKQSDLDKVVDFKNWQIATGRKFRSLKLWLILRSYGVVNLQSHIRSDVAMGKMFEEWVRSDSRFEIVVPRNFSLVCFRLKPDVSSLHVEEVNKKLLDMLNSTGRVYMTHTIVGGIYMLRLAVGSSLTEEHHVRRVWDLIQKLTDDLLKEA

SEQ ID NO: 21 - protein, 309 aa, Psilocybe cubensis
MHIRNPYRTPIDYQALSEAFPPLKPFVSVNADGTSSVDLTIPEAQRAFTAALLHRDFGLTMTIPEDRLCPTVPNRLNYVLWIEDIFNYTNKTLGLSDDRPIKGVDIGTGASAIYPMLACARFKAWSMVGTEVERKCIDTARLNVVANNLQDRLSILETSIDGPILVPIFEATEEYEYEFTMCNPPFYDGAADMQTSDAAKGFGFGVGAPHSGTVIEMSTEGGESAFVAQMVRESLKLRTRCRWYTSNLGKLKSLKEIVGLLKELEISNYAINEYVQGSTRRYAVAWSFTDIQLPEELSRPSNPELSSLF

SEQ ID NO: 22 - protein, 312 aa, Psilocybe cyanescens
MHIRNPYRSPIDYQALVEAFPPLRPYVTVNQDNTTSIDLTVPEVQRLYTAALLHRDFGLVIDLPEDRLCPTLLTRTPRLNYVLWVEDILKVTNTALGLSEDRPVKGIDIGTGAAAIYPMLACARFKTWSMIGTEIDRKCIDTARVNVLTNNLQDRLSIIETSIDGPILVPIFEATTDYEYDFTMCNPPFYDGAADMQTSDAAKGFGFGVNAPHSGTVIEMSTEGGESAFVAQMVRESLDHRTRCRWFTSNLGKLKSLHEIVGLLREHQISNYAINEYVQGTTRRYAIAWSFTNIRLPEDLTRPSNPELSSLF

SEQ ID NO: 23 - protein, 317 aa, Psilocybe cyanescens
MHNRNPYRDVIDYQALAEAYPPLKPHVTVNADNTASIDLTIPEVQRQYTAALLHRDFGLTITLPEDRLCPTVPNRLNYVLWIEDIFQCTNKALGLSDDRPVKGVDIGTGASAIYPMLACARFKQWSMIATEVERKCIDTARLNVLANNLQDRLSILEVSVDGPILVPIFDTFERATSDYEFEFTMCNPPFYDGAADMQTSDAAKGFGFGVNAPHSGTVIEMATEGGEAAFVAQMVRESMKLQTRCRWFTSNLGKLKSLHEIVALLRESQITNYAINEYVQGTTRRYALAWSFTDIKLTEELYRPSNPELGPLCSTFV

SEQ ID NO: 24 - protein, 309 aa, Gymnopilus dilepis
MHIRNPYLTPPDYEALAEAFPALKPYVTVNPDKTTTIDFAIPEAQRLYTAALLYRDFGLTITLPPDRLCPTVPNRLNYVLWIQDILQITSAALGLPEARQVKGVDIGTGAAAIYPILGCSLAKNWSMVGTEVEQKCIDIARQNVISNGLQDRITITANTIDAPILLPLFEGDSNFEWEFTMCNPPFYDGAADMETSQDAKGFGFGVNAPHTGTVVEMATDGGEAAFVSQMVRESLHLKTRCRWFTSNLGKLKSLHEIVGLLREHQITNYAINEYVQGTTRRYAIAWSFTDLRLSDHLPRPPNPDLSALF

SEQ ID NO: 25 - protein, 263 aa, Homo sapiens
MKGGFTGGDEYQKHFLPRDYLATYYSFDGSPSPEAEMLKFNLECLHKTFGPGGLQGDTLIDIGSGPTIYQVLAACDSFQDITLSDFTDRNREELEKWLKKEPGAYDWTPAVKFACELEGNSGRWEEKEEKLRAAVKRVLKCDVHLGNPLAPAVLPLADCVLTLLAMECACCSLDAYRAALCNLASLLKPGGHLVTTVTLRLPSYMVGKREFSCVALEKEEVEQAVLDAGFDIEQLLHSPQSYSVTNAANNGVCFIVARKKPGP

SEQ ID NO: 26 - protein, 209 aa, Acinetobacter sp.
MIYKFYQQHIFPHLLNQVMQTPSLMDQRRQLLLPIAGDVLEIGFGTGVNLPFYQNVETLYALEPNADLYQLAAKRIHESTIHVQHIQAYAEKLPFADASLDHIVSTWTLCSIENLAQALIEMYRVLKPNGTLHLVEHVQYQDNAKLQHLQNLLTPIQKRLADGCHLNRNIEQALRDAHFDFTEQHYFAAQGIPKLAQRMFFARAQKQPE

SEQ ID NO: 27 - protein, 445 aa, Oryza sativa
MEITSSAMLKTTTTPPHPLAGEKVPLSAFDRAAFDVFVPLVFAYRAPAPSSEAVKEGLRVAVAAYPLVSGRIAVDGQGRRRRRRVLHVNNEGVLVLDATVEVDLDAVLAANVATDLYPALPEHSFGAALLQVQLTRFGCGGLVVGLIGHHHVFDGHSMSTFCATWARAVRDSEAFIVPSPSLDRAITGVPRSPPAPVFDHRSIEFKVGNKSSDSSGAAAAAAVEKIANIGVRFTAKFVAELKARVGGRCSTFECVLAHAWKKITAARGLKPEEFTRVRVAVNCRRRANPPAPADLFGNMVLWAFPRLQVRRLLSSSYRDVVGAIRAAVARVDAEYIQSFVDYVEVADARGEELAATAAEPGETLCPDLEVDSWLGFRFHEMDLGTGPPAAVLSPDLPIEGLMILVPVGGDGGGVDLFVALADDHAQAFEQICYSLEEHAMIHSHL

SEQ ID NO: 28 - protein, 207 aa, Homo sapiens
MSTQSTHPLKPEAPRLPPGIPESPSCQRRHTLPASEFRCLTPEDAVSAFEIEREAFISVLGVCPLYLDEIRHFLTLCPELSLGWFEEGCLVAFIIGSLWDKERLMQESLTLHRSGGHIAHLHVLAVHRAFRQQGRGPILLWRYLHHLGSQPAVRRAALMCEDALVPFYERFSFHAVGPCAITVGSLTFMELHCSLRGHPFLRRNSGC

SEQ ID NO: 29 - protein, 275 aa, Drosophila melanogaster
MEVQKLPDQSLISSMMLDSRCGLNDLYPIARLTQKMEDALTVSGKPAACPVDQDCPYTIELIQPEDGEAVIAMLKTFFFKDEPLNTFLDLGECKELEKYSLKPLPDNCSYKAVNKKGEIIGVFLNGLMRRPSPDDVPEKAADSCEHPKFKKILSLMDHVEEQFNIFDVYPDEELILDGKILSVDTNYRGLGIAGRLTERAYEYMRENGINVYHVLCSSHYSARVMEKLGFHEVFRMQFADYKPQGEVVFKPAAPHVGIQVMAKEVGPAKAAQTKL

SEQ ID NO: 30 - protein, 209 aa, Danio rerio
MMAPQVVSSPFLKPFFLKTPISVSSPRRQRRHTLPASEFRNLTPQDAISVFEIEREAFISVSGECPLTLDEVLVFLGQCPELSMGWFEEGQLVAFIIGSGWDKEKLEQEAMSTHVPDSPTVHIHVLSVHRHCRQQGKGSILLWRYLQYLRCLPGLRRALLVCEEFLVPFYQKAGFKEKGPSAISVAALTFTEMEYQLGGLAYARRNSGC

SEQ ID NO: 31 - protein, 436 aa, Oryza sativa
MAAVTVEITRSEVLRPSPASAGGGEMVPLTVFDRAATDGYIPTMFAWDAAAAAALSNDAIKDGLAAVLSRFPHLAGRFAVDERGRKCFRLNNAGARVLEASAAGDLADALAHDVAAHVNQLYPQADKDRVDEPLLQVQLTRYTCGGLVIGAVSHHQVADGQSMSVFFTEWAAAVRTAGAALPTPFLDRSAVAAPRIPPAPAFDHRNVEFRGEGSRSHSYGALPLERMRNLAVHFPPEFVAGLKARVGGARCSTFQCLLAHAWKKITAARDLSPKEYTQVRVAVNCRGRAGPAVPTDYFGNMVLWAFPRMQVRDLLSASYAAVVGVIRDAVARVDERYIQSFVDFGEVAAGDELAPTAAEPGTAFCPDLEVDSWIGFRFHDLDFGGGPPCAFLPPDVPIDGLLIFVPSCAAKGGVEMFMALDDQHVEALRQICYSMD

SEQ ID NO: 32 - protein, 508 aa, Psilocybe cubensis
MIAVLFSFVIAGCIYYIVSRRVRRSRLPPGPPGIPIPFIGNMFDMPEESPWLTFLQWGRDYNTDILYVDAGGTEMVILNTLETITDLLEKRGSIYSGRLESTMVNELMGWEFDLGFITYGDRWREERRMFAKEFSEKGIKQFRHAQVKAAHQLVQQLTKTPDRWAQHIRHQIAAMSLDIGYGIDLAEDDPWLEATHLANEGLAIASVPGKFWVDSFPSLKYLPAWFPGAVFKRKAKVWREAADHMVDMPYETMRKLAPQGLTRPSYASARLQAMDLNGDLEHQEHVIKNTAAEVNVGGGDTTVSAMSAFILAMVKYPEVQRKVQAELDALTNNGQIPDYDEEDDSLPYLTACIKELFRWNQIAPLAIPHKLMKDDVYRGYLIPKNTLVFANTWAVLNDPEVYPDPSVFRPERYLGPDGKPDNTVRDPRKAAFGYGRRNCPGIHLAQSTVWIAGATLLSAFNIERPVDQNGKPIDIPADFTTGFFRHPVPFQCRFVPRTEQVSQSVSGP

SEQ ID NO: 33 - protein, 507 aa, Psilocybe cyanescens
MIVLLVSLVLAGCIYYANARRVRRSRLPPGPPGIPLPFIGNMFDMPSESPWLRFLQWGRDYHTDILYLNAGGTEIIILNTLDAITDLLEKRGSMYSGRLESTMVNELMGWEFDLGFITYGERWREERRMFAKEFSEKNIRQFRHAQIKAANQLVRQLIKTPDRWSQHIRHQIAAMSLDIGYGIDLAEDDPWIAATQLANEGLAEASVPGSFWVDSFPALKYLPSWLPGAGFKRKAKVWKEGADHMVNMPYETMKKLTVQGLARPSYASARLQAMDPDGDLEHQEHVIRNTATEVNVGGGDTTVSAVSAFILAMVKYPEVQRQVQAELDALTSKGVVPNYDEEDDSLPYLTACVKEIFRWNQIAPLAIPHRLIKDDVYRGYLIPKNALVYANSWAVLNDPEEYPNPSEFRPERYLSSDGKPDPTVRDPRKAAFGYGRRNCPGIHLAQSTVWIAGATLLSVFNIERPVDGNGKPIDIPATFTTGFFRHPEPFQCRFVPRTQEILKSVSG

SEQ ID NO: 34 - protein, 483 aa, Psilocybe cyanescens
MINLPLSLVLVGCVYYIVSRRIRRSRLPPGPPGIPIPFVGNMYDMPSESPWLTFLQWGREYNDRGLTTIFRVESTMVNKLMGWEFDLGFITYGDRWREERRMFSKEFSEKAIKQFRHSQVKAAHRFVQQLAANGEPSRLPHYIRHQIAAMSLDIGYGVDLAQDDPWLEAAHLANEGLATASVPGTFWIDSFPALKYLPSWFPGAGFKRQAKIWKEAADHMVNMPYERMKKLAPQGLARPSYASARLQAMDPNGDLEYQEQVIKNTASQVNVGGGDTTVSAVSAFILAMVIYPEVQRKVQAELDAVLSNGRIPDYDEENDSMPYLTACVKELFRWNQIAPLAIPHKLVKDDIYRGYLIPKNTLVFANSWAVLNDPEVYPDPSVFRPERYLGPDGKPNDTVRDPRKAAFGYGRRNCPGIHLALSTVWITAATLLSVFDIERPVDHKGNPIDIPAAFTKGFFRHPEPFQCRFVPRNEDSLKSLSGL

SEQ ID NO: 35 - protein, 563 aa, Gymnopilus dilepis
MQGNPAVLLLLLTLTLCVYYAHSRRARRARLPPGPPGIPLPFVGNLFDMPSNSPWLTYLQWGETYQTDIIYLNAGGTEMVILNTLEAITDLLEKRGSIYSGRFESTMVNELMGWDFDLGFITYGERWREERRMFSKEFNEKNIKQFRHAQIRAANLLVGQLTKTPERWHQLIRHQIAAMSLDIGYGIDLLEGDPWLEATQLANEGLAIASVPGSFWVDSLPILKYMPSWFPGAEFKRKAKVWRESTDHMINMPYEKMKKLMVQDLVRPSYASARLQEMDPNGDLQHQEHVIRNTAMEVNVGGADTTVSAVAAFILAMVKYPDVQRKVQAELDAVGCRDELPEFDEDNDALPYLTACVKEIFRWNQVAPLAIPHRLDKDDHYRGYIIPKNALVFANTWAVLNDPSVYPDPSEFRPERYLGPDGKPDPRIRDPRKAAFGYGRRACPGIHLAQSTVWIVGATLLSVFDIERPMDANGKPIDIPAAFTTGFFRYSIHDCLVVETMHPANTVCVDIPNPSDADSFLVPKRLSNPHPIIDLPSRNPACQEDGVVALSNAWRSTLPVQDV

SEQ ID NO: 36 - protein, 691 aa, Saccharomyces cerevisiae
MPFGIDNTDFTVLAGLVLAVLLYVKRNSIKELLMSDDGDITAVSSGNRDIAQVVTENNKNYLVLYASQTGTAEDYAKKFSKELVAKFNLNVMCADVENYDFESLNDVPVIVSIFISTYGEGDFPDGAVNFEDFICNAEAGALSNLRYNMFGLGNSTYEFFNGAAKKAEKHLSAAGAIRLGKLGEADDGAGTTDEDYMAWKDSILEVLKDELHLDEQEAKFTSQFQYTVLNEITDSMSLGEPSAHYLPSHQLNRNADGIQLGPFDLSQPYIAPIVKSRELFSSNDRNCIHSEFDLSGSNIKYSTGDHLAVWPSNPLEKVEQFLSIFNLDPETIFDLKPLDPTVKVPFPTPTTIGAAIKHYLEITGPVSRQLFSSLIQFAPNADVKEKLTLLSKDKDQFAVEITSKYFNIADALKYLSDGAKWDTVPMQFLVESVPQMTPRYYSISSSSLSEKQTVHVTSIVENFPNPELPDAPPVVGVTTNLLRNIQLAQNNVNIAETNLPVHYDLNGPRKLFANYKLPVHVRRSNFRLPSNPSTPVIMIGPGTGVAPFRGFIRERVAFLESQKKGGNNVSLGKHILFYGSRNTDDFLYQDEWPEYAKKLDGSFEMVVAHSRLPNTKKVYVQDKLKDYEDQVFEMINNGAFIYVCGDAKGMAKGVSTALVGILSRGKSITTDEATELIKMLKTSGRYQEDVW

SEQ ID NO: 37 - protein, 695 aa, Aspergillus niger
MAQLDTLDLVVLAVLLVGSVAYFTKGTYWAVAKDPYASTGPAMNGAAKAGKTRNIIEKMEETGKNCVIFYGSQTGTAEDYASRLAKEGSQRFGLKTMVADLEEYDYENLDQFPEDKVAVFVLATYGEGEPTDNAVEFYQFFTGDDVAFESGASADEKPLSKLKYVAFGLGNNTYEHYNAMVRQVDAAFQKLGAQRIGSAGEGDDGAGTMEEDFLAWKEPMWAALSESMDLQEREAVYEPVFCVTENESLSPEDESVYLGEPTQSHLQGTPKGPYSAHNPFIAPIAESRELFTVKDRNCLHMEISIAGSNLSYQTGDHIAVWPTNAGAEVDRFLQVFGLEGKRDSVINIKGIDVTAKVPIPTPTTYDAAVRYYMEVCAPVSRQFVATLAAFAPDEESKAEIVRLGSDKDYFHEKVTNQCFNIAQALQSITSKPFSAVPFSLLIEGITKLQPRYYSISSSSLVQKDKISITAVVESVRLPGASHMVKGVTTNYLLALKQKQNGDPSPDPHGLTYSITGPRNKYDGIHVPVHVRHSNFKLPSDPSRPIIMVGPGTGVAPFRGFIQERAALAAKGEKVGPTVLFFGCRKSDEDFLYKDEWKTYQDQLGDNLKIITAFSREGPQKVYVQHRLREHSELVSDLLKQKATFYVCGDAANMAREVNLVLGQIIAAQRGLPAEKGEEMVKHMRSSGSYQEDVWS

SEQ ID NO: 38 - protein, 1351 aa, Psilocybe cyanescens
MTDPNRTTFSSALHPLAVVSMASSSSDVFVLGLGVVLAALYIFRDQLFAASKPKVAPVSTTKPANGSANPRDFIAKMKQGKKRIVIFYGSQTGTAEEYAIRLAKEAKQKFGLASLVCDPEEYDFEKLDQLPEDSIAFFVVATYGEGEPTDNAVQLLQNLQDDSFEFSNGERKLSGLKYVVFGLGNKTYEHYNLIGRTVDAQLAKMGAVRVGERGEGDDDKSMEEDYLEWKDGMWDAFAAAMGVEEGQGGDSADFVVSELESHPPEKVYLGEYSARALTKTKGIHDAKNPLAAPITVARELFQSVVDRNCVHVEFNIEGSGITYQHGDHVGLWPLNPDVEVERLLCVLGLTEKRDAVISIESLDPALAKVPFPVPTTYAAVLRHYIDVSAVAGRQILGTLSKFAPTPEAEAFLKNLNTNKEEYHNVVANGCLKLGEILQVATGNDITVAPTPGNTTKWPIPFDIIVSAIPRLQPRYYSISSSPKVHPNTIHATVVVLKYENVPTDPIPRKWVYGVGSNFLLNLKHAINKEPVPFITQNGEQRVGVPEYLIAGPRGSYKTESHFKAPIHVRRSTFRLPTNPKSPVIMIGPGTGVAPFRGFVQERVALARRSVEKNGPESLNDWGRISLFYGCRRSDEDFLYKDEWPQYQEELKGKFKLHCAFSRENYKPDGSKIYVQDLIWEDREHIADAILNGKGYVYICGEAKSMSKQVEEVLARILGEAKGGSGAVEGVAEIKLLKERSRLMLDYELAFRKFSQLQFARVATFAMLRSSFSLQRLFSTSSALRNVQRPIRDHLQKQDAPWEPRVAESAQSVSEEILKAQTPLQVPTNAKATTSDSRTDSREPLTAYDLQLVKKRVREWSEQAMIALRNRADDFTAHTKTTFSQLGLQLNRVTGYEEIEALKRGVVEQEERINVARQAARKAKVAYEEAVVQRSNSQREVNDLLQRKSSWMDSDVGRFTTLVRQDHLYEQEEARAKAAVEETEEAVDREFSKLLRTILARYHEEQVWSDKIRSASTYGSLAALGLNMLVFIMAIVVVEPWKRRRLAQTFERKIEELSEENGIKLDATMLSIAQQIEQQVNLIGSLKDDISRNAPVIPEPAQEVRAETEIEEETSPFVSLEFLPLSRRQLEVAAVGAGAFASNLWFGFGDDALELLMLSTRANKVPPRNLSRIHMTSFIIKAHEDRPTNSTWKQDLECAFCRIIRGELPASKVYENDKVIAILDIMPLRKGHTLVIPKAHISRLSELPSELASSVGEAVCKVAHALTQALDNTGLNVVCNQEYAQAVPHVHYHVIPAPKFGYPGHGVESTNGVVGGKAPLTHREMHQKEFEAREELDDDDAKVLLKSIRARL

SEQ ID NO: 39 - protein, 592 aa, Psilocybe cyanescens
MSVSEDDHRGLLIVYATETGNAQDAADYIARQCRRIAFQCRVVNIDSFLLPDLLSETIVIFVVSTTGSGVEPRSMTPLWTSLLRGDLPTDTFEDLYFSVFGLGDTAYEKFCWAAKKLSRRLESIGGIEFYMRGEGDEQHPLGIDGALQPWTDGLINKLLEVAPLPPGEEIKPINDVPLPRVLLKDTSKTALNHSADPLKSDLQYHKAIVKKNDRITAADWYQDVRHLVFDFQDNIQYSPGDVAVIHPVALEHDVDAFLVTMSWQNIADEPFEIEQAMYDQSLPDHLPPITTLRTLFTRFLDFNAVPRRSFFQYLRYFTSDEREQEKLDEFLSAAGADELYEYCYRVRRTIHEVLSEFRHVKIPKGYIFDVFPPLRPREFSIASSIKTHLHQIHLCVAIVKYRTKLKIPRKGVCTYYLSILKPGDTLLVGIRRGLLRLPGKNDTPVIFIGPGTGIAPMRSAIEQRIANGCHENTLYFGCRSASKDQHYGSEWQAYAANQELKYRSAFSRDGVEGEARVYVQDLIRQDSERIWDLVGHHKAWVLVSGSSNKMPAAVKDAVAYAVEKYGGLSAEEAKEYVHLMVKEGRLIEECWS

SEQ ID NO: 40 - protein, 1063 aa, Psilocybe cyanescens
MSLNGSGLLTPSSEVTLSSPSTPVLIYTFPQSNGTRPKSPVYIHIDDPGVQVSTLVEYISSQPENSSSVYIYDVAEQVGFGTSTKQWAKQGLDISPVVDLQTRAGAGLSLVGRLSQGTSIDAVKGTVLTAYTTPSGLALMAPSFAYLPVPSSTTRLIIQVPTVTPVGETLTLSPTLSPLASVWSILPENVAVLLSSSPQQTVDFATLAYKVIDSHIVHLFDHHSSAREIGRTFTPLTTIGKSGLTLQEAVKQAGYEPLEYHGDPEAKTIVVLLNSSLALSLKAAVSVGTSGLGVVVVNVLRPWDEAAIQTIIPSSATIVHVLDDVPNAVTQGSLYVDVFSALWSTTPKRSVHSHRITPSQTQKFIAAGGEFLRFVEEVTHIAVSEPSVASIKKTLFFSVPDSPLALLSRFVQELFLTKRTISSRHLTDYDVYSKPGGISAQRLLISRDKSTDNVPVQAILPLDPNSVGHSDFLGVLDHNLLKTHSLLKHAKKGSIVVVASPWTPDEFSANITYEVAEVITSRQLSVYTIDVKSIANDLELFIQEQKIEKGEAQVLLFEFVFLRFYLGAAATEQAIIQLMSVLFDDIDLTKFSAAAWLGLKPVVVALPEVTPSDSPTLKEFEANAIAVETSEGQTVVNGARLSTWHDAAKHLLFPSAFSPPTDPDSLSNPALRPEVPDTTFLVTCTVNKRLTPLEYDRNVFHLEFDTSGTGLKYAIGEALGVHGWNDEQEVLDFCEWYGVDPDRLITIPVIGSDDGKMHTRTVLQALQQQIDLFGRPPKSFYTDLAEYATVDVDRYALRFIGSPEGVSTFKKMSEKDTVSFGDVLKKYKSARPGIERLCELIGDIKPRHYSIASAQSVVGDRVDLLVVTVDWLTPEGSPRYGQCTRYLAGLKIGQKVTVSIKPSVMKLPPNLKQPLIMAGLGTGAAPFRAFLQHLAWLASKGEEIGPVFYYFGSRYQAAEYLYGEEIEAFILGGVITRAGLAFSRDGPKKVYIQHKMLEDSETLAKMLHDDDGVFYLCGPTWPVPDVYEALVNALVKYKGSDPVKAGEYLESLKEEERYVLEVY

SEQ ID NO: 41 - protein, 362 aa, Psilocybe cubensis
MAFDLKTEDGLITYLTKHLSLDVDTSGVKRLSGGFVNVTWRIKLNAPYQGHTSIILKHAQPHMSTDEDFKIGVERSVYEYQAIKLMMANREVLGGVDGIVSVPEGLNYDLENNALIMQDVGKMKTLLDYVTAKPPLATDIARLVGTEIGGFVARLHNIGRERRDDPEFKFFSGNIVGRTTSDQLYQTIIPNAAKYGVDDPLLPTVVKDLVDDVMHSEETLVMADLWSGNILLQLEEGNPSKLQKIYILDWELCKYGPASLDLGYFLGDCYLISRFQDEQVGTTMRQAYLQSYARTSKHSINYAKVTAGIAAHIVMWTDFMQWGSEEERINFVKKGVAAFHDARGNNDNGEITSTLLKESSTA

SEQ ID NO: 42 - protein, 362 aa, Psilocybe cyanescens
MAFDLKTPEGLLLYLTRHLSLDVDPSGVKRLSGGFVNVTWRIRLNAPYQGHTSIILKHAQPHLSSDEDFKIGVERSAYEYQALKVMSANQEVLGGDDSRVSVPEGLHYDVENNALIMQDVGTMKTLLDYATAKPPLSTEIASLVGTEIGAFIARLHNLGRKRRDQPAFKFFSGNIVGRTTADQLYQTIIPNAAKYGINDPLLPTVVKDLVGEVMNSEETLIMADLWSGNILLEFVEGNPSELKKIWLVDWELCKYGPASLDMGYFLGDCYLIARFQDELVGTTMRKAYLKGYARTAKGTINYSKVTASIGAHLVMWTDFMKWGNYEEREEFVKKGVEALHDAWEDNNDGEITSVLVNEASST

SEQ ID NO: 43 - protein, 362 aa, Psilocybe cyanescens
MAFDLKTVEGLIVYLTKCLSLEVDSSGVKRLSGGFVNVTWRIRLNAPYQGHTSIILKHAQPHMSTDKDFKIGVERSVYEYQALKVISANREALGGIDSRVSAPEGLHYDVENNALIMQDVGTLKTLMDYVIEKPAISTEMARLIGTEIGDFVARLHSIGRQKRDQPDFKFFSGNIVGRTTADQLYQTILPNTAKYGIDDPLLPTVVKDLVDEAMQSEETLIMADLWTGNILVEFEEGNLSVLKKIWLVDWELCKYGPVRLDMGYFLGDCFLISRFKNEQVAKAMRQAFLQRYNRVSDTPINYSVATTGIAAHIVMWTDFMNWGTEEERKEYVKKGVAGIHDGRNHNVDGEITSILMQEASTA

SEQ ID NO: 44 - protein, 360 aa, Gymnopilus dilepis
MTFDLKTEEGLLVYLTQHLSLDVDLDGLKRLSGGFVNITWRIRLNAPFKGYTNIILKHAQPHLSSDENFKIGVERSAYEYRALKIVSESPILSGDDNLVFVPQSLHYDVVHNALIVQDVGSLKTLMDYVTARPSLSSEMAKLVGGQIGAFIARLHNIGRENKDHPEFNFFSGNIVGRTTAVQLYETIVPNATKYDIDDPIIPVVVQELIEEVKGSDETLIMADLWGGNILLEFGKDSSDLGKIWVVDWELCKYGPPSLDMGYFLGDCFLLAQFQDEKVATAMRRAYLENYAKIAKVPMDYDRSTTGIGAHLVMWTDFMNWGSDEERKTSVEKGVRAFHDAKRDNKEGEIPSILLRESSRT

SEQ ID NO: 45 - protein, 622 aa, Saccharomyces cerevisiae
MLFYSFVWSVLAASVALAKTHKLNYTASWVTANPDGLHEKRMIGFNGEWPLPDIHVEKGDRVELYLTNGFQDNTATSLHFHGLFQNTSLGNQLQMDGPSMVTQCPIVPGQTYLYNFTVPEQVGTFWYHAHMGAQYGDGMRGAFIIHDPEEPFEYDHERVITLSDHYHENYKTVTKEFLSRYNPTGAEPIPQNILFNNTMNVTLDFTPGETYLFRFLNVGLFVSQYIILEDHEMSIVEVDGVYVKPNFTDSIYLSAGQRMSVLIKAKDKMPTRNYAMMQIMDETMLDVVPPELQLNQTIQMRYGHSLPEARALNIEDCDLDRATNDFYLEPLIERDLLAHYDHQIVMDVRMVNLGDGVKYAFFNNITYVTPKVPTLTTLLTSGKLASDPRIYGDNINAQLLKHNDIIEVVLNNYDSGRHPFHLHGHNFQIVQKSPGFHVDEAYDESEQDEMTVPYNESAPLQPFPERPMVRDTVVLEPSGHVVLRFRADNPGVWYFHCHVDWHLQQGLASVFIEAPVLLQEREKLNENYLDICKAADIPVVGNAAGHSNDWFDLKGLPRQPEPLPKGFTTEGYLALIISTIIGVWGLYSIAQYGIGEVIPNDEKVYHTLREILAENEIEVSRG

SEQ ID NO: 46 - protein, 207 aa, Bos taurus
MSTPSIHCLKPSPLHLPSGIPGSPGRQRRHTLPANEFRCLTPKDAAGVFEIEREAFISVSGNCPLNLDEVRHFLTLCPELSLGWFVEGRLVAFIIGSLWDEERLTQESLTLHRPGGRTAHLHALAVHHSFRQQGKGSVLLWRYLQHAGGQPAVRRAVLMCEDALVPFYQRFGFHPAGPCAVVVGSLTFTEMHCSLRGHAALRRNSDR

SEQ ID NO: 47 - protein, 499 aa, Schistosoma mansoni
MISTESDLRRQLDENVRSEADESTKEECPYINAVQSHHQNVQEMSIIISLVKNMNDMKSIISIFTDRNINILHIESRLGRLNMKKHTEKSEFEPLELLVHVEVPCIEVERLLEELKSFSSYRIVQNPLMNLPEAKNPTLDDKVPWFPRHISDLDKVSNSVLMYGKELDADHPGFKDKEYRKRRMMFADIALNYKWGQQIPIVEYTEIEKTTWGRIYRELTRLYKTSACHEFQKNLGLLQDKAGYNEFDLPQLQVVSDFLKARTGFCLRPVAGYLSARDFLSGLAFRVFYCTQYIRHQADPFYTPEPDCCHELLGHVPMLADPKFARFSQEIGLASLGTSDEEIKKLATCYFFTIEFGLCRQDNQLKAYGAGLLSSVAELQHALSDKAVIKPFIPMKVINEECLVTTFQNGYFETSSFEDATRQMREFVRTIKRPFDVHYNPYTQSIEIIKTPKSVAKLVQDLQFELTAINESLLKMNKEIRSQQFTTNKIVTENRSSGS

SEQ ID NO: 48 - protein, 345 aa, Homo sapiens
MGSSEDQAYRLLNDYANGFMVSQVLFAACELGVFDLLAEAPGPLDVAAVAAGVRASAHGTELLLDICVSLKLLKVETRGGKAFYRNTELSSDYLTTVSPTSQCSMLKYMGRTSYRCWGHLADAVREGRNQYLETFGVPAEELFTAIYRSEGERLQFMQALQEVWSVNGRSVLTAFDLSVFPLMCDLGGGAGALAKECMSLYPGCKITVFDIPEVVWTAKQHFSFQEEEQIDFQEGDFFKDPLPEADLYILARVLHDWADGKCSHLLERIYHTCKPGGGILVIESLLDEDRRGPLLTQLYSLNMLVQTEGQERTPTHYHMLLSSAGFRDFQFKKTGAIYDAILARK

SEQ ID NO: 49 - protein, 356 aa, Tabernanthe iboga
DAMKSAELFKAQAHIFKQVFCFTNGASLKCAVQLGIPDAIDNHGKAMTLSELTDALPINPSKAPHIHRLMRILVTAGFFVEERLGNGKEEKANGYALTPSSRLLLKNKPLSLRASALTMLDPVTVKTWNALSEWFQNEDQTAFETAHGKNMWDFFAEDPGLSKKFNESMASDSQLVTEVLVTKCKFVFEGLTSMVDVGGGTGTVAGAIAKTFPSLRCTVFDLPHVVANLEPTENLDFVAGDMFGKIPPANAIFLKWVLHDWNDEDCVKILKNCKRAIPGKEKGGKVIIVDIIMETEKHDIDEFDYAKMCMDMEMLVLCNSKERTEKELAMLVSEAGFSGYKIFPVLGIRSLIEVYP